(19) United States
(12) Patent Application Publication
(10) Pub. No.: US 2004/0172670 A1
(43) Pub. Date: Sep. 2, 2004

(54) Maize yellow stripe1 and related genes

(76) Inventors: Elsbeth L. Walker, Amherst, MA (US); Stephen Dellaporta, Branford, CT (US)

Correspondence Address: MORGAN LEWIS & BOCKIUS LLP, 1111 PENNSYLVANIA AVENUE NW, WASHINGTON, DC, 20004, US

(21) Appl. No.: 10/416,898

(22) PCT Filed: Nov. 16, 2001
(86) PCT No.: PCT/US01/43101

Related U.S. Application Data
(60) Provisional application No. 60/249,222, filed on Nov. 16, 2000.

(51) Int. Cl. A01H 1/00; C12N 15/82; C07H 21/04; A01H 5/00; C12N 5/04

(52) U.S. Cl. 800/278; 800/320.1; 435/69.1; 435/320.1; 435/412; 530/370; 536/23.6

(57) ABSTRACT

The maize yellow stripe1 gene encodes a transmembrane protein which mediates the uptake of chelated iron from soil. The gene is shown here to be upregulated, and protein production increased, under conditions of low iron bioavailability. The protein is also shown to be able to transport other metals as well, such as copper. The present invention also provides a family of Arabidopsis genes and gene products related to ys1 termed here as ys11-8. The invention provides the genes and their gene products as well as methods to create and use transgenic plants for the increased uptake of iron in nutritional crops as well as for the bioremediation of soil.

### CROSS REFERENCES TO RELATED APPLICATIONS

This application claims priority to Provisional Application 60/249,222, filed Nov. 16, 2000, which is hereby specifically incorporated by reference in its entirety.

### STATEMENT OF RIGHTS TO INVENTIONS MADE UNDER FEDERALLY SPONSORED RESEARCH

This invention was partially made with government support under United States National Institutes of Health Grant No. R01 GM38148 and under United States Department of Agriculture NRICGP Grant No. 99-35100-7601.

### FIELD OF THE INVENTION

This invention relates generally to maize proteins responsible for the uptake of iron and other metals, such as heavy metals, from soil, genes encoding said proteins, vectors comprising said genes, recombinant prokaryotic and eukaryotic cells comprising said genes and the use of said vectors to create transgenic plant cells, plant tissues and whole plants. More specifically, this invention relates to the cloning and isolation of the maize yellow stripe 1 (ys1) gene and the yellow stripe1-like (ysl) genes of Arabidopsis. In addition, this invention also provides methods of using ys1 or ysl transgenic plants for enhancing iron uptake from soil and for bioremediation of metal or heavy metal contaminated soil. Further, this invention also provides the engineering of ys1 or ysl transgenic plants in order to alter the distribution of Fe within the plant body, e.g., so that edible parts of crop plants have more iron.

### BACKGROUND OF THE INVENTION

All publications and patent applications herein are incorporated by reference to the same extent as if each individual publication or patent application was specifically and individually indicated to be incorporated by reference. Portions of this disclosure have been reported in Curie, C. et al. Nature 2001 49:346-349, which is specifically incorporated by reference in its entirety.

Iron deficiency is the most prevalent human nutritional problem in the world today, affecting an estimated 3 billion people in both industrial and developing countries according to statistics from the National Science Foundation (USA) and the World Health Organization. Frequently, crop plants do not take up adequate amounts of iron from the soil, leading to chlorosis, poor yield and decreased nutritional quality. Plants serve as the principal source of iron in most diets worldwide. Unfortunately, most crops contain low amounts of bioavailable iron. Additionally, low iron availability in soil often limits plant growth, resulting in reductions in crop yield. Approximately one-third of the world's soils are iron deficient. Deficiency of iron is not the only factor that reduces iron uptake by plants. Plant uptake of iron may also be limited due to conditions such as: high soil pH (alkalinity), high lime content in soil, calcareous soil, excess phosphates in the soil, irrigation water containing high levels of bicarbonate ions, excess moisture along with low soil temperatures and excess amounts of copper and manganese in acidic soils. Iron bioavailability in soil is particularly affected by high pH, becoming oxidized to become Fe₂O₃. Additionally, iron deficiency in soil can occur with heavy application of high phosphorous fertilizers, high Cu concentration in the soil and abnormally high or low levels of manganese (Hausenbuiller, R L. in Soil Science: 2nd Edition. (1978) Wm. C. Brown Co. Dubuque, Iowa, pages 339-362).

Iron deficiency in plants causes chlorosis, visually characterized by yellowing of the tissue between the veins of leaves while the veins themselves stay green. As it advances through the plant, the tips and margins of leaves may start to turn brown and become dry and brittle. Severe cases may result in necrotic spots on the chlorotic leaves or in the death of the plant. Limited bioavailability of iron has led to the evolution of uptake strategies that can be broadly defined as chelation, i.e., specific extrusion and re-uptake of molecules that bind iron; and reduction, i.e., plasma membrane localized ferric reductases coupled with iron transporters (Briat, J-F et al. Trends Plant Sci. 1997, 2:187-193; Mori, S. Curr. Opin. Plant Biol. 1999, 2:250-253; Yi, Y et al. Plant J. 1996 10:835-844).

Under conditions of iron insufficiency dicotyledonous plants and non-graminaceous monocots, collectively known as Strategy I plants 03riat, J-F et al. Trends Plant Sci. 1997 2:187-193), adopt a reduction strategy. These organisms solubilize ferric iron by acidification of their environment due to proton extrusion, and then enzymatically reduce insoluble iron (Fe[III]) in the soil surrounding the roots via membrane-bound reductases, enabling the subsequent uptake of ferrous iron by Fe[II] transporters. A root ferric-chelate reductase (FRO2) that is up-regulated upon iron starvation, and a root Fe[II] transporter (IRT1) from Arabidopsis thaliana have recently been cloned and characterized (Yi, Y et al. Plant J. 1996 10:835-844; Robinson, N.J. et al. Nature 1999 397:694-697).

Graminaceous plant species acquire iron by a strategy (called Strategy II) involving ferric iron chelation by low-molecular weight secondary amino acids of the mugineic acid (MA) fanily called phytosiderophores (Briat, J-F et al. Trends Plant Sci. 1997 2:187-193). These compounds function as hexadentate cation chelators (Tagaki, S et al. J. Plant Nutr. 1984 7:469-477). As a response to iron-deficiency stress, phytosiderophores are synthesized from methionine precursors via nicotinamine. When released from plant roots, phytosiderophores can chelate sparingly soluble iron, as from Fe hydroxides or phosphates. Iron acquisition via this strategy is probably very advantageous in soils with high pH and/or high levels of bicarbonate where release of protons is ineffective in solubilizing iron, and ferric reductase activity is inhibited. This explains the ecological advantage of grasses, compared to non-graminaceous plant species under conditions where iron is either deficient in the soil or otherwise of limited bioavailability. phytosiderophore-mediated uptake of iron is further reviewed by S. Mori (The role of mugineic acid in iron acquisition: progress in cloning the genes for transgenic rice. In: Plant Nutrient Acquisition. Ae, N., Arihara, N., Okada, K, and A. Srinivasan, eds. 2001. Springer-Verlag, Tokyo, incorporated herein in its entirety).

Studies indicate that ferric reduction is not a prerequisite for phytosiderophore-mediated uptake. Physiological conditions which block ferric reductase activity (e.g., high pH) do not block uptake of Fe[III]. MA, and the strong Fe[II] chelator 4,7-biphenyl-1,10-phenanthroline-disulphonic acid (BPDS), which is very effective at blocking iron uptake in Strategy I plants, is ineffective in blocking Fe[III]. MA uptake. By analogy to bacterial siderophore-mediated iron uptake, Strategy II plants are thought to transport the entire Fe.phytosiderophore complex. Evidence for this idea comes from double labeling studies using ⁵⁹Fe[III]. [¹⁴C] deoxymugineic acid (DMA) (von Wiren, N et al. Plant Physiol. 1995 106:71-77).

Two iron uptake mutants, yellowstripe1 (ys1) and yellowstripe3 (ys3), have been reported in maize, a graminaceous Strategy II plant. Both mutants have similar phenotypes of chlorotic yellow striped leaves, which can be reversed by application of iron directly to the leaves. The maize ys1 mutant has been used in many physiological studies addressing the mechanism of phytosiderophore uptake. Several studies have indicated that YS1 is involved in phytosiderophore-mediated iron uptake in maize. While wild-type (WT) maize appears to be capable of using barley phytosiderophores in co-culture, homozygous ys1 mutants were not, based on their chlorotic appearance (Jolley, V D et al. J. Plant Nutr. 1991 14:45-58; Hopkins, B G et al. J. Plant Nutr. 1992 15:1599-1612). It was later confirmed that ys1 mutant plants produce normal amounts of the maize phytosiderophore, DMA. Both uptake and translocation to shoots of ⁵⁹Fe from ⁵⁹Fe-DMA was more than 20-fold lower for ys1 mutant plants than for WT controls. These uptake experiments suggested that YS1 is a high affinity Fe[III] transporter with a Kₘ in the range of 10 μM (von Wiren, N et al. Plant Physiol. 1995 106:71-77).

The present inventors have for the first time cloned the maize ys1 gene and isolated the YS1 protein. YS1 is shown here to be a novel protein that shares structural features of integral membrane proteins. It restores growth of a yeast mutant defective in iron uptake specifically on an Fe-DMA containing medium. Furthermore, the ysl gene is shown here to be up-regulated in response to iron starvation both in roots and shoots.

Thus, an object of this invention is to satisfy a long felt need in the art for improving the ability of food plants to uptake nutritionally significant amounts of iron from soils in which the bioavailability of iron is limited due to deficiency in the soil or other conditions which inhibit iron uptake by plants. The present invention provides for the making of transgenic plants that express the ysl gene of the present invention under conditions of low iron bioavailability.

While plants require iron for normal growth and development, and sufficient levels of iron in plant matter are desirable for nutritional value, iron can also be toxic to plants if accumulated to high levels. Accordingly, in soils where iron is overabundant, crop yield is also reduced. The present inventors show here that, under control of its native promoter, expression of ys1 is down-regulated in conditions of iron over-availability.

Thus, a further object of this invention is the creation of vectors wherein the expression of ys1 is not down-regulated by high iron levels in order to provide transgenic plants that are tolerant of high iron levels in soil and can accumulate higher iron levels from the soil. These transgenic plants are useful either for their own nutritional value or in order to condition soil for the growth of plants that are not tolerant of, e.g., reduced in their ability to thrive in, soils which are overly iron-rich. For example, ys1 transgenic plants can be co-cultivated with said plants that are not tolerant of soils which are overly iron-rich in order to temporarily reduce local iron concentrations around the less tolerant plants. Accordingly, the method would allow the temporary local depletion of iron in an area of soil without long-tern reduction of bioavailable iron for future crops.

Up to 12 percent of soils under cultivation around the world contain high concentrations of metals, including heavy metals such as copper and magnesium, which stunt plant growth and development and result in poor harvests. In addition, metal contamination of soil in general, such as from industrial waste, poses significant threats to health worldwide. Of particular interest in regard to metal contamination in soils is contamination by heavy metals, many of which are toxic to plants and/or animals. The use of plants to remove metals from the soil is known as phytoremediation and is often also referred to as phytoextraction, bioremediation, botanical-bioremediation, and Green Remediation. Phytoextraction is further reviewed, for example, by S. P. McGrath in Plants that Hyperaccumulate Heavy Metals: Their Role in Phytoremediation, Microbiology, Archaeology, Mineral Exploration and Phytomining. (1998) CAB International. Oxon, UK, pages 261-287. The present inventors have surprisingly found, and disclose in this application, that ys1 is capable of the uptake of other metals besides iron, such as copper. However, maize is a large, high biomass, plant that, while capable of high levels of uptake, is not capable of high levels of storage, or accumulation, of metals. Therefore, use of maize for bioremediation would not be cost effective and may cause new waste disposal issues. Plants having a metal hyperaccumulator phenotype is much more important than high plant-matter yield ability when using plants to remove metals from contaminated soils. One such hyperaccumulator of metals is Thlaspi caerulescens which can, for example, hypertolerate up to about 25,000 mg Zn per Kg of plant biomass, compared to a significant crop yield reduction at 500 mg Zn per Kg plant biomass for Zea mays. Other exemplary hyperaccumulator plants would include, but are not limited to Amaranthus paniculata, Brassicajuncea, B. carinata, B. oleracea, B. nigra, B. campestris, B. napus, B. tournifortii, Raphanus sativus, Sinapis alba, S. arvensis, S. flexuosa and S. pubescens. The process of hyperaccumulation and further exemplary hyperaccumulator plants are described, for example, by R. R. Brooks (in “Plants that Hyperaccumulate Heavy Metals: Their Role in Phytoremediation, Microbiology, Archaeology, Mineral Exploration and Phytomining.” (1998) CAB International. Oxon, UK, pages 55-94), by Reeves R D et al. (in “Phytoremediation of Toxic Metals.” (2000) John Wiley & Sons, Inc. New York. pages 193-229) and Salt D E et al. (in “Phytoremediation of Toxic Metals.” (2000) John Wiley & Sons, Inc. New York. pages 25 231-246).

Plants which are hyperaccumulators must be able to tolerate high levels of the metal in root and shoot cells (hypertolerance), with vacuolar compartmentalization of metals appearing to be the source of hypertojerance of many natural hyperaccumulator plants. A plant must have the ability to translocate an element from roots to shoots at high rates. Normally root metal concentrations are 10 or more times higher than shoot con centrations, but in hyperaccumulators, shoot metal concentrations can exceed root levels (Chaney, R L et al. Curr. Opin. Biotechnol. 1997, 8:279-284; Vogeli-Lange R, et al. Plant Physiol 1990, 92:1086-1093; Ortiz D F, et al. J. Biol Chem 1995, 270:47214728; Guerinot, M L in “Thytoremediation of Toxic Metals.” (2000) John Wiley & Sons, Inc. New York. pages 193-229). While hyperaccumulators are capable of tolerating high concentrations of metals, they are frequently slow growing and may not uptake metal from the soil any faster than plants which the metals would be toxic to. For example, while T. caerulescens accumulated Zn from nutrient solution only about as well as tomato, tomato was severely injured at 30 μM Zn while T. caerulescens was not severely injured until 10,000 μM Zn (Brown S L, et al. Soil Sci Soc Am J 1995, 59:125-133).

An alternative method to hyperaccumulation in the handling of metals by plants in phytoremediation is known as volatilization. Volatilization is described, for example, by R. R. Brooks in Plants that Hyperaccumulate Heavy Metals: Their Role in Phytoremediation, Microbiology, Archaeology, Mineral Exploration and Phytomining. (1998) CAB International. Oxon, UK, pages 289-312.

Little molecular understanding of plant activities critical to phytoremediation has been achieved, but recent progress in characterizing Fe, Cd, and Zn uptake by Arabidopsis and yeast mutants indicates strategies for developing transgenic-improved phytoremediation cultivars for commercial use. In addition, the present inventors have found a group of related yellow stripe1-like (YSL) proteins present in Arabidopsis which are also capable of metal uptake.

### SUMMARY OF THE INVENTION

The instant invention is directed to the maize yellow stripe1 (ys1) gene (SEQ ID NO: 1) and the protein product of the gene (SEQ ID NO: 2). The sequence of the ysl cDNA has been deposited under the GenBank Accession Number AF186234. The instant invention is further directed to the yellow stripe1-like (ysl) genes (SEQ ID NOs: 3, 5, 7, 9, 11, 13, 15 and 17) of Arabidopsis and the protein products of those genes (SEQ ID NOs: 4, 6, 8, 10, 12, 14, 16 and 18, respectively). The inventors have discovered that the ysl gene product is responsible for phytosiderophore-mediated iron uptake in maize. The inventors have found and disclose here that YS1 can be transferred into other organisms and mediate phytosiderophore-mediated iron uptake in those organisms. The present inventors have also surprisingly discovered that YS1 can also mediate the uptake of other metals into transformed organisms. The disclosed nucleic acid molecules of the present invention encode proteins which act as metal ion transporters and the invention thus allows one to alter metal ion homeostasis in any plant by altering the pattern and/or level of expression of the disclosed nucleic acid molecules. Thus, the nucleic acids of the present invention can be used to confer unique and agronomically useful traits upon any plant desired, wherein such traits are highly desirable and commercially valuable.

One object of the present invention is to provide maize ysl nucleic acids and the YS1 protein produced thereby. The present invention also provides ysl nucleic acids of Arabidopsis and the YSL proteins they produce. The invention includes isolated nucleic acid molecules selected from the group consisting of isolated nucleic acid molecules that encode an amino acid sequence selected from the group consisting of SEQ ID NO: 2, 4, 6, 8, 10, 12, 14, 16 and 18, an isolated nucleic acid molecule that encodes a fragment of at least 6 amino acids of SEQ ID NO: 2, 4, 6, 8, 10, 12, 14, 16 or 18 and an isolated nucleic acid molecule which hybridizes to a nucleic acid molecule comprising SEQ ID NO: 1, 3, 5, 7, 9, 11, 13, 15 or 17. A nucleic acid molecule can include functional equivalents of natural nucleic acid molecules encoding a protein of the present invention. Functional equivalents of natural nucleic acid molecules can include, but are not limited to, natural allelic variants and modified nucleic acid molecules in which nucleotides have been inserted, deleted, substituted, and/or inverted in such a manner that such modifications do not substantially interfere with the nucleic acid molecule's ability to encode a molecule of the present invention. Said amino acid substitutions may be conservative or non-conservative. Preferred functional equivalents include sequences capable of hybridizing under stringent conditions (ie. sequences having at least about 70% identity), to at least a portion of a signal transduction protein encoding nucleic acid molecule according to conditions described in Sambrook et al., (1989) Molecular Cloning—A Laboratory Manual, Cold Spring Harbor Laboratory Press. By stringent conditions it is meant that hybridization is carried in a buffer consisting of 0.1% SDS , 200 mM NaCl, 6 mM Na₂HPO₄, 2 mM EDTA at pH=6.8. More preferred functional equivalents include sequences capable of hybridizing under stringent conditions (i.e., sequences having at least about 90% identity), to at least a portion of a signal transduction protein encoding nucleic acid molecule. By highly stringent conditions it is meant that hybridization is carried in a buffer consisting of 0.1% SDS, 10 mM NaCl, 0.3 mM Na₂HPO₄, 0.1 mM EDTA at pH=6.8. Nucleic acid molecules of the invention may encode a protein having at least about 50 or 60% amino acid sequence identity with the sequence set forth in SEQ ID NO: 2, preferably at least about 70 or 75%, more preferably at least about 80%, still more preferably at least about 85%, yet more preferably at least about 90%, even more preferably at least about 95% and most preferably at least about 98% sequence identity with the protein sequence set forth in SEQ ID NO: 2, 4, 6, 8, 10, 12, 14, 16 or 18.

The present invention further includes the nucleic acid molecules operably linked to one or more expression control elements, including vectors comprising the isolated nucleic acid molecules. The invention further includes host cells transformed to contain the nucleic acid molecules of the invention and methods for producing a protein comprising the step of culturing a host cell transformed with a nucleic acid molecule of the invention under conditions in which the protein is expressed.

The invention further provides an isolated polypeptide selected from the group consisting of an isolated polypeptide comprising the amino acid sequence of SEQ ID NO: 2, 4, 6, 8, 10, 12, 14, 16 or 18, an isolated polypeptide comprising a fragment of at least 6 amino acids of SEQ ID NO: 2, 4, 6, 8, 10, 12, 14, 16 or 18, an isolated polypeptide comprising conservative amino acid substitutions of SEQ ID NO: 2, 4, 6, 8, 10, 12, 14, 16 or 18 and an isolated polypeptide comprising naturally occurring amino acid sequence variants of SEQ ID NO: 2,4, 6, 8, 10, 12, 14, 16 or 18. Polypeptides of the invention also include polypeptides with an amino acid sequence having at least about 50 or 60% amino acid sequence identity with the sequence set forth in SEQ ID NO: 2, preferably at least about 70 or 75%, more preferably at least about 80%, still more preferably at least about 5%, yet more preferably at least about 90%, even more preferably at least about 95% and most preferably at least about 98% sequence identity with the protein sequence set forth in SEQ ID NO: 2, 4, 6, 8, 10, 12, 14, 16 or 18.

This invention provides vectors comprising the nucleic acid constructs of the present invention as well as host cells, recombinant plant cells and transgenic plants comprising the vectors of the present invention. More particularly, this invention provides such cells and transgenic plants that are hemizygotic, heterozygotic or homozygotic for the nucleic acid constructs, wherein such plants can be monoploid, diploid or polyploid. It is an object of the present invention to provide such cells and transgenic plants wherein they express a single copy or multiple copies of one or more of the YS1 or YSL protein products of the present invention. Cells or transgenic plants which express multiple copies of one of the YS1 or YSL proteins, or which express more than one of the YS1 or YSL proteins, may be desirable, for example, to enhance the uptake of metals into the cell or transgenic plant or to broaden the range or types of metals taken up by the cell or transgenic plant.

The invention further provides nucleic acid probes for the detection of expression of YS1 and/or YSL, or homologues or orthologues thereof, in plants which either have been genetically altered to express at least one of said proteins or which may naturally express YS1, a YSL protein or homologues or orthologues thereof. The invention further provides the use of antibodies to YS1, a YSL protein or to a homologue or orthologue thereof to probe a biological sample or a tissue section for expression of YS1, a YSL protein or a homologue or orthologue thereof. Said biological sample or tissue section may be from a plant which has been genetically altered to express said protein or which may naturally express YS1, a YSL protein or a homologue or orthologue thereof.

A further object of this invention is to satisfy a long felt need in the art for improving the ability of plants to uptake nutritionally significant amounts of a metal, such as iron, from soils and to alter the deposition of the metal in the plants so as to obtain increased metal micronutrient content in the edible or otherwise useable plant parts. Thus, the present invention provides for the production of transgenic plants that express at least one of the ys1 or ysl gene products of the present invention so as to alter the pattern of deposition of metal ions in a plant under any particular growing conditions. The transgenic plants of the present invention can be grown in any suitable medium, including but not limited to soil, sand, Perlite, Vermiculite, hydroponics, etc. In addition, the transgenic plants of the present invention can be used to accumulate specific metals in specific plant parts under conditions of low, average or high concentrations of the targeted metals.

A further object of this invention is to satisfy a long felt need in the art for improving the ability of food plants to uptake nutritionally significant amounts of iron from soils in which the bioavailability of iron is limited due to deficiency in the soil or other conditions which inhibit iron uptake by plants. Thus, the present invention provides for the production of transgenic plants which express at least one of the ys1 or ysl gene products of the present invention under conditions of low iron bioavailability.

A further object of this invention is the creation of vectors wherein the expression of ys1 or ysl gene is not down-regulated by normal or high iron levels so as to provide transgenic plants which are tolerant of high iron levels in soil and can accumulate higher iron levels from the soil. For example, such a vector would replace the iron-regulated promoter normally associated with ys1 with a promoter that permits continuous expression of ys1. These transgenic plants are useful either for their own nutritional value or in order to prepare soil for the growth of plants that are not tolerant of—or are reduced in their ability to thrive in—soils that are overly iron-rich. Accordingly, the invention provides for vectors comprising ys1 or ysl coding sequence under the control of a primer that is not down-regulated in conditions of high iron or other heavy metal concentrations. Said promoter may be located on the same vector or on a separate vector.

Another object of this invention is to provide a transgenic plant that expresses at least one of the YS1 or YSL proteins in order to facilitate, accelerate, enhance and/or increase uptake of heavy metal from the soil. Transgenic plants may be natural hyperaccumulators of heavy metals or may be additionally engineered to express a hyperaccumulator phenotype. The disclosed nucleic acids can also be used to alter the pattern of deposition of metal ions, allowing for more efficient transport of the metals to tissues capable of sequestering high levels of metal ions.

The invention further provides methods for using such transgenic plants in bioremediation.

Other objects, advantages and features of the present invention become apparent to one skilled in the art upon reviewing the specification and the drawings provided herein.

### BRIEF DESCRIPTION OF THE DRAWINGS

FIG. 1A. Map of the 9.5 kb SalI fragment contained in the λYS3 1 genomic clone. The positions of the Ac element and the probe fragment YS1-F are indicated.

FIG. 1B. Map of the ys1 gene. Exons are indicated by black boxes. The positions of the Ac element in the ys1-m1::Ac allele and the retrotransposon element in the ys1:ref allele are indicated above and below. The probe fragment YS1-F is also shown.

### DETAILED DESCRIPTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

It will be appreciated from the above that the tools and methods of the present invention have application to all plants that produce gametes. Such plants include, but are not limited to, forage grasses, turf grasses, ornamental grasses, forage legumes, ground covers, vegetables, field crops (e.g., soybeans, corn, rice, cotton, tobacco, sorghum, field peas), trees and ornamental flowers.

Definitions

As used herein, the term “allele” refers to any of several alternative forms of a gene.

As used herein, the term “chelating agent” refers to any chemical compound which attaches to a metal ion such that the metal ion is attached to at least two nonmetal chemical compounds in order to form a heterocyclic ring. Many chelating agents will form soluble or partially soluble complexes with metal ions which can make the metal more available to the plants and allow the plants to accumulate a particular metal. Other chelating agents may form insoluble complexes with metals and serve to: (i) concentrate metals so they may be physically or chemically accumulated (i.e., sorbed) onto roots of the plants; and/ or (ii) prevent leaching or other removal of metals from the vicinity of the root zone. Examples of chelating agents include, but are not limited to, the following: ammonium purpurate (murexide), 2,3-butane-dione dioxime (dimethylglyoxime), 3,6 disulfo-1,8-dihydroxynaphthalene (chromotroic acid), and thiourea, alpha-benzoin oxime (cupron), trans-1,2-diaminocyclohexanetetraacetic acid (CDTA), diethylene-triaminopentaacetic acid (DTPA), 2,3-dimercapto-1-propanol, diphenylthiocarbazone, nitrilotriacetic acid (NTA), substituted 1,10-phenanthrolines (e.g., 5-nitro-1,10 phenanthroline), sodium deithyldithiocarbamate (cupral), 2-thenoyl-2-furoylmethane, thenoyl-trifluoroacetone, triethylenetetramine, and ethylenediaminetetraacetic acid (EDTA) and citric acid. See, for example, the reference Dawson et al., (eds), “Stability Constants of Metal Complexes”, pp. 399-415 in Data for Biochemical Research, Claredon Press, Oxford, UK, 1986, which is incorporated herein in its entirety by reference.

As used herein, the term “crop plant” refers to any plant grown for any commercial purpose, including, but not limited to the following purposes: seed production, hay production, ornamental use, fruit production, berry production, vegetable production, oil production, protein production, forage production, animal grazing, golf courses, lawns, flower production, landscaping, erosion control, green manure, improving soil tilth/health, producing pharmaceutical products/drugs, producing food or food additives, smoking products, pulp production and wood production.

As used herein, the term “cross pollination” or “cross-breeding” refer to the process by which the pollen of one flower on one plant is applied (artificially or naturally) to the ovule (stigma) of a flower on another plant.

As used herein, the term “cultivar” refers to a variety, strain or race of plant that has been produced by horticultural or agronomic techniques and is not normally found in wild populations.

As used herein, the term “female” refers to a plant that produces ovules. Female plants generally produce seeds after fertilization. A plant designated as a “female plant” may contain both male and female sexual organs. Alternatively, the “female plant” may only contain female sexual organs either naturally (e.g., in dioecious species) or due to emasculation (e.g., by detasselling).

As used herein, the term “filial generation” refers to any of the generations of cells, tissues or organisms following a particular parental generation. The generation resulting from a mating of the parents is the first filial generation (designated as “F1” or “F₁”), while that resulting from crossing of F1 individuals is the second filial generation (designated as “F2” or “F₂”).

As used herein, the term “gamete” refers to a reproductive cell whose nucleus (and often cytoplasm) fuses with that of another gamete of similar origin but of opposite sex to form a zygote, which has the potential to develop into a new individual. Gametes are haploid and are differentiated into male and female.

As used herein, the term “gene” refers to any segment of DNA associated with a biological function. Thus, genes include, but are not limited to, coding sequences and/or the regulatory sequences required for their expression. Genes can also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “genotype” refers to the genetic makeup of an individual cell, cell culture, tissue, plant, or group of plants.

As used herein, the terms “heterologous polynucleotide” or a “heterologous nucleic acid” or an “exogenous DNA segment” refer to a polynucleotide, nucleic acid or DNA segment that originates from a source foreign to the particular host cell, or, if from the same source, is modified from its original form. Thus, a heterologous gene in a host cell includes a gene that is endogenous to the particular host cell, but has been modified. Thus, the terms refer to a DNA segment which is foreign or heterologous to the cell, or homologous to the cell but in a position within the host cell nucleic acid in which the element is not ordinarily found. Exogenous DNA segments are expressed to yield exogenous polypeptides.

As used herein, the term “heterologous trait” refers to a phenotype imparted to a transformed host cell or transgenic organism by an exogenous DNA segment, heterologous polynucleotide or heterologous nucleic acid.

As used herein, the term “heterozygote” refers to a diploid or polyploid individual cell or plant having different alleles (forms of a given gene) present at least at one locus.

As used herein, the term “heterozygqus” refers to the presence of different alleles (forms of a given gene) at a particular gene locus.

As used herein, the term “homologue” refers to a nucleic acid or peptide sequence which has a common origin and functions similarly to a nucleic acid or peptide sequence from another species.

As used herein, the term “homozygote” refers to an individual cell or plant having the same alleles at one or more loci.

As used herein, the term “homozygous” refers to the presence of identical alleles at one or more loci in homologous chromosomal segments.

As used herein, the term “hybrid” refers to any individual cell, tissue or plant resulting from a cross between parents that differ in one or more genes.

As used herein, the term “hyperaccumulator” refers to any plant that is able to uptake and store within its tissues an amount of heavy metal that is a greater percentage of its dry biomass when compared to its wild-type counterpart. More particularly, a hyperaccumulator is a plant that is capable of storing an amount of a heavy metal that is at least, equal to or greater than about 0.5% of said plant's dry biomass. Preferably, a hyperaccumulator is a plant that is capable of storing an amount of a heavy metal that is at least, equal to or greater than about 1.0% of said plant's dry biomass. More preferably, a hyperaccumulator is a plant that is capable of storing an amount of a heavy metal that is at least, equal to or greater than about 1.5% of said plant's dry biomass. Even more preferably, a hyperaccumulator is a plant that is capable of storing an amount of a heavy metal that is at least, equal to or greater than about 2.0% of said plant's dry biomass. Most preferably, a hyperaccumulator is a plant that is capable of storing an amount of a heavy metal that is at least, equal to or greater than about 2.5% of said plant's dry biomass. Optimally, a hyperaccumulator is a plant that is capable of storing an amount of a heavy metal that is at least, equal to or greater than about 5.0% of said plant's dry biomass. Alternatively, a hyperaccumulator can be defined as any plant that can uptake and accumulate at least about 10 times more metal in shoots on a dry weight basis that the amount of metal present in the metal-containing soil, or which are able to accumulate at least about 20 times more metal in roots on a dry weight basis that the amount of metal present in the metal-containing soil.

Examples of hyperaccumulator plants include, but are not limited to, the following: Alyssum pinifolium, Amaranthus paniculata, Bornmuellera baldaccii ssp. markgrasii, Brassica juncea, B. carinata, B. oleracea, B. nigra, B. campestris, B. napus, B. nigra, B. tournifortii, Raphanus sativus (L.)(radish), Calodophora species, Dichapetalum gelonioides, Rumex scutatus, Sinapis alba (L.)(white mustard), S. arvensis (L.), S. flexuosa and S. pubescens (L.), Thlaspi alpestre var. calaminare, Trifolium arvense, Thlaspi rotundifolium, Thlaspi caerulescens, Thlaspi goesingense, Viola calaminaria, Zea mays, Agrostis capillaries, and Larrea tridentate, (U.S. Pat. Nos. 5,927,005; 6,159,270; Huang, J W et al. New Phytol. 1996 134:75-84; Cotter-Howells, J D et al. Appi. Geochem. 1996 11:335-342; Vazquez, M D et al. J&C Presi. Bot. Acta 1994 107:243-250; Reeves, R. D. et al. In Phytoremediation of Toxic Metals, I. Raskin & B. D. Ensley, eds., John Wiley & Sons, Inc.2000 Ch. 12:193-229).

As used herein, the term “hyperaccumulator gene” refers to any nucleic acid sequence which encodes for a gene product which confers upon a wild-type, genetically engineered or manipulated plant a hyperaccumulator phenotype.

As used herein, the term “inbred” or “inbred line” refers to a relatively true-breeding strain.

As used herein, the term “knock-in” refers to a cell, tissue or organism that has had a gene introduced into its genome, wherein the gene can be of exogenous or endogenous origin. Generally, if the introduced gene is endogenous in origin, it will be a modified gene. An introduced gene that is exogenous in origin can be in its wild-type form or in a modified form.

As used herein, a “knock-out” refers to a cell, tissue or organism in which there is partial or complete suppression of the expression of an endogenous gene (e.g., based on deletion of at least a portion of the gene, replacement of at least a portion of the gene with a second sequence, introduction of stop codons, the mutation of bases encoding critical amino acids, or the removal of an intron junction, etc.). The targeted gene can be partially or completely suppressed by disruption, inactivation or deletion. Said partial suppression may also be referred to herein as a “knock-down.” Knock-outs can be performed using both in vitro and in vivo recombination techniques. In order to study gene functions, usually the cell, tissue or organism is genetically engineered with specified wild-type alleles replaced with mutated ones. Knock-outs can be made using homologous recombination between the target gene and a piece of cloned DNA to insert a piece of “junk” DNA into the gene desired to be disrupted. If the organism is haploid, then this technique will result in that organism's only copy of the gene being knocked out. If it is diploid, then only one of the two alleles will be knocked out, and it will be necessary to do conventional breeding to produce a diploid organism that has two copies of the gene knocked out.

As used herein, the term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (T0) plant regenerated from material of that line; (b) has a pedigree comprised of a T0 plant of that line; or (c) is genetically very similar due to common ancestry (e.g., via inbreeding or selfing). In this context, the term “pedigree” denotes the lineage of a plant, e.g. in terms of the sexual crosses effected such that a gene or a combination of genes, in heterozygous (hemizygous) or homozygous condition, imparts a desired trait to the plant.

As used herein, the term “locus” (plural: “loci”) refers to any site that has been defined genetically. A locus may be a gene, or part of a gene, or a DNA sequence that has some regulatory role, and may be occupied by different sequences.

As used herein, the term “male” refers to a plant that produces pollen grains. The “male plant” generally refers to the sex that produces gametes for fertilizing ova. A plant designated as a “male plant” may contain both male and female sexual organs. Alternatively, the “male plant” may only contain male sexual organs either naturally (e.g., in dioecious species) or due to emasculation (e.g., by removing the ovary).

As used herein, the term “mass selection” refers to a form of selection in which individual plants are selected and the next generation propagated from the aggregate of their seeds.

As used herein, the term “metal” preferably refers to metal ions that are found in the metal containing environment. It will be appreciated that this term will also include elemental metal that is not in an ionic form. The metals that can be accumulated according to the method of the present invention include stable metals and radioactive metals such as lead, chromium, mercury, cadmium, cobalt, barium, nickel, molybdenum, copper, arsenic, selenium, zinc, antimony, beryllium, gold, manganese, silver, thallium, tin, rubidium, vanadium, strontium, yttrium, technecium, ruthenium, palladium, indium, cesium, uranium, plutonium, and cerium. The term “metal” is also intended to include more than one metal since plants may concentrate several different metals, implying that the mechanism of metal uptake is not always metal specific. The term “metal” also includes mixtures of metals and common organic pollutants such as, for example, lead or chromium in combination with nitrophenol, benzene, alkyl benzyl sulfonates (detergents), polychlorinated biphenyls (PCB's) and/or halogenated hydrocarbons (e.g., trichloroethylene). The term “metal” also encompasses and may preferably be a “heavy metal,” which includes any metal with a specific gravity of at least about 5.0. The term “metal” further encompasses any metal which may be of nutritional value to one who consumes the plant. The term “metal” further encompasses any metal which is poisonous to an organism which consumes or comes in contact with it.

As used herein, the terms “nucleic acid” or “polynucleotide” refer to deoxyribonucleotides or ribonucleotides and polymers thereof in either single- or double-stranded form. Unless specifically limited, the terms encompass nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) 3. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene. The term “nucleic acid” also encompasses polynucleotides synthesized in a laboratory using procedures well known to those skilled in the art.

As used herein, a DNA segment is referred to as “operably linked” when it is placed into a functional relationship with another DNA segment. For example, DNA for a signal sequence is operably linked to DNA encoding a polypeptide if it is expressed as a preprotein that participates in the secretion of the polypeptide; a promoter or enhancer is operably linked to a coding sequence if it stimulates the transcription of the sequence. Generally, DNA sequences that are operably linked are contiguous, and in the case of a signal sequence both contiguous and in reading phase. However, enhancers need not be contiguous with the coding sequences whose transcription they control. Linking is accomplished by ligation at convenient restriction sites or at adapters or linkers inserted in lieu thereof.

As used herein, the term “open pollination” refers to a plant population that is freely exposed to some gene flow, as opposed to a closed one in which there is an effective barrier to gene flow.

As used herein, the terms “open-pollinated population” or “open-pollinated variety” refer to plants normally capable of at least some cross-fertilization, selected to a standard, that may show variation but that also have one or more genotypic or phenotypic characteristics by which the population or the variety can be differentiated from others. A hybrid, which has no barriers to cross-pollination, is an open-pollinated population or an open-pollinated variety.

As used herein, the term “orthologue” refers to a nucleic acid or peptide sequence which functions similarly to a nucleic acid or peptide sequence from another species.

As used herein, the term “ovule” refers to the female gametophyte, whereas the term “pollen” means the male gametophyte.

As used herein, the term “phenotype” refers to the observable characters of an individual cell, cell culture, plant, or group of plants which results from the interaction between that individual's genetic makeup (i.e., genotype) and the environment.

As used herein, the term “plant” refers to whole plants, plant organs (e.g., leaves, stems, roots, etc.), seeds and plant cells and progeny of it. The class of plants that can be used in the methods of the invention is generally as broad as the class of higher plants amenable to transformation techniques, including both monocotyledonous and dicotyledonous plants.

As used herein, the term “promoter” refers to a region of DNA involved in binding RNA polymerase to initiate transcription.

As used herein, the terms “protein,” “peptide” or polypeptide” refer to amino acid residues and polymers thereof. Unless specifically limited, the terms encompass amino acids containing known analogues of natural amino acid residues that have similar binding properties as the reference amino acid and are metabolized in a manner similar to naturally occurring amino acid residues. Unless otherwise indicated, a particular amino acid sequence also implicitly encompasses conservatively modified variants thereof (e.g. conservative substitutions) as well as the sequence explicitly indicated. The term “polypeptide” also encompasses polypeptides synthesized in a laboratory using procedures well known to those skilled in the art.

As used herein, the term “recombinant” refers to a cell, tissue or organism that has undergone transformation with recombinant DNA. The original recombinant is designated as “R0” or “R₀.” Selfing the R0 produces a first transformed generation designated as “R1” or “R₁”.

As used herein, the term “self pollinated” or “self-pollination” means the pollen of one flower on one plant is applied (artificially or naturally) to the ovule (stigma) of the same or a different flower on the same plant.

As used herein, the term “synthetic” refers to a set of progenies derived by intercrossing a specific set of clones or seed-propagated lines. A synthetic may contain mixtures of seed resulting from cross-, self-, and sib-fertilization.

As used herein, the term “tansformation” refers to the transfer of nucleic acid (i.e., a nucleotide polymer) into a cell. As used herein, the term “genetic transformation” refers to the transfer and incorporation of DNA, especially recombinant DNA, into a cell.

As used herein, the term “transformant” refers to a cell, tissue or organism that has undergone transformation. The original transformant is designated as “T0” or “T₀.” Selfing the T0 produces a first transformed generation designated as “T1” or “T₁.”

As used herein, the term “transgene” refers to a nucleic acid that is inserted into an organism, host cell or vector in a manner that ensures its function.

As used herein, the term “transgenic” refers to cells, cell cultures, organisms, plants, and progeny of plants which have received a foreign or modified gene by one of the various methods of transformation, wherein the foreign or modified gene is from the same or different species than the species of the plant, or organism, receiving the foreign or modified gene.

As used herein, the term “transposition event” refers to the movement of a transposon from a donor site to a target site.

As used herein, the term “transposon” refers to a genetic element, including but not limited to segments of DNA or RNA that can move from one chromosomal site to another.

As used herein, the term “variety” refers to a subdivision of a species, consisting of a group of individuals within the species that are distinct in form or function from other similar arrays of individuals.

As used herein, the term “vector” refers broadly to any plasmid or virus encoding an exogenous nucleic acid. The term should also be construed to include non-plasmid and non-viral compounds which facilitate transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, a recombinant vaccinia virus, a recombinant adenovirus, a recombinant retrovirus, a recombinant adenb-associated virus, a recombinant avian pox virus, and the like (Cranage et al., 1986, EMBO J. 5:3057-3063; International Patent Application No. WO94/17810, published Aug. 18, 1994; International Patent Application No. WO94/23744, published Oct. 27, 1994). Examples of non-viral vectors include, but are not limited to, liposomes, polyamine derivatives of DNA, and the like.

I Nucleic Acids

A. Promoters

There are many excellent examples of suitable promoters to drive gene expression in plants. Promoters have been identified in many plant species such as maize, rice, tomato, tobacco, Arabidopsis, Brassica, and others (Odell, T. O., et al. (1985) Nature 313:810-812; Marrs, K. A., et al, (1993) Dev Genet, Vol. 14/1:27-41; Kim, (1992) Transgenic Res, Vol. 1/4:188-94; Carpenter, J. L., et al. (1992) Plant Cell Vol. 4/5:557-71; Albani, D. et al., (1992) Plant J. 2/3:331-42; Rommens, C. M., et al. (1992), Mol. Gen. Genet., Vol. 231/3:433-41; Kloeckener-Gruissem, et al., (1992) Embo J, Vol. 11/1:157-66; Hamilton, D. A. et al., (1992), Plant Mol Biol, Vol. 18/2:211-18; Kyozuka, J., et al. (1991), Mol. Gen. Genet., Vol. 228/1-2:40-8; Albani, D. et al., (1991) Plant Mol Biol Vol. 16/4:501-13; Twell, D. et al. (1991) Genes Dev. 5/3:496-507; Thorsness, M. K. et al., (1991) Dev. Biol Vol. 143/1:173-84; McCormick, S. et al. (1991) Symp Soc Exp Biol Vol. 45:229-44; Guerrero, F. D. et al. (1990) Mol Gen Genet Vol 224/2:161-8; Twell, D. et al., (1990) Development Vol. 109/3:705-13; Bichler, J. et al. (1990), Eur J Biochem Vol. 190/2:415-26; van Tunen, et al. (1990), Plant Cell Vol 2/5:393-401; Siebertz, B. et al., (1989) Plant Cell Vol 1/10:961-8; Sullivan, T. D. et al., (1989) Dev Genet Vol 10/6:412-24; Chen, J. et al. (1987), Genetics Vol 116/3:469-77). Additional promoters can be found in GenBank. The CaMV 35S promoter is provided in U.S. Pat. Nos. 5,034,322; 5,086,169; 5,756,324; 5,633,438; 5,412,085; 5,545,546; 6,172,279 and 6,174,724.

B. Transgenes and Heterologous Nucleic Acids

There are numerous examples of genes successfully introduced into plants using recombinant DNA methodologies including, but not limited to, those coding for the following traits: seed storage proteins, including modified 7S legume seed storage proteins (U.S. Pat. Nos. 5,508,468, 5,559,223 and 5,576,203); herbicide tolerance or resistance (U.S. Pat. Nos. 5,498,544 and 5,554,798; Powell et al., Science 232:738-743 (1986); Kaniewski et al., Bio/Tech. 8:750-754 (1990); Day et al., Proc. Natl. Acad. Sci. USA 88:6721-6725 (1991)); phytase (U.S. Pat. No. 5,593,963); resistance to bacterial, fingal, nematode and insect pests, including resistance to the lepidoptera insects conferred by the Bt gene (U.S. Pat. Nos. 5,597,945 and 5,597,946; Hilder et al., Nature 330:160-163; Johnson et al., Proc. Natl. Acad. Sci. USA, 86:9871-9875 (1989); Perlak et al., Bio/Tech. 8:939-943 (1990)); lectins (U.S. Pat. No. 5,276,269); and flower color (Meyer et al., Nature 330:677-678 (1987); Napoli et al., Plant Cell 2:279-289 (1990); van der Krol et al., Plant Cell 2:291-299 (1990)).

C. Site-Specific Recombination Systems

Methods and constructs for targeting of DNA sequences for insertion into a particular DNA locus, while enabling removal of randomly inserted DNA sequences that occur as a by-product of transformation procedures, are described in U.S. Pat. Nos. 5,527,695 and 6,114,600. One manner of removing these random insertions is to utilize a site-specific recombinase system. In general, a site-specific recombinase system consists of three elements: two pairs of DNA sequence (the site-specific recombination sequences) and a specific enzyme (the site-specific recombinase). The site-specific recombinase will catalyze a recombination reaction only between two site-specific recombination sequences.

A number of different site-specific recombinase systems can be used, including but not limited to the Cre/lox system of bacteriophage P1, the FLP/FRT system of yeast, the Gin recombinase of phage Mu, the Pin recombinase of E. coli, and the R/RS system of the pSR1 plasmid. The two preferred site-specific recombinase systems are the bacteriophage P1 Cre/lox and the yeast FLP/FRT systems. In these systems a recombinase (Cre or FLP) will interact specifically with its respective site-specific recombination sequence (lox or FRT respectively) to invert or excise the intervening sequences. The sequence for each of these two systems is relatively short (34 bp for lox and 47 bp for FRT). Currently the FLP/FRT system of yeast is the preferred site-specific recombinase system since it normally functions in a eukaryotic organism (yeast), and is well characterized. It is thought that the eukaryotic origin of the FLP/FRT system allows the FLP/FRT system to function more efficiently in eukaryotic cells than the prokaryotic site-specific recombinase systems.

The FLP/FRT recombinase system has been demonstrated to function efficiently in plant cells. Experiments on the performance of the FLP/FRT system in both maize and rice protoplasts indicates that FRT site structure, and amount of the FLP protein present, affects excision activity. In general, short incomplete FRT sites leads to higher accumulation of excision products than the complete full-length FRT sites. Site-specific recombination systems can catalyze both intra- and intermolecular reactions in maize protoplasts, indicating that the system can be used for DNA excision as well as integration reactions. The recombination reaction is reversible and this reversibility can compromise the efficiency of the reaction in each direction. Altering the structure of the site-specific recombination sequences is one approach to remedying this situation. The site-specific recombination sequence can be mutated in a manner that the product of the recombination reaction is no longer recognized as a substrate for the reverse reaction, thereby stabilizing the integration or excision event.

D. Vector

### Expression Units to Express Exogenorus DN a Plant

As provided above, several embodiments of the present invention employ expression units (or expression vectors or systems) to express an exogenously supplied nucleic acid sequence in a plant. Methods for generating expression units/systems/vectors for use in plants are well known in the art and can readily be adapted for use in the instant invention. A skilled artisan can readily use any appropriate plant/vector/expression system in the present methods following the outline provided herein.

The expression control elements used to regulate the expression of the protein can either be the expression control element that is normally found associated with the coding sequence (homologous expression element) or can be a heterologous expression control element. A variety of homologous and heterologous expression control elements are known in the art and can readily be used to make expression units for use in the present invention. Transcription initiation regions, for example, can include any of the various opine initiation regions, such as octopine, mannopine, nopaline and the like that are found in the Ti plasmids of Agrobacterium tumafacians. Alternatively, plant viral promoters can also be used, such as the cauliflower mosaic virus 19S and 35S promoters (CaMV 19S and CaMV 35S promoters, respectively) to control gene expression in a plant (U.S. Pat. Nos. 5,352,605; 5,530,196 and 5,858,742 for example). Enhancer sequences derived from the CaMV can also be utilized (U.S. Pat. Nos. 5,164,316; 5,196,525; 5,322,938; 5,530,196; 5,352,605; 5,359,142; and 5,858,742 for example). Lastly, plant promoters such as prolifera promoter, fruit-specific promoters, Ap3 promoter, heat shock promoters, seed-specific promoters, etc. can also be used.

Either a gamete-specific promoter, a constitutive promoter (such as the CaMV or Nos promoter), an organ-specific promoter (such as the E8 proinoter from tomato) or an inducible promoter is typically ligated to the protein or antisense encoding region using standard techniques known in the art. The expression unit may be further optimized by employing supplemental elements such as transcription terminators and/or enhancer elements.

Thus, for expression in plants, the expression units will typically contain, in addition to the protein sequence, a plant promoter region, a transcription initiation site and a transcription termination sequence. Unique restriction enzyme sites at the 5′ and 3′ ends of the expression unit are typically included to allow for easy insertion into a preexisting vector.

In the construction of heterologous promoter/structural gene or antisense combinations, the promoter is preferably positioned about the same distance from the heterologous transcription start site as it is from the transcription start site in its natural setting. As is known in the art, however, some variation in this distance can be accommodated without loss of promoter function.

In addition to a promoter sequence, the expression cassette can also contain a transcription termination region downstream of the structural gene to provide for efficient termination. The termination region may be obtained from the same gene as the promoter sequence or may be obtained from different genes. If the mRNA encoded by the structural gene is to be efficiently processed, DNA sequences which direct polyadenylation of the RNA are also commonly added to the vector construct. Polyadenylation sequences include, but are not limited to the Agrobacterium octopine synthase signal (Gielen et al., EMBO J 3:835-846 (1984)) or the nopaline synthase signal (Depicker et al., Mol. and Appl. Genet 1:561-573 (1982)).

The resulting expression unit is ligated into or otherwise constructed to be included in a vector that is appropriate for higher plant transformation. The vector will also typically contain a selectable marker gene by which transformed plant cells can be identified in culture. Usually, the marker gene will encode antibiotic resistance. These markers include resistance to G418, hygromycin, bleomycin, kanamycin, and gentamicin: After transforming the plant cells, those cells having the vector will be identified by their ability to grow on a medium containing the particular antibiotic. Replication sequences, of bacterial or viral origin, are generally also included to allow the vector to be cloned in a bacterial or phage host, preferably a broad host range prokaryotic origin of replication is included. A selectable marker for bacteria should also be included to allow selection of bacterial cells bearing the desired construct. Suitable prokaryotic selectable markers also include resistance to antibiotics such as ampicillin, kanamycin or tetracycline.

Other DNA sequences encoding additional functions may also be present in the vector, as is known in the art. For instance, in the case of Agrobacterium transformations, T-DNA sequences will also be included for subsequent transfer to plant chromosomes.

The sequences of the present invention can also be fused to various other nucleic acid molecules such as Expressed Sequence Tags (ESTs), epitopes or fluorescent protein markers.

ESTs are gene fragments, typically 300 to 400 nucleotides in length, sequenced from the 3′ or 5′ end of complementary-DNA (cDNA) clones. Nearly 30,000 Arabidopsis thaliana ESTs have been produced by a French and an American consortium (Delseny et al., FEBS Lett. 405(2):129-132 (1997); Arabidopsis thaliana Database, http://genome.www.stanford.edu/Arabidopsis). For a discussion of the analysis of gene-expression patterns derived from large EST databases, see, e.g., M. R. Fannon, TIBTECH 14:294-298 (1996).

Biologically compatible fluorescent protein probes, particularly the self-assembling green fluorescent protein (GFP) from the jellyfish Aequorea victoria, have revolutionized research in cell, molecular and developmental biology because they allow visualization of biochemical events in living cells (Murphy et al., Curr. Biol. 7(11):870-876 (1997); Grebenok et al., Plant J. 11(3):573-586 (1997); Pang et al., Plant Physiol 112(3) (1996); Chiu et al., Curr. Biol. 6(3):325-330 (1996); Plautz et al., Gene 173(1):83-87 (1996); Sheen et al., Plant J. 8(5):777-784 (1995)).

Site-directed mutagenesis has been used to develop a more soluble version of the codon-modified GFP called soluble-modified GFP (smGFP). When introduced into Arabidopsis, greater fluorescence was observed when compared to the codon-modified GFP, implying that smGFP is ‘brighter’ because more of it is present in a soluble and functional form (Davis et al., Plant Mol. Biol. 36(4):521-528 (1998)). By fusing genes encoding GFP and beta-glucuronidase (GUS), researchers were able to create a set of bifunctional reporter constructs which are optimized for use in transient and stable expression systems in plants, including Arabidopsis (Quaedvlieg et al., Plant Mol. Biol. 37(4):715-727 (1998)).

Berger et al. (Dev. Biol. 194(2):226-234 (1998)) report the isolation of a GFP marker line for Arabidopsis hypocotyl epidermal cells. GFP-fusion proteins have been used to localize and characterize a number of Arabidopsis genes, including geranylgeranyl pyrophosphate (GGPP) (Zhu et al., Plant Mol. Biol. 35(3):331-341 (1997).

II. Transformation

A. Plant Transformation

To introduce a desired gene or set of genes by conventional methods requires a sexual cross between two lines, and then repeated back-crossing between hybrid offspring and one of the parents until a plant with the desired characteristics is obtained. This process, however, is restricted to plants that can sexually hybridize, and genes in addition to the desired gene will be transferred.

Recombinant DNA techniques allow plant researchers to circumvent these limitations by enabling plant geneticists to identify and clone specific genes for desirable traits, such as resistance to an insect pest, and to introduce these genes into already useful varieties of plants. Once the foreign genes have been introduced into a plant, that plant can than be used in conventional plant breeding schemes (e.g., pedigree breeding, single-seed-descent breeding schemes, reciprocal recurrent selection) to produce progeny which also contain the gene of interest.

Genes can be introduced in a site directed fashion using homologous recombination. Homologous recombination permits site-specific modifications in endogenous genes and thus inherited or acquired mutations may be corrected, and/or novel alterations may be engineered into the genome.

Homologous recombination and site-directed integration in plants are discussed above and in, for example, U.S. Pat. Nos. 5,451,513; 5,501,967 and 5,527,695.

Genetic manipulation methods can be used to produce transformed cells, tissues and whole plants expressing/over-expressing one or more ysl and/or ysl nucleic acids of the present invention. For bioremediation efforts, the transformed plants can be grown on soils with high metal or heavy metal content and then harvested, thereby removing metals or heavy metals accumulated in the harvested plant parts. Preferable plants to transform for bioremediation purposes include those with rapid growth characteristics, high biomass production and extensive, highly branched root systems. Particularly preferable plants of this type include, but are not limited to the forage grass plants, especially the Festuca species; herbs; shrubs; and woody plants such as Liriodendron tulipifera (yellow-poplar) and Serbertia, Shorea and Myristica species. Other preferable plants to transform using the ysl and ysl nucleic acids of the present invention are the hyperaccumulator plants, especially plants of the Brassica species.

For nutritive purposes, the transformed plants to be grown can include those that are consumed by humans or animals, either directly or in processed food products. For example, transformed plants can be produced that accumulate metals or heavy metals in the whole plant or in one or more specific plant parts, such as in the kernel, tuber, fruit or seed. Preferable plants to transform using the nucleic acids of the present invention include plants that are widely grown for human consumption, such as rice, soybeans, wheat, oat, rye, cassava, potatoes, green beans, dry peas, lentils, strawberries, oranges and the like. Consumption of the transformed plants or plant parts can improve the value of the food consumed by the organism as regards specific heavy metals. The transformed plants can be grown in any media that has low, average or high content and/or concentrations of one or more metals or heavy metals. Plant species that are useful for both bioremediation and nutritive purposes can also be used. For example, transformed forage species may be effective for phytoremediation and may also be useful as livestock feed. The transformed forage can be consumed by grazing animals or can be cut and dried to produce hay for animal feed. Examples of transformed plants useful as animal feeds include, but are not limited to, alfalfa, clover and various grass species used as forages.

B. Transformation Methods

Methods of producing transgenic plants are well known to those of ordinary skill in the art. Transgenic plants can now be produced by a variety of different transformation methods including, but not limited to, electroporation; microinjection; microprojectile bombardment, also known as particle acceleration or biolistic bombardment; viral-mediated transformation; and Agrobacterium-mediated transformation (see, e.g., U.S. Pat. Nos. 5,405,765; 5,472,869; 5,538,877; 5,538,880; 5,550,318; 5,641,664; 5,736,369 and 5,736369; Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., Bio/Tech. 8:833-839 (1990); Mullins et al., Bio/Tech. 8:833-839 (1990); and, Raineri et al., Bio/Tech. 8:33-38 (1990)).

Transgenic alfalfa plants have been produced by many of these methods including, but not limited to, agrobacterium-mediated transformation (Wang et al., Australian Journal of Plant Physiology 23(3):265-270 (1996); Hoffman et al., Molecular Plant-Microbe Interactions 10(3):307-315 (1997); Trieu et al., Plant Cell Reports 16:6-11 (1996)) and particle acceleration (U.S. Pat. No. 5,324,646).

Transformation has also been successfully accomplished in clover using agrobacterium-mediated transformation (Voisey et al., Biocontrol Science and Technology 4(4):475-481 (1994); Quesbenberry et al., Crop Science 36(4):1045-1048(1996); Khan et al., Plant Physiology 105(1):81-88 (1994); Voisey et al., Plant Cell Reports 13(6):309-314 (1994)).

Genetic transformation has also been reported in numerous forage and turfgrass species (Conger B. V. Genetic Transformation of Forage Grasses in Molecular and Cellular Technologies for Forage Improvement, CSSA Special Publication No. 26, Crop Science Society of America, Inc. E. C. Brummer et al. Eds. 1998, pages 49-58). These include orchardgrass (Dactylis glomerata L.), tall fescue (Festuca arundinacea Schreb.) red fescue (Festuca rubra L.), meadow fescue (Festuca pratensis Huds.) perennial ryegrass (Lolium perenne L.) creeping bentgrass (Agrostis palustris Huds.) and redtop (Agrostis alba L.).

Successful gene transfer in such forages and turfgrasses has been accomplished by direct uptake of DNA by protoplasts and by bombardment of cells or tissues with DNA coated microprojectiles. In both cases, the transfer is followed by whole plant regeneration. Much of the work has focused on developing and improving protocols for the transformation and have used the reporter gene uidA coding for—glucouronidase (GUS) and the selectable marker bar that confers tolerance to phosphinothricin-based herbicides. Proof of the transformation has been provided by polymerase chain reaction (PCR) techniques, northern hybridization analysis of transcribed RNA, western blot analysis of soluble protein (gene product), and southern blot hybridization of total genomic DNA.

III. Hemizyosity

A transgenic plant formed using Agrobacterium transformation methods typically contains a single gene on one chromosome, although multiple copies are possible. Such transgenic plants can be referred to as being hemizygous for the added gene. A more accurate name for such a plant is an independent segregant, because each transformed plant represents a unique T-DNA integration event (U.S. Pat. No. 6,156,953). A transgene locus is generally characterized by the presence and/or absence of the transgene. A heterozygous genotype in which one allele corresponds to the absence of the transgene is also designated hemizygous (U.S. Pat. No. 6,008,437).

Assuming normal hemizygosity, selfing will result in maximum genotypic segregation in the first selfed recombinant generation, also known as the R1 or R₁ generation. The R1 generation is produced by selfing the original recombinant line, also known as the R0 or R₀ generation. Because each insert acts as a dominant allele, in the absence of linkage and assuming only one hemizygous insert is required for tolerance expression, one insert would segregate 3:1, two inserts, 15:1, three inserts, 63:1, etc. Therefore, relatively few R1 plants need to be grown to find at least one resistance phenotype (U.S. Pat. Nos. 5,436,175 and 5,776,760).

As mentioned above, self-pollination of a hemizygous transgenic regenerated plant should produce progeny equivalent to an F2 in which approximately 25% should be homozygous transgenic plants. Self-pollination and testcrossing of the F2 progeny to non-transformed control plants can be used to identify homozygous transgenic plants and to maintain the line. If the progeny initially obtained for a regenerated plant were from cross-pollination, then identification of homozygous transgenic plants will require an additional generation of self-pollination (U.S. Pat. No. 5,545,545).

IV. Breeding Methods

Open-Pollinated Populations. The improvement of open-pollinated populations of such crops as rye, many maizes and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes for flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are basically two primary methods of open-pollinated population improvement. First, there is the situation in which a population is changed en masse by a chosen selection procedure. The outcome is an improved population that is indefinitely propagable by random-mating within itself in isolation. Second, the synthetic variety attains the same end result as population improvement but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988).

Mass Selection. In mass selection, desirable individual plants are chosen, harvested, and the seed composited without progeny testing to produce the following generation. Since selection is based on the maternal parent only, and there is no control over pollination, mass selection amounts to a form of random mating with selection. As stated above, the purpose of mass selection is to increase the proportion of superior genotypes in the population.

Synthetics. A synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or topcrosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or two cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enter a synthetic vary widely. In practice, numbers of parental lines range from 10 to several hundred, with 100-200 being the average. Broad based synthetics formed from 100 or more clones would be expected to be more stable during seed multiplication than narrow based synthetics.

Hybrids. A hybrid is an individual plant resulting from a cross between parents of differing genotypes. Commercial hybrids are now used extensively in many crops, including corn (maize), sorghum, sugarbeet, sunflower and broccoli. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

Strictly speaking, most individuals in an out breeding (ie., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

The production of hybrids is a well-developed industry, involving the isolated production of both the parental lines and the hybrids which result from crossing those lines. For a detailed discussion of the hybrid production process, see, e.g., Wright, Commercial Hybrid Seed Production 8:161-176, In Hybridization of Crop Plants.

V. Expression Assays

The present invention further provides methods of recognizing variations in the DNA sequence of Zea mays ys1 and the Arabidopsis ysl1-8 in those species as well as for detecting the gene or its homologues or orthologues in other plant genera, species, strains, varieties or cultivars. One method involves the introduction of a nucleic acid molecule (also known as a probe or nucleic acid probe) having a sequence identical or complementary to at least a portion of at least one of the ys1 (SEQ ID NO: 1) or ysl1-8 sequences (SEQ ID NO: 3, 5, 7, 9, 11, 13, 15 or 17) of the invention under sufficient hybridizing conditions as would be understood by those in the art, such as the moderately stringent or highly stringent hybridization conditions as described elsewhere within the instant description. Said probe would share identity with the DNA sequence of SEQ ID NO: 1, 3, 5, 7, 9, 11, 13, 15 or 17 over at least about 10 contiguous nucleic acid residues. Preferably, said identity would be over at least about 25 or 30 contiguous nucleic acid residues. More preferably, said identity would be over at least about 40 or 50 contiguous nucleic acid residues. Even more preferably, said identity would be over at least about 60 or 75 contiguous nucleic acid residues. Still more preferably, said identity would be over at least about 100 or 150 contiguous nucleic acid residues. Yet more preferably, said identity would be over at least about 200 or 250 contiguous nucleic acid residues. Most preferably, said identity would be over at least about 300 contiguous nucleic acid residues or would math the entire open reading frame of SEQ ID NO: 1, 3, 5, 7, 9, 11, 13, 15 or 17 or its complement. Another method of recognizing DNA sequence variation is direct DNA sequence analysis by multiple methods well known in the art. Another embodiment involves the detection of DNA sequence variation in YS1 or YSL proteins as represented by different plant genera, species, strains, varieties or cultivars. Another embodiment involves using said nucleic acid probes for the detection of ys1 and/or ysl sequences in a sample or tissue section using in situ hybridization according to any method known to those of skill in the art. The ys1 or ysl sequence used for the probe can be from any plant for which the presence of ys1 or ysl has been determined. A particularly good probe for dicotyledonous plants would be that coding for one of YSL1-8 of Arabidopsis, while a particularly good probe for a monocotyledonous plant would be that coding for the YS1 of maize. In one embodiment, the sequence will bind specifically to one allele of a YS1 or YSL-encoding gene, or a fragment thereof, and in another embodiment will bind to multiple alleles. Such detection methods include the polymerase chain reaction, restriction fragment length polymorphism (RFLP) analysis and single stranded conformational analysis.

Diagnostic probes useful in such assays of the invention include antibodies to YS1 or one of the Arabidopsis YSL proteins. The antibodies to YS1 or at least one of YSL1-8 may be either monoclonal or polyclonal, produced using standard techniques well known in the art (See Harlow & Lane's Antibodies: A Laboratory Manual, Cold Spring Harbor Laboratory Press, 1988). They can be used to detect YS1 or a YSL, or a homologue or orthologue thereof, protein by binding to the protein and subsequent detection of the antibody-protein complex by ELISA, Western blot or the like. The YS1 or YSL sequence used to elicit these antibodies can be any of the YS1 or YSL variants discussed above. Antibodies are also produced from peptide sequences of YS1 or at least one of YSL1-8 using standard techniques in the art (See Protocols in Immunology, John Wiley & Sons, 1994). Fragments of the monoclonals or the polyclonal antisera which-contain the immunologically significant portion can also be prepared.

Assays to detect or measure YS1 or YSL polypeptide in a biological sample with an antibody probe may be based on any available format. For instance, in immunoassays where YS1 or YSL polypeptides are the analyte, the test sample, typically a biological sample, is incubated with anti-YS1 or anti-YSL antibodies under conditions that allow the formation of antigen-antibody complexes. Various formats can be employed, such as “sandwich” assay where antibody bound to a 'solid support is incubated with the test sample; washed, incubated with a second, labeled antibody to the analyte; and the support is washed again. Analyte is detected by determining if the second antibody is bound to the support. In a competitive format, which can be either heterogeneous or homogeneous, a test sample is usually incubated with an antibody and a labeled competing antigen, either sequentially or simultaneously. These and other formats are well known in the art. Alternatively, a test sample may be a tissue section of a plant which is probed with an antibody to YS1 and/or one or more of the YSL proteins using methods well known to those in the art for detection of proteins in a tissue section with an antibody. Said tissue section may be from a plant being tested for natural expression of YS1 and/or one or more of the YSL proteins or a homologue or orthologue thereof. Alternatively, said tissue section may be from a plant which has been genetically altered by the means of the present invention or by some other means to express at least one protein selected from the group consisting of YS1, YSL 1-8 and homologues or orthologues thereof.

### EXAMPLES

### Example 1. Transposon-Tagging and Cloning of ys1

The endogenous maize transposon Ac, located on chromosome 1 at the P1 locus (P-VV allele) was used in a random mutagenesis strategy as described (Dellaporta, SL et al. In The Maize Handbook. Eds. Freeling & Walbot. Springer, New York, 1993, pages 219-233). After screening for transposition events, seedlings were screened for mutant phenotypes, and a family segregating a yellow striped mutation at a frequency of approximately 25% was identified by visual observations. Genomic blotting was performed on individuals from a family segregating phenotypically for WT and mutant individuals using Ac as a probe. DNA from the parental strains, P-VV (the Ac-donor locus) and r-m3 were also included in the blotting tests. All samples were digested with restriction enzyme SalI. The probe was the internal HindIII fragment of Ac. The blots confirmed co-segregation of an Ac-containing SalI restriction fragment of 9.5 kb with the yellow striped mutant phenotype.

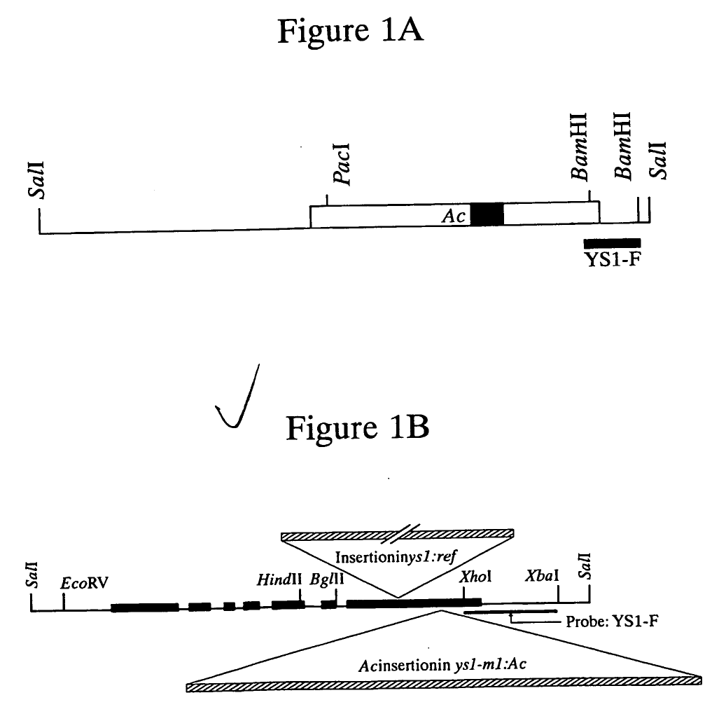

A genomic library was prepared from the DNA of a mutant plant, and a clone, λYS31, containing a 9.5 kb SalI insert was identified, shown in FIG. 1A.

An Ac-flanking probe that contains sequences adjacent to the Ac element (YS1-F; see FIG. 1A and 1B) was prepared from λYS3 1, and used as a probe on genomic blots of families segregating for the yellow stripe mutation. Genomic blots were performed on DNA of individuals from a family segregating phenotypically for WT and mutant individuals, as well as on the parental strains, P-VV (the Ac-donor locus) and r-m3. All samples were digested with restriction enzyme SalI. Each mutant individual showed the 9.5 kb SalI fragment, as did heterozygous wild type plants. One mutant plant showed a 5.2 kb SalI fragment that is the size expected following transposition of Ac from the 9.5 kb fragment. Notably, neither heterozygous nor homozygous WT plants showed the 5.2 kb SalI fragment expected. The lack of the 5.2 kb fragment is probably due to cytosine methylation of the SalI sites in the WT Ys1 allele. It appears that, upon Ac insertion, the locus became demethylated, and that the demethylated state persists for a time following Ac excision from the locus.

To confirm the co-segregation analysis, DNA was prepared from a second family that segregated the yellow stripe mutation, so that co-segregation in a new set of individuals could be tested. The DNA was digested with EcoRV, an enzyme that is insensitive to methylation. The blots were first probed with YS1 -F, and then stripped and re-probed with the Ac probe. On these blots, the smaller fragment (lacking Ac) co-segregated with the wild-type phenotype, as expected.

### Example 2. Linkage Analysis for ys1

Heterozygous Ys1 pr1/ys1-m1::Ac Pr1 plants were self-pollinated. Red (pr1/pr1) and purple (Pr1/-) progeny were selected, and their yellow stripe phenotype was observed. The red-colored progeny were predominately wild-type with respect to yellow stripe showing that there is a clear linkage between ys1 m1::Ac and pr1, as expected. Among the purple progeny, roughly one-third of the individuals were yellow stripped, again showing a clear linkage between ys1 m1::Ac and pr1.

Linkage and complementation assays were performed with the new yellow stripe mutant. Mutant plants were crossed to plants homozygous for the reference mutant alleles of ys1 (ys1-ref) and ys3 (ys3-ref) to test for complementation. The new yellow striped mutant failed to complement ys1, but did complement ys3, thus the new mutant is a ys1 allele designated ys1-m1::Ac. The ys1 locus was mapped on chromosome 5 of maize, 8 map units distal to the pr1 locus. Linkage of ys1-m1::Ac and pr1 was tested and confirmed that the new mutant is linked to pr1.

### Example 3. Cloning and Characterization of ys1 cDNA

The YS1-F probe was used to screen a root cDNA library from iron deficient maize plants (Loulergue, C et al. Gene. 1998 225:47-57). Three full-length or nearly full-length ys1 cDNAs were recovered. Although the precise sizes of the three cDNAs differed because of alternative polyadenylation sites and sizes of 5′ untranslated regions (UTRs), they all encoded identical proteins.

The sequence of these cDNAs indicates that the YS1 protein is 682 amino acids long and contains 12 putative transmembrane domains, thus YS1 is likely to be localized to the membrane, as would be expected if YS1 is a transporter for Fe.phytosiderophore complexes.

The predicted amino acid sequence of YS1 is as follows, with the 12 putative membrane-spanning domains predicted using the SOSUI program shown underlined:

| | |
|---|---|
| MDLARRGGAAGADDEGEIERHEPAPEDMESDPAAAR | (SEQ ID NO: 2) |
| EKELELERVQSWREQVTLRGVVAALLIGFMYSVIVM | |
| KIALTTGLVPTLNVSAALMAFLALRGWTRVLERLGV | |
| AHRPFTRQENCVIETCAVACYTIAFGGGFGSTLLGL | |
| DKKTYELAGASPANVPGSYKDPGFGWMAGFVAAISF | |
| AGLLSLIPLRKVLVIDYKLTYPSGTATAVLINGFHT | |
| KQGDKNARMQVRGFLKYFGLSFVWSFFQWFYTGGEV | |
| CGFVQFPTFGLKAWKQTFFFDFSLTYVGAGMICSHL | |
| VNISTLLGAILSWGILWPLISKQKGEWYPANIPESS | |
| MKSLYGYKAFLCIALIMGDGTYHFFKVFGVTVKSLH | |
| QRLSRKRATNRVANGGDEMAALDDLQRDEIFSDGSF | |
| PAWAAYAGYAALTVVSAVIIPHMFRQVKWYYVIVAY | |
| VLAPLLGFANSYGTGLTDINMAYNYGKIALFIFAAW | |
| AGRDNGVIAGLAGGTLVKQLVMASADLMHDFKTGHL | |
| TMTSPRSLLVAQFIGTAMGCVVAPLTFLLFYNAFDI | |
| GNPTGYWKAPYGLIYRNMAILGVEGFSVLPRHCLAL | |
| SAGFFAFAFVFSVARDVLPRKYARFVPLPMAMAVPF | |
| LVGGSFAIDMCVGSLAVFVWEKVNRKEAVFMVPAVA | |
| SGLICGDGIWTFPSSILALAKIKPPICMKFTPGS | |

Notably, the 50 amino-terminal amino acids of YS1 contain 48% of the glutamic-acid residues of the protein (11 out of 23). Some of these are in the sequence REKELELELER (SEQ ID NO: 19) which is reminiscent of the REGLE (SEQ ID NO: 20) sequence involved in Fe[III] transport (Stearman, R et al. Science 1996 271:1552-1557).

The amino-acid sequence from the ys1 cDNA does not show strong sequence similarity to any protein with known function in the various sequence databases, but it shows similarity expressed sequence tag (EST) clones in diverse plant species including both monocots and dicots, gymnosperms and mosses. YS1 also shows similarity to a hypothetical yeast protein, YGL114 (36% positive; GenBank accession number P53134), belonging to the major facilitator superfamily (MFS; Pao, SS et al. Microbiol. Mol. Biol. Rev. 1998 62:1-34), which includes single-polypeptide secondary carriers that typically transport small solutes in response to chemiosmotic ion gradients, and the EspB gene of Myxococcus xanthus (39% positive; GenBank accession number AAD47813.1). YS1 also belongs to a gene family in maize, as there are three related maize ESTs present in GenBank.

The amino acid sequence of YS1 also showed strong, full length similarity to eight predicted Arabidopsis proteins which we have designated YELLOW STRIPE1-LIKE (YSL) 1-8 (SEQ ID NO: 4, 6, 8, 10, 12, 14, 16 and 18, respectively). Notably, the abundance of glutamic acid residues at the amino terminus of YS1 is conserved among the eight Arabidopsis YS1-like homologs. YS1 is 73% identical over 665 amino acid residues to YSL1 (SEQ ID NO: 4), 77% identical over 658 amino acid residues to YSL2 (SEQ ID NO: 6), 76% identical over 668 amino acid residues to YSL3 (SEQ ID NO: 8), 69% identical over 644 amino acid residues to YSL4 (SEQ ID NO: 10), 67% identical over 680 amino acid residues to YSL5 (SEQ ID NO: 12), 70% identical over 604 amino acid residues to YSL6 (SEQ ID NO: 14), 69% identical over 674 amino acid residues to YSL7 (SEQ ID NO: 16) and 67% identical over 454 amino acid residues to YSL8 (SEQ ID NO: 18). The Arabidopsis ysl cDNA clones and their protein products are noted in Table 1.

TABLE 1
Arabidopsis Yellow Stripe1-Like
cDNA Clones & Proteins

| Name | BAC Clone | Old Protein ID | New Protein ID |
|---|---|---|---|
| YSL1 | T19F6 | T19F6.110 | At4g24120 |
| YSL2 | K16H17 | K16H17.9 | At5g24380 |
| YSL3 | MNC6 | MNC6.9 | At5g53550 |
| YSL4 | MEE6 | MEE6.7 | At5g41000 |
| YSL5 | MKP6 | MKP6.21 | At3g17650 |
| YSL6 | M0J10 | M0J10.9 | At3g27020 |
| YSL7 | F1E22 | F1E22.10 | At1g65730 |
| YSL8 | F11A17 | F11A17.8 | At1g48370 |

The cDNA clone of ysl1 is 2196 nucleic acid residues in length (SEQ ID NO: 3), having an open reading frame extending-from residue 10 to residue 2026, excluding the stop codon (2029 with the stop codon), and encodes a protein which is 673 amino acid residues in length (SEQ ID NO: 4). The cDNA clone of ysl2 is 2316 nucleic acid residues in length (SEQ ID NO: 5), having an open reading frame extending from residue 156 to residue 2145 (2148), and encodes a protein which is 664 amino acid residues in length (SEQ ID NO: 6). The cDNA clone of ysl3 maps to GenBank accession number (SEQ ID NO: 7) and is predicted to encode a protein of 675 amino acid residues in length (SEQ ID NO: 8). The cDNA clone of ysl4 maps to GenBank accession number (SEQ ID NO: 9) and is predicted to encode a protein of 670 amino acid residues in length (SEQ ID NO: 10). The cDNA clone of ysl5 is 2337 nucleic acid residues in length (SEQ ID NO: 11), having an open reading frame extending from residue 80 to residue 2221 (2224), and encodes a protein which is 714 amino acid residues in length (SEQ ID NO: 12). The cDNA clone of ysl6 is 2327 nucleic acid residues in length (SEQ ID NO: 13), having an open reading frame extending from residue 42 to residue 2072 (2075), and encodes a protein which is 677 amino acid residues in length (SEQ ID NO: 14). The cDNA clone of ysl7 is 2344 nucleic acid residues in length (SEQ ID NO: 15), having an open reading frame extending from residue 112 to residue 2175 (2178), and encodes a protein which is 688 amino acid residues in length (SEQ ID NO: 16). The cDNA clone of ysl8 is 2311 nucleic acid residues in length (SEQ ID NO: 17), having an open reading frame extending from residue 49 to residue 2220 (2223), and encodes a protein which is 724 amino acid residues in length (SEQ ID NO: 18).

### Example 4. Molecular Characterization of the Mutation in the Original Ys1 Mutant

The sequence of the ys1-m1::Ac genomic clone λYS31 was determined in the regions flanking the Ac insertion. Ac created an 8 bp target site duplication upon insertion, as expected. Ac is inserted within the coding region at amino acid position 649 relative to the start of translation.

The Ys1 wild type and ys1-ref alleles were amplified from genomic DNA using primers selected based on the cDNA sequence. Genomic blot analysis combined with polymerase chain reaction (PCR) of the corresponding genomic region indicates that the ys1-ref allele has a large insertion at amino-acid position 472 relative to the start of translation (see sequence above). Analysis of the ends of the inserted sequence indicates that it is a long-terminal repeat retrotransposon (data not shown).

Two additional ys1 mutant alleles, ys1:74-1924-1 and ys1:5344, were amplified and sequenced. The ys1:74-1924-1 mutation corresponds to a single nucleotide insertion that causes a frameshift altering the carboxy-terminal third of the protein sequence. The ys1:5344 allele has a slightly more complicated mutation involving a 16-base-pair (bp) deletion accompanied by a 2-bp insertion that causes a frameshift starting in the last transmembrane domain of the protein. The ys1-ref allele bears an insertion of 2 kb relative to wild type Ys1. Sequence analysis of this product indicates that this insertion is likely to be an LTR retrotransposon, since it contains a putative reverse transcripts coding region, and contains long terminal repeats and target site duplication characteristic of this type of element (data not shown). The position of this insertion within the ys1-ref allele is also within a coding region at amino acid number 474 relative to the start of translation. The sequence disruption in these additional ys1 mutant alleles and in the ys1:ref allele provides the final confirmation that we have cloned the ys1 gene.

### Example 5. Yeast Functional Complementation: Expression of ys1 cDNA Complements Iron Transport Defect in Yeast fet3fet4 Strain

Saccharomyces cerevisiae double mutant fet3fet4 (strain DEY1453) is defective in both low and high affinity iron (II) uptake systems and cannot grow on iron-limited medium (Bide, D et al. Proc. Natl. Acad. Sci. USA 1996 93:5624-5628), and cannot use iron complexed with the maize phytosiderophore deoxymugineic acid (Fe-DMA) for growth (Loulergue, C. Gene 1998 225:47-57). To investigate the function of YS1 in iron transport, we tested whether expression of ys1 cDNA could restore growth of the fet3fet4 mutant on medium containing Fe-DMA as the sole iron source.

Three plasmids were individually introduced into the DEY1453 (fet3fet4) strain: (1) ys1 cDNA cloned in the expression vector pYPGE15; (2) Arabidopsis IRT1 cDNA cloned in the pFL61 vector (Minet, M et al. 1992 Plant J. 2:417-422; and, as a control, (3) empty pYPGE15 vector. The IRT1 cDNA encodes an Arabidopsis thaliana iron transporter protein capable of supporting growth of the DEY 1453 strain on iron citrate. The ys1 and IRT1 cDNAs were both under the control of the strong PGK promoter (Loulergue, C et al. 1998 Gene 225:47-57). We then performed a differential growth test using two different sources of iron in the medium, Fe-citrate or Fe-DMA, both at low concentrations, to determine the substrate specificity, if any, of YS1. Yeast growth was on minimal medium/Ura supplemented with 5 μM Fe-citrate, 5 μM Fe-DMA, or 5 μM Fe-DMA and 5 μM BPDS. The Fe-DMA complex was prepared according to vonWiren, N et al. 1998 Biochem. Biophys. Acta 1372:143-155. Growth was carried out for 4 days at 30° C. Three yeast dilutions of the culture (of optical density at 600 nm of 0.2, 0.02 and 0.002) were spotted onto plates.

Expression of IRT1 restored growth of fet3fet4 when Fe-citrate was provided as sole iron source, as expected, whereas expression of YS1 did not. In the presence 5 μM Fe-DMA, both YS1 and IRT1 expression allowed growth of fet3fet4 mutant, possibly owing to small amounts of residual un-chelated Fe(II) present in the medium. The fact that ys1 complements the growth defect of fet3fet4 when iron is provided as Fe-DMA chelate, but not when iron is provided as Fe-citrate, suggests that ys1 encodes an iron transporter specific for Fe-DMA. To clarify this, the Fe-DMA medium was supplemented with 5 μM BPDS, a strong Fe(II) chelator, to remove any residual Fe(II) from the Fe-DMA medium. Addition of BPDS eliminated complementation by IRT1, without affecting complementation by YS1. The ability of YS1 to allow growth on Fe-DMA in the presence of BPDS strongly suggests that YS1 is a transporter of phytosiderophore-bound Fe(III).

### Example 6. Regulation of ys1 mRNA Levels by Iron Availability in Young Maize Plantlets

The effect of Fe starvation on ysl gene expression in maize was analyzed using Northern blot hybridization. Plants were grown hydroponically in presence (+) or absence (−) or iron, for 1, 5, 7 or 10 days after germination (Thoiron, S. et al. 1997 Plant Cell Env. 20:1051-1060). A 3′ UTR ys1 probe, obtained by PCR, was hybridized to a Northern blot containing 10 μg total RNA prepared from the roots of 1-, 5- and 7-day old plantlets and from the roots and shoots of 10-day-old plantlets. RNA extraction and RNA blot analysis were performed as described by Loulergue, C et al. 1998 Gene 225:47-57. Blots were stripped and hybridized to a NADPH-ferric (NRF) cDNA encoding a rice cytochrome b₅ reductase (Bagnaresi, P et al. Biochem. J. 1999 338:499-505). Ethidium-bromide-stained rRNAs were also obtained. Hybridization signals were revealed after 3 days exposure, using a PhosphorImager (Storm 480, Molecular Dynamics).

Expression of ys1 was detected in roots of young maize plantlets, as early as 1 day after germination. Abundance of ys1 mRNA increased several fold when plants were grown in absence of iron. The same induction was observed at 5, 7 and 10 days after germination, showing that steady-state levels of ys1 mRNA are increased by iron starvation in maize roots. This result agrees well with physiological studies in which maize plants grown under iron-sufficient conditions show a low, basal level of iron uptake, and show a 2.8-fold increase in the rate of iron uptake in conditions of iron deficiency (von Wiren, N. et al. Physiol. Plant 1995 95:611-616).

Expression of ys1 in leaves was investigated in 1 0-day-old plants grown in presence (+) or in absence (−) of iron. Roots of 10 day old iron-starved plants expressed higher levels of ys1 than roots of iron-sufficient plants. We did not detect ysl mRNA in leaves of iron-sufficient plants, but a high level of accumulation was detected in leaves of iron-deficient plants. It is possible that DMA serves as an iron carrier that transports iron from cell to cell inside the plant. Indeed, DMA has been detected in leaves of rice plants (Mori, S et al. 1991 Plant Soil 20 130:143-156). Alternatively, nicotianamine, a Fe(II) and Fe(III) chelator structurally related to DMA (von Wiren, N et al. Plant Physi6l. 1999 119:1107-1114), might be a substrate for transport by YS1 in tissues other than the root. Nicotianamine is found in all plant species, not just grasses, and has been proposed to be involved in long distance Fe(II) transport in the phloem sap (von Wiren, N et al. Plant Physiol. 1999 119:1107-1114; Stephan, UW et al. Plant Soil. 1994 165:181-188; Stephan, UW et al. Biometals 1996 9:8490). In that regard, we note that the YSL genes of Arabidopsis, a species which produces nicotianamine but not mugineic acids, might have a transport role similar to that of YS1.

### Example 7. YS1 Uptake of Cu in Corn

The ability of YS1 to transport Cu was also studied. Corn plantlets were grown in Cu-deficient soil for 10 days. Copper was then applied to the soil in the form of Cu-nicotinamine or Cu-phytosiderophore. RNA was extracted from roots and leaves of the plantlets and subjected to RNA blot analysis, as described in Example 6.

It was found that ys1 mRNA expression was increased in both roots and leaves in response to a lack of Cu. In addition, Southern blot analysis confirmed that there was an increase in YS1 protein expression in the roots as well.

### Example 8. YS1 Complements Cu Uptake in Deficient Yeast

In order to complement the findings in plants, the inventors performed an analysis of the ability of YS1 to complement yeast strains that are deficient in the uptake of Cu.

Consistent with the findings of Example 5, Cu-uptake-defective yeast mutants transformed with ys1 and grown on a Cu-supplemented medium were able to proliferate. Therefore, the ability of YS1 to transport some types of heavy metals in addition to iron has been confirmed.

These results demonstrate that, in relation to bioremediation, YS1 transgenic plants are useful not only for reducing the iron content of soils, but also for reducing copper levels in soil. The use of YS1 transgenic plants, therefore, will allow the reclamation of soils contaminated with copper as well with iron.

### Example 9. Iron Uptake by Arabidopsis YSL2 Protein

Similar to Example 4 above, Saccharomyces cerevisiae double mutant fet3fet4 (strain DEY1453) was used to investigate the function of YSL2 (SEQ ID NO: 5) in iron transport. We tested whether expression of ysl2 cDNA could restore growth of the fet3fet4 mutant on medium containing Fe-nicotinamide or Fe-citrate as the sole iron source. YSL2 was able to facilitate fet3fet4 growth on Fe-nicotinamide medium, but not on Fe-citrate medium. This confirms that YSL2 is a bonafide Fe-nicotinamide transporter.

### Example 10. Transgenic Plants for Increased Uptake of Nutritional Iron

Transgenic plants are engineered to enhance their ability to uptake iron from soil which is deficient in iron content, or where iron uptake is inhibited by high soil pH (alkalinity), high lime content, calcareous soil, excess phosphates in the soil, irrigation water containing high levels of bicarbonate ions, excess moisture along with low soil temperatures or any other condition which may interfere with a plant's ability to uptake iron from the soil. Engineering plants to enhance their ability to uptake iron increases the bioavailability of nutritional in the edible plant matter, better plant growth and/or increased crop yield.

Vectors comprising at least one of ys1 and/or ysl1-8 and a promoter which upregulates the expression of the gene under any condition which may interfere with a plant's ability to uptake iron from the soil are constructed with flanking sequences that allow their incorporation into the genome of any food crop plant. Transformed and WT seedlings are grown on soil media exemplary of various conditions of low iron bioavailability. Cultivars are selected that accumulate in their tissues a greater percentage of iron in their dry biomass than the wild-type controls.

For example, seedlings of transformed soybean or cassava can be grown side-by-side with parental wild-type plants in a sand/Perlite mixture that has been formulated to approximate a condition of low iron bioavailability, e.g., low soil iron concentration or high lime concentration. All plants are watered and given Hoagland nutrient solution, minus iron, regularly. Plants are allowed to grow to full harvest maturity and are then dried. Total plant iron concentration and iron concentration in the edible portions of the plant are assayed. Transformed plants demonstrating higher levels of iron accumulation than parental WT plants are selected for further propagation and, possibly, breeding programs using methods well known to those skilled in the art of plant breeding, plant selection and plant production.

### Example 11. Extraction of Metals from Soil

A number of plant species have been identified which are hyperaccumulators, meaning that they are capable of accumulating high levels of metals in their roots and other tissues without the metal being toxic to the plant when compared to WT plants grown under the same conditions. However, many of these plants are incapable of extracting heavy metal from soil without the addition of chelating agents to the soil. Accordingly, it is desirable to obtain hyperaccumulator plants that express at least one of maize YS1 and/or Arabidopsis YSL1-8 gene products and that will allow its growth and harvesting on metal contaminated soils without the constant need for applying chemical chelating agents to the soil.

Vectors comprising at least one of ys1 and/or ysl1-8 and a promoter that allows the expression of the gene under condition of high metal concentration in the soil are constructed with flanking sequences that allow their incorporation into the genome of any hyperaccumulator plant. Transformed and WT seedlings of, for example, Brassica juncea and Amaranthus paniculata are grown on soil media exemplary of conditions of heavy metal contamination of interest. Cultivars are selected that accumulate in their tissues a greater percentage of a given heavy metal in their dry biomass than the wild-type controls.

Seedlings of the WT and transformed Brassica juncea and Amaranthus paniculata can be planted in a sand/Perlite mixture and allowed to grow for 21 days. Then, solutions containing different concentrations of various metals with/without chelating agents (e.g., HEDTA, EDTA) are added to the soil. Between 2-500 micrograms of metal/gram soil can be applied. Plants are then watered and given Hoagland nutrient solution regularly. Metal concentration in roots and in soil can be measured 14 days after addition of metals. A metal accumulation potential is calculated by dividing metal concentration in root tissue on a dry weight basis to metal concentration in soil, on a dry weight basis.

Transformed plants demonstrating higher levels of iron accumulation than parental WT plants are selected for further propagation and, possibly, breeding programs and production using methods well known to those skilled in the art.

It must be noted that as used in this specification and the appended claims, the singular forms “a,” “and,” and “the” include plural referents unless the contexts clearly dictates otherwise. Thus, for example, reference to “a metal” includes mixtures and large numbers of such metals and heavy metals, reference to “a transgenic plant” includes large numbers of transgenic plants and mixtures thereof, and reference to “the method” includes one or more methods or steps of the type described herein.

The publications discussed herein are provided solely for their disclosure prior to the filing date of the present application. Nothing herein is to be construed as an admission that the present invention is not entitled to antedate such publication by virtue of prior invention.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications cited herein are incorporated herein by reference for the purpose of disclosing and describing specific aspects of the invention for which the publication is cited.

What is claimed is:

1. An isolated nucleic acid molecule selected from the group consisting of: (a) an isolated nucleic acid molecule that encodes the amino acid sequence of SEQ ID NO: 2, 4, 6, 8, 10, 12, 14, 16 or 18; (b) an isolated nucleic acid molecule that encodes a fragment of at least 6 amino acids of SEQ ID NO: 2, 4, 6, 8, 10, 12, 14, 16 or 18; (c) an isolated nucleic acid molecule which hybridizes to the complement of a nucleic acid molecule comprising SEQ ID NO: 1, 3, 5, 7, 9, 11, 13, 15 or 17; (d) an isolated nucleic acid molecule which hybridizes to the complement of a nucleic acid molecule that encodes the amino acid sequence of SEQ ID NO: 2, 4, 6, 8, 10, 12, 14, 16 or 18 and which encodes a YS1 or an Arabidopsis YSL protein; and (e) an isolated nucleic acid molecule that encodes a protein that exhibits at least about 79% amino acid sequence identity to SEQ ID NO: 2, 4, 6, 8, 10, 12, 14, 16 or 18.

2. The isolated nucleic acid molecule of claim 1, wherein said nucleic acid molecule is operably linked to one or more expression control elements.

3. A vector comprising an isolated nucleic acid molecule of claim 1.

4. A host cell transformed to contain the nucleic acid molecule of claim 1.

5. A host cell comprising the vector of claim 3.

6. The host cell of claim 5, wherein said host is selected from the group consisting of prokaryotic host cells and eukaryotic host cells.

7. A method for producing a polypeptide comprising culturing a host cell transformed with the nucleic acid molecule of claim 1 under conditions in which the protein encoded by said nucleic acid molecule is expressed.

8. The method of claim 7, wherein said host cell is selected from the group consisting of prokaryotic host cells and eukaryotic host cells.

9. An isolated polypeptide produced by the method of claim 7.

10. An isolated polypeptide or protein selected from the group consisting of: (a) an isolated polypeptide comprising the amino acid sequence of SEQ ID NO: 2, 4, 6, 8, 10, 12, 14, 16 or 18; (b) an isolated polypeptide comprising a fragment of at least 6 amino acids of SEQ ID NO: 2, 4, 6, 8, 10, 12, 14, 16 or 18; (c) an isolated polypeptide comprising conservative amino acid substitutions of SEQ ID NO: 2, 4, 6, 8, 10, 12, 14, 16 or 18; (d) naturally occurring amino acid sequence variants of SEQ ID NO: 2; and (e) an isolated polypeptide exhibiting at least about 79% amino acid sequence identity with SEQ ID NO: 2, 4, 6, 8, 10, 12, 14, 16 or 18.

11. A transgenic plant which comprises at least one nucleic acid of claim 1.

12. The transgenic plant of claim 11 which is also a hyperaccumulator.

13. The transgenic plant of claim 12, wherein the transgenic plant has been engineered to express a hyperaccumulator phenotype.

14. The transgenic plant of claim 13, wherein the hyperaccumulation of metal is compartmentalized into specific plant tissues.

15. The transgenic plant of claim 14, wherein said specific plant tissues are edible.

16. A transgenic plant which has been engineered to express the protein of claim 10.

17. The transgenic plant of claim 16 which is also a hyperaccumulator.

18. The transgenic plant of claim 17, wherein the transgenic plant has been engineered to express a hyperaccumulator phenotype.

19. The transgenic plant of claim 18, wherein the hyperaccumulation of metal is compartmentalized into specific plant tissues.

20. The transgenic plant of claim 19, wherein said specific plant tissues are edible.

21. A method of removing a metal from a soil environment comprising:
identifying a soil environment containing a metal selected from the group consisting of antimony, arsenic, barium, beryllium, cadmium, chromium, cobalt, copper, gold, iron, lead, manganese, molybdenum, nickel, palladium, selenium, silver, strontium, tin, uranium, vanadium, and zinc;
planting at least one plant of claims 11-20 in said soil environment;
maintaining said plant in said soil environment under conditions and for a time sufficient for said plant to remove said metal from said soil environment.

22. The method of claim 21 wherein the concentration of accumulated metal in said plant is higher than the concentration of said metal in said environment.

23. The method of claim 21, wherein the metal is selected from the group consisting of iron, chromium, manganese, selenium, and copper.

24. The method of claim 21, wherein the plant is a plant of a species selected from the group consisting of Amaranthus, Brassica, Raphanus and Sinapis.

25. The method of claim 24, wherein the plant is selected from the group consisting of Amaranthus paniculata, Raphanus sativus and Thlaspi caerulescens.

26. The method of claim 21, wherein the plant is selected from the group consisting of Brassica juncea, B. carinata, B. oleracea, B. nigra, B. campestris, B. napus and B. tournifortii.

27. The method of claim 21, wherein the plant is selected from the group consisting of Sinapis alba, S. arvensis, S. flexuosa and S. pubescens.

28. A transgenic plant which comprises at least one nucleic acid of claim 2.


SEQUENCE LISTING (20 sequences)

SEQ ID NO: 1 - DNA, 2494 bp, Zea mays
  CDS (184)..(2232)
cttaagggtttggactaatgattgtaattgagattcttagataacgagttcgttgttctaagtgagatggcatgtgtagttgacatttgaaggatttctctatataaatcccggctactattaacatctcaacctcaccatcttctcttgatcgaggcagggatcaagcgaccgatacccgccatggaccttgcacggagaggcggtgccgcaggcgcggacgacgagggggagatcgagaggcacgagccggcgcccgaggacatggagtccgaccccgcagcggcgcgcgagaaggagctggagctggagcgggtgcagtcgtggcgggagcaggtgactctgcgcggcgtggtggcggcgctgctgatcggcttcatgtacagcgtgatcgtgatgaagatcgcgctcaccacggggctggtgcccacgctgaacgtctccgcggcgctgatggcgttcctggcgctccgcgggtggacgcgcgtgctggagcgcctcggcgtggcgcaccgccccttcacgcgccaggagaactgcgtcatcgagacctgcgccgtcgcgtgctacaccatcgcgttcggcggtgggttcggctccacgctgctgggcctggacaagaagacgtacgagctggccggggcctcgccggccaacgttccgggcagctacaaggaccctgggttcggctggatggccggattcgtcgcggcgatcagcttcgccggcctcctaagcctgatccccctcagaaaggttctggtcattgactacaagctaacttacccaagcgggactgcgaccgctgttctcataaacgggttccacaccaagcaaggagacaagaacgcaaggatgcaagtccgagggttcctcaagtactttgggctcagcttcgtgtggagctttttccagtggttctacacaggcggtgaagtttgcggctttgttcagtttcctacgttcggtctgaaggcctggaagcagacgttcttctttgattttagcctcacgtacgttggtgcggggatgatctgttcgcacctcgtgaacatctccaccctccttggtgccatcctgtcatgggggatactgtggccactcatcagcaagcagaaaggggagtggtaccctgcgaacatacctgagagtagcatgaaaagcttatacggttacaaggccttcctctgcatagctctgatcatgggagacggtacataccacttctttaaagtcttcggtgtcactgttaagagtctgcatcaacggctgagccgcaaacgtgctaccaacagagtggcaaacggtggagacgaaatggccgcgcttgacgacctacagcgtgacgagatcttcagcgacgggtctttccccgcctgggcagcttacgccgggtacgcggcgctgaccgtcgtctcagcggtcatcatcccgcacatgttccggcaggtcaagtggtactacgtgatcgtggcctacgtcctcgcccctctcctcggcttcgccaactcctacggcacggggctcaccgacatcaacatggcctacaactacggcaagatcgcgctcttcatcttcgcggcctgggccggcagggacaacggcgtcatcgcgggcctcgccggcggcaccctggtgaagcagctggtgatggcgtccgcggacctgatgcacgacttcaagacgggccacctgaccatgacgtcgcccaggtccctgctcgtggcgcagttcatcgggacggccatgggctgcgtcgtcgcgcccctcacgttcctgctcttctacaacgcgttcgacatcgggaaccccaccgggtactggaaggcgccgtacggcctcatctaccgcaacatggcgatcctcggcgtggagggcttctccgtgctgcccaggcactgcctcgcgctctccgctgggttcttcgccttcgccttcgtcttcagcgtcgcccgggacgtcctgccgcggaagtacgccaggttcgtgcccctgcccatggccatggccgtgccgttcctcgtgggcgggagcttcgcgatcgatatgtgcgtcgggagcctggccgtctttgtctgggagaaggtgaacaggaaggaggccgtgttcatggtgcctgcggttgcgtccggtttgatctgtggagacggcatatggaccttcccgtcttccattctcgctctggccaagatcaagccaccgatttgcatgaagttcactcctggaagctagaaacagtatggacagttcacggaaatggagaatgcgagatacactgaaggcatcaactgtggttggttgaagccatatgtcccctgtattatccttgttgtgcatgtgtgggttctccaagggctacagcactcgagattttttatagagtttatatgttttgttgtcatgttgggttcaacctatgtttatgttttgggtggaaaaaaagattacaactgtacatttttaacagaaattaatgctcattttggttgtgtat

SEQ ID NO: 2 - protein, 682 aa, Zea mays
MDLARRGGAAGADDEGEIERHEPAPEDMESDPAAAREKELELERVQSWREQVTLRGVVAALLIGFMYSVIVMKIALTTGLVPTLNVSAALMAFLALRGWTRVLERLGVAHRPFTRQENCVIETCAVACYTIAFGGGFGSTLLGLDKKTYELAGASPANVPGSYKDPGFGWMAGFVAAISFAGLLSLIPLRKVLVIDYKLTYPSGTATAVLINGFHTKQGDKNARMQVRGFLKYFGLSFVWSFFQWFYTGGEVCGFVQFPTFGLKAWKQTFFFDFSLTYVGAGMICSHLVNISTLLGAILSWGILWPLISKQKGEWYPANIPESSMKSLYGYKAFLCIALIMGDGTYHFFKVFGVTVKSLHQRLSRKRATNRVANGGDEMAALDDLQRDEIFSDGSFPAWAAYAGYAALTVVSAVIIPHMFRQVKWYYVIVAYVLAPLLGFANSYGTGLTDINMAYNYGKIALFIFAAWAGRDNGVIAGLAGGTLVKQLVMASADLMHDFKTGHLTMTSPRSLLVAQFIGTAMGCVVAPLTFLLFYNAFDIGNPTGYWKAPYGLIYRNMAILGVEGFSVLPRHCLALSAGFFAFAFVFSVARDVLPRKYARFVPLPMAMAVPFLVGGSFAIDMCVGSLAVFVWEKVNRKEAVFMVPAVASGLICGDGIWTFPSSILALAKIKPPICMKFTPGS

SEQ ID NO: 3 - DNA, 2196 bp, Arabidopsis sp.
  CDS (10)..(2031)
tcagtctccatggaaatagagcaaagaaggatcatgaagagagaaggagaagaagaagaagacaacaatcaactttcactgcaagaagaagaaccagatacagaggaagagatgtctgggaggacaatcgaaccgtggacgaagcagataacggtgagaggagtgttcgtgagcatagtgatcggagttgtgttcagtgtgattgctcagaagctaaatctcgcgacaggaattgttccaaatctcaacagctctgcagctttacttgcttttgtctttgtccagacttggactaagattctcaagaaatcaggatttgttgcgaaaccattcacaagacaagagaacacaatgattcagacatctgctgttgcttgttacggcatcgctgtcggaggtgggtttgcttcatatcttctggggttaaaccataagacatatgtgttgtctggtgtgaacttggaaggtaactctccaaagagtgtgaaagaacctggccttggttggatgactgcttatctctttgttgtctgtttcatcggtctttttgtcctaatccctctccgaaaggttatgattgttgaccttaaattaacatatccgagtggtttagctactgcggttctcatcaatggcttccacacacaaggagatgcacaggccaagaaacaagtgcgtggtttcatgaaatacttctcatttagtttcttgtggggtttcttccagtggtttttctctggtattgaagattgtggctttgctcaattcccaacctttggtttgaaagcttggaaacaaacgttcttctttgatttcagcatgacatttgtgggagcaggaatgatttgttcacatttggttaacctttctttgcttttaggagctatcctctcttatggcttaatgtggcctcttcttgataaacttaagggctcttggttccctgataatctcgacgaacacaacatgaagagcatttacggctacaaagtcttcttatccgtagctctaatcctcggcgacggtctttacactttcgttaagatcctctttgtgaccattgccaatgtcaacgcaagattgaagaacaaacctaatgatctagatgacgtaggtcacaagaaacaacggaaagatctcaaggaagatgagaatttcctcagagataaaatcccaatgtggttcgcagtttccggatatcttacattcgctgcggtctcaaccgtcgtggttcctctgatatttcctcagctcaaatggtattacgttattgtagcttacattttcgcgccttctctggccttctgtaacgcttatggagctggacttacagacattaacatggcttataactacggcaaaattggtctttttgtcatcgcggctgtgacgggaagagagaacggagttgtagccggactcgccggttgtggactgatcaaatccgttgtttcggtttcttgtattttgatgcaagatttcaagacggctcattacacgatgacgtcacctaaggctatgtttgctagccaaatgattggaacggtcgttggatgcatcgtgacgccgttaagtttctttttgttctacaaagcgttcgacattggaaaccctaacggagaattcaaggctccttacgctttgatttacggaaatatggcgattcttggggtgcaaggcttctctgctctgcctcttcactgtctccaaatgtgttacgggtttttcgggtttgctgttttggtcaacgtcgtcagagatcttactccggcgaagattggaagattcatgccacttccgacggcgatggctgttccgtttcttgtcggagcttatttcgcaatcgacatgtgtgttgggactttgattgtgtttgtttgggagaagatgaatcggaagaaagcagagtttatggttccggcggtggcttcagggctaatctgtggcgaagggctttggactttacctgcggcggtgcttgccctcgccggagtaaaacctccgatatgtatgaagttcttagcttcataggatcagtcaaagagataagaagacttgtgaagttagagtacttagaagacgtcaatgatattaaaatcatcctattcttagaataacaggtagacttctgaacaagaacatctcttcttctaggaattaagactaaaatctacttacatccggatgattcatgct

SEQ ID NO: 4 - protein, 673 aa, Arabidopsis sp.
MEIEQRRIMKREGEEEEDNNQLSLQEEEPDTEEEMSGRTIEPWTKQITVRGVFVSIVIGVVFSVIAQKLNLATGIVPNLNSSAALLAFVFVQTWTKILKKSGFVAKPFTRQENTMIQTSAVACYGIAVGGGFASYLLGLNHKTYVLSGVNLEGNSPKSVKEPGLGWMTAYLFVVCFIGLFVLIPLRKVMIVDLKLTYPSGLATAVLINGFHTQGDAQAKKQVRGFMKYFSFSFLWGFFQWFFSGIEDCGFAQFPTFGLKAWKQTFFFDFSMTFVGAGMICSHLVNLSLLLGAILSYGLMWPLLDKLKGSWFPDNLDEHNMKSIYGYKVFLSVALILGDGLYTFVKILFVTIANVNARLKNKPNDLDDVGHKKQRKDLKEDENFLRDKIPMWFAVSGYLTFAAVSTVVVPLIFPQLKWYYVIVAYIFAPSLAFCNAYGAGLTDINMAYNYGKIGLFVIAAVTGRENGVVAGLAGCGLIKSVVSVSCILMQDFKTAHYTMTSPKAMFASQMIGTVVGCIVTPLSFFLFYKAFDIGNPNGEFKAPYALIYGNMAILGVQGFSALPLHCLQMCYGFFGFAVLVNVVRDLTPAKIGRFMPLPTAMAVPFLVGAYFAIDMCVGTLIVFVWEKMNRKKAEFMVPAVASGLICGEGLWTLPAAVLALAGVKPPICMKFLAS

SEQ ID NO: 5 - DNA, 2316 bp, Arabidopsis sp.
  CDS (156)..(2150)
cctcgagtctgcgaatcattcgattctcagattcgcatattttcttcgaattcgctaggctcaaaacgttgtttccgtgtacaagaagcaacctaacgatttggggccaggtgaaggaagagctgatttaggttattgagattcgttttcttcaaatggaaaacgaaagggttgagagagaacagagccaatttcaggaagacgagtttatcgattcgagaaaaccaccgccatggaggaaacagatcacggttcgagcgatcgtcgcgagtttattgatagggattgtctacagtgtgatctgtctgaagctgaatctaacgacaggactcgtaccgaatctcaacatctcatcagctctcttggcttttgttttcctcaaatcatggaccaaagttcttcaaaaagccggaatcgccacgacgcctttcacacgtcaagagaacactattgctcaaacttgtgctgttgcttgttacagcatctctcttgcaggtggatttgcatcgtatttgttgggtttgaatagacgaacttacgaggagacaggagtgaacaccgaaggaaacaatcctcgtggtataaaagagcccggtgttggatggatgacatcattcctcttcgttactagcttcattggacttgttgtattggtacctcttcgaaaggtgatgattatagactacaagctaacttatcctagtggaacggcaacagctgttcttataaatggatttcatactagcaaaggagataagacagccaagaaacagattcgcgggtttataaaatcgtttggtttgagtttcttttgggcattctttggatggttctattcaggtggtgaaaaatgcggattctctcagttccctacatttgggttacaagctttggacaaaacattctactttgacttcagtatgacatatgttggagctgggatgatttgttctcatttggtgaatctgtcgttgctcttcggtgccatcctctcgtggggaatcatgtggcctctcatagcccggcttaaaggcgaatggttccctgcaacattgaaagataatagtatgcagggcttaaacggctacaaggtgtttatatgcattgcactgatcttaggagatgggctttataacttcgttaagatactctttttcactggaagaagcttccactctagactctccaaaaccaacagcatcagcacattggtagaagttccagaagatagcacgaaagaatccgataacctgaaacgagagaatgaagtgttcgttcgagagagcattccgctatggatggcttgtgtcggatacttattcttctcccttgtctccatcattgccatccctctgatgttccctcagctgaaatggtacttcgtcctcgtagcttacctcctcgccccgtctctcagcttctgtaacgcctatggagctggcttaacggatatgaacatggcttacaactatggtaaagcggcgctctttgtgatggcggcactggcaggaaaaaacgatggagtggtggctgggatggttgcttgtggtctcatcaaatcaattgtctctgtctcagctgacctgatgcatgatttcaagacgggacatcttacgcaaacctcgccccgctcaatgcttgttgcacaagccatagggacagcgataggctgcgtggttgcgccactcaccttcttcctcttctacaaggcgtttgatgttggaaaccaaaatggtgaatacaaagctccttatgctatgatatacagaaacatggctattatcggtgttcaaggtccctccgctctccccaagcattgcctagagctttgctacggattctttgcctttgcggttgctgccaacttggcaagagacctcttaccagataagccagggaagtggatcccactcccgatggcaatggctgtaccgttcctggtgggtgggtcgttcgcaatcgatatgtgtatcgggagcttagtggtatacgtttggaaaaaggtgaatcgtaagaaggcagatgtgatggttccagctgttgcatcaggtttgatatgtggagatggtctctggattctgccttcttctttgctggctctggctaaggttagacctcctatctgtatgaacttcactgcggctcattaaggcagaaagattttccagacaaaacccgaacttaccgccggagaaatgtatagtagatgtggttaggttaggagtagacagttaaagagggatgaaaccaaacttacatgtcttatgtaatatttatacttttggaataagaatgaacagaaagagagttggtttc

SEQ ID NO: 6 - protein, 664 aa, Arabidopsis sp.
MENERVEREQSQFQEDEFIDSRKPPPWRKQITVRAIVASLLIGIVYSVICLKLNLTTGLVPNLNISSALLAFVFLKSWTKVLQKAGIATTPFTRQENTIAQTCAVACYSISLAGGFASYLLGLNRRTYEETGVNTEGNNPRGIKEPGVGWMTSFLFVTSFIGLVVLVPLRKVMIIDYKLTYPSGTATAVLINGFHTSKGDKTAKKQIRGFIKSFGLSFFWAFFGWFYSGGEKCGFSQFPTFGLQALDKTFYFDFSMTYVGAGMICSHLVNLSLLFGAILSWGIMWPLIARLKGEWFPATLKDNSMQGLNGYKVFICIALILGDGLYNFVKILFFTGRSFHSRLSKTNSISTLVEVPEDSTKESDNLKRENEVFVRESIPLWMACVGYLFFSLVSIIAIPLMFPQLKWYFVLVAYLLAPSLSFCNAYGAGLTDMNMAYNYGKAALFVMAALAGKNDGVVAGMVACGLIKSIVSVSADLMHDFKTGHLTQTSPRSMLVAQAIGTAIGCVVAPLTFFLFYKAFDVGNQNGEYKAPYAMIYRNMAIIGVQGPSALPKHCLELCYGFFAFAVAANLARDLLPDKPGKWIPLPMAMAVPFLVGGSFAIDMCIGSLVVYVWKKVNRKKADVMVPAVASGLICGDGLWILPSSLLALAKVRPPICMNFTAAH

SEQ ID NO: 7 - DNA, 82952 bp, Zea mays
gatctgttgttctggctgttatagttgaatctgacagtgtttttaagcgtcaatggctcgtgctttgatctcttattgctattacatggttttttcgattcatagtgatttagtagtctttcacctttatggctttttggatcgttgacgagtttcttcatgttgttggcttaatttactgattctgtcttacttcttctttattttcagttttgctgaatctgtttgtttctgttgcagataaacctatggcaatggaggttacgcagttgctcattaatgctcagtcgattgatggaactgtacgtaagcacgcggaagaaagtcttaagcagtttcaggagcaaaaccttgcgggtttcttgttgtcgcttgctggagagcttgcgaatgatgagaagccagtagatagcaggaaattagctggtttagtccttaaaaatgctcttgatgctaaggaacaacacaggaagtatgagcttgttcagagatggttggctctagacatgtcaacaaagtcgcagatcagagctttcttgttgaagacactgtcggcacctgtgcctgatgttcgttcgactgcatctcaggtcattgctaaggttgcaggtattgagttaccacagaagcagtggcctgagctcatagtgtctcttctctcaaatattcaccagttacctgctcatgtcaagcaagccactttggagactcttggatacctatgtgaagaagtgtcacctgatgttgttgaacaggagcatgtaaataagatactcacagctgttgttcagggtatgaatgctgctgaaggtaatactgatgttagacttgctgcaacccgtgctttatacatggctctcggattcgcccaagcaaatttcaacaacgacatggagcgtgattatatcatgagagttgtgtgtgaagcaaccctgtcccctgaggtgaaaattaggcaggcagcttttgagtgtttggtatctatcgcttccacatactatgagaagttggcgcattacatgcaagatatattcaacatcacagctaaggctgtaagagaagatgacgagtctgttgctctacaggcaattgagttctggagttctatttgtgacgaagagattgacatcttagaagaatatggtggtgagttcgctggggattctgatgttccatgcttttattttactaagcaggctctccctggtcttgtgcctctactgctggagactcttctgaagcaagaagaagatcaagatttggatgaaggggcttggaatattgcaatggcaggtgggacatgcctcggtttggttgctagggcagttggagatgacattgttccacatgtcatgccgtttattgaagagaaaatatcaaagcctgattggagagagcgagaagctgcaacttatgcttttggttccattttggaaggcccttctgccgataagttgatggcaattgttaacgcagcgttaacatttatgctcaatgctctaacaaatgacccaagcaaccatgtgaaagacacaaccgcatggacccttggtcggatatttgagttccttcatggttcaacaatcgagacacctattattaatcaggcaaactgccagcagataatcacagtactcatccagagcatgaatgatgcgcctaatgttgccgagaaggcttgtggggctctgtacttccttgctcaaggctatgaggatatcggtcccagttctccgttaacacccttcttccaggaaattattaagtcacttttagctgttgcacacagagaggatgcaactgaatcacgcttgcggactgcagcatatgaggcattgaatgaagttgtcaggtgttcaactgatgaaacgtctaccatggttctgcaattagtaccagtgataatgatggagcttcacaatactttggaaggggaaaagctttcattggatgagagggagaaacaaaacgagttgcagggacttctatgtggatgcttgcaggtcatcatacagaaattaggatctgaaccaaccaagtctaagttcatggagtatgcagaccaaatgatgggacttttcctgagggtgtttggttgtagaagtgcaactgcacatgaggaagccatgcttgccattggtgctcttgcttatgcagcaggtcccaattttgccaaatacatgcctgagttttataagtacttggagatgggtcttcaaaactttgaagaataccaagtgtgtgctgttactgttggtgttgttggggatgtctgcagagcattggaggacaagattttaccttattgcgatgggattatgacacagcttctgaaagatttgtcgagcaaccaattgcaccgatcagtgaagccaccgatattctcctgttttggtgacatagcacttgctatcggtgaggattttgataagtactggaggtattcaatgccgatgcttcagagtgccgcagagttatctgctcactcggctggagctgatgatgaaatgacggagtacacaaactcattgagaaacggaatccttgaggcttattcaggcatatttcaaggtttcaagaactccgccaaaacccagctcctgattcctttcgcaccccatatcctccagtttttggacagtatatacatggagaaagacatgtaagtttggttacgacccaactgaatttctattatgtcatagcagttgtttaattttgtggtaaaattgccttgggacagggacgaggtggtgatgaagacagcgattggggtcttaggagatttagcagatacactagggagtcatgtgggtggtttgatacaacagtcagtgtcaagtaaagagttcttaaacgaatgtttgtcttctgaagaccacacaattaaagaagcagccgaatgggcgaagcatgccataacccgtgccatatctgtttgaaggctcttcttttgcctataagaaagaaaattccttttgctttttagtaaagtctgcatgcatctctgggtgggttgagtcggtcggggggaggggtagagctgcaacatccgagtcacatcagcatcatcagcatcagtttcatttgcatcatcagcataactggggtcagtcagggaatcgttgagtcgtggatgtggggaatgatgagttttggggtaaataatgaaactggaagagtcacgggtgaaagtttcccgggtaaggttaaatccccgggagtagaaagaggaggaagtgagtcatggtcgttttctggtgggttttgggacaaagttataccaaaaaaaaaaaggtttcatggaacatctcataatacattcttgttttttcttttctctcatatgttcttcaaccccttgaggagggagaggtctcaggtttttatcaagtgggggcattattagaggacaatcccttgattttttcttttgttttcttttattatttggttagttgcagtctttgcatcatcattttgtcttttgggggggtttgtatgtttgattaaaagcattattcacatttttatttttatataaagaagtttcacatgtctgtctatcttctctttgctatgatgcagtttagggggttttgcatatatacttgtgttttgtttgcaatcaagtttcaaatgtcacatacaggaacatttttattatatggtgtcatgtcttagggtcttaaagaggtccacatctattcattttactatggaactgtttacaatcgtttagaatccattatttgatttggctcgaactatatgggaaagggcacatgttaataggagaaaacgtcctgccttactctgcttccaaagagtcaagttaagagatcgaagaacatttcacattcacgctgtttatggaaacctcaattttgattgtatgtgttgcttttttttggttttccaaacgccaaatgcaactgataccgaacctatattttgaaaaatgttttcatgtgtatttgaaacattctagaccattcaagttatggtttaaattagtttctctcggttctatgtattagtcccaaaccatgtttctgacgtctcgagaatttttctcttgaactaaatatataatgtcataattcattcgacatatctttttcgaattgatgatttaggcgagagaaacgttggttctgttggtatttttttttttttcattgtgatgacatacccatttaaatctcactcaaatggacaataaaacacataaaagatagatgatggctaaacatccaagagaaataggacacgccactctgaattgaaattgggagttgaaaaagaatggtggtaatacgaagtgggccttttttattgtcaaaggacttgtccaagcacacccacatatatcacatacactgatacacacacaaaaaaaaaaaacatttttgtcgtccgcgacgatttttttcatattcaaataatttcattacggcgttaatactttattttgcgactttaaactttacctttagtctttaaatgcaatactgaaatagcaccgaaagatatatgagcgtcgtcagctgaaactgccaaaaaaaggagaaaatcttacgatccaagttattagtcaaaagaaacataattattgattaaagcatgaacttcttgcgacaaaaaataactccaatttttctttcaatagctattatctaacacattttaccttaaggttacggatcaatacatttttaaaaataataatgattagtaaatactttcttagaagatgaacgccattaaagaaaactctgaaaaaaaaaaaaaaacggatgttttttaagaaaattaattcgtttagtaggacaacgaaaacctaatatataagctaatttaatggataaaaactagaactttggttcaaactacatttatagatctaactatgcgttctcattgcctgatggctttcattttttgatgttgttccttctaatgttgatacgctcttaagcgatgatgttattgggtatgtgcgcctaaggcaagtaagtttgtaatttttcttataatttttagtctcccgtcttctacagaaaaaaaaactacacttatgagatgaattgtatattaaaaatttaaaacgaaggaaatatttaagaaaagtagtgaaattattagttgtgggtgtctcgtgatttattattcgcgttgtaaactatgttagtattgaaattgtgtttttgttctcttttttttttaactaatagacgtacgttttttttttgtcaaagactaatagatgtacgtttcgtcgtctttctgtgtagttgacattggacaacagaagttaagacttaagacacgtacgttgttgcccaataaaacattgctaatttagtggctcattggtttcttattcatcactccttttaaacgcaacatataattgccgacacattgattatttccctactcacgaactttcccttttttttttttttttttgtgaagtcatgcatagtcttcggacagaagagctgcacgtacatgtgttaactgttaagtgaataatgttttagatagtacaacgattctacttaagcagtttagctggtttaatgtacgtgtagtctatattagtttttatcatattcagatgctgaaaaagaatctgtttcattttattttttatagaaaactgcagatatttacttggaggttgtgagaaaacattttaagatggaaataattaaccagttaagaaagttgaagtgcgaaaccatttttttatatgaaaggatctatagaggggatttataaagtcttctaatctaaaatatatgggttatggtgcaatacacacacacatatatataagtataaccattcatatggtgactttttgggggtaaaagttctttagtttttgggatttagtagaattatggattttaggtcaaaacacataatgttatgaggaacgatgtttattaattgtaaaataagattttgggggatagtggtctctcactataataatgggtcacttaaaaagatgcacttctctgatacattttgcaggccactcgctatgtctgtctcaatcctagttgacttcttataaagttgtgaaggccgaaatggaaggattagttccactatttctctttatttgtatcaaattatttagtgtttggtaccacggattttaaaatctgccctaaccatcttttgacttcctaattacttacgttattcccctgtctaggagttgcctaccatgttggtttatgtgatcgaagtttactgaacttagtctatctattttgatccctgtaaaatttacattgaccgcaaggaaaatgttttgattccacatttaaactttaatttactagtactgatctttgttctaatatgtcaactgtacatggtgtatttaaatataacaaccaaaatttcaaacagaaattggattaaaaggttttgaaaataatcattggattaataagtgtgttcaagtttgggtatttgtagggtctttgtttgaaaagtgtgtacccttgatacaaatcttcgctttcgttttgattcattaacagcattgaaaacttacaataattacgtacgagtttggtatgattccatctttacttttgataatggacccaattatcttagcagaattcaatttccaataaaattttgcgtcaataagtagacttaagatcacacgtggtgctgcactcatcgaacctatgatacattacagctgtatgggaaaaagtggtgaaaacaatgggcttaaaagttaaaacttagattaaaatcgatcatgtaaccagaatgaaacttttcctttaagaaaatatattggttttgactaaaatcatctaaaatgaaacatcacttagaaataagaactagtataatagagagtgttttgagatcaaaccgattaaaattgtaaaataaaaaaaaaatgttttctttttttaatgaaactcaaaggatataatgaaaatttatatggtcaagagatgaacttccactcaagttaatgaattaggtcacttttcttgcatcaaacaaaccttttttcattatgatttgatccacatatataacctttcggatggcttagggcacaacttactctaattcaagtatttgatggttcccacttcacttggttcacaaacatttacagttagtaatctatactcagatttaacttgttattcctaattacattttcctatctggcaaattaacaatatagataaactatttatctatcatcaaaaattgattgccagaagaaaaaaaggctaattaaaagaaacgtcaactttttgtttggtagttaaagttttgtgacttttgtctatagtatatctagtacggtctaattaacataatcataagataaaaacgatatacagaatttgaaatttgaacaatgtcatgctataaggaaaagtgtgttaagaaaaaaagtgaggttactagctaatatacgatttttgtccacaagcataaatagaggtaagcgttaagtgttcctttttgggagatgtaacacaatcaacagttaagttgactttttcaaccttttctagggtttgtggcctgtaggttcattgcgttaacccaataacttcttattcttctcatttttgttcttgctactatcatcaccattttgatgttagtagtctcaccactgttttcacctttttttgataattgttttgtgatatataaaaaaaatcgatatctacgtgatattcttattttaaaaagttatattgataagagctttacatattttcattgtcataaacagaaccaaccatcttagtcatggtcatcatcattgtcatagtcatcatcatcaccattacgactaatattatacatggaccataacagtttccaccacattaacttgaccgtttataaaatgggaactaaccttccccatcttctcatatgaaaatccattttgtcctcattttccattgattagttgcattctctcccctaaacgaacaaagagtaatcatcaaaaaccatataatatgcctcttaggattgaacccctatctctactcttcctcattctcattatatgctcttcatcattaggtaaacatataattgatattcatatattccatccttttcaatttatagtcttttaactttgttttaagggatgactatcaaccttgcgtcccaacacatccatcttagtccgagatcagcttttggtattatcttgttgaacattagtcttttcttcgttgttgaatctagtgccttaaactaccttaataccattgattattaattactattatattggtgaacactttttccattctttctccaaaattaaaacacatctaactaactagtatgggttgaaataaataagtcacttaaagaacaatgtatattccgctaacagagtgttttcttttagccatagtggaaggctctatccagctcgacggtgcgaagatgcatgagagttccatgctttacaaggtatacatcatacatgtagatgctttacgtttatatatgttcttatcaacattagaaatgttgttaatgtccaaatattgacaagaatttatgtgtttataattaaaaagagtgaggaggtgggtgtgaagaagatgggtcataggaagttgatgttccactcgacagcggactacgacgacgcaggacccaaccctaaacacgacccaaggagaaggcccggaggcaagggttgaatcatcaacacagaactatctctgtcattatatatatagtagcatatatataagcacatgcactaatatctatatatcatatatgaatatgtatgtgatgtatcctccattctgactctttttgtttgaggatgagctcagtgtaaaatacccttcggaaacccgctagtgaaaggattagtgtctaataacaagacataataaatccttatttattatcatcttatttcattgacatatataatatagccattatatcctgtgtaatcgttatatcagaaaataaataaagtgttatggttatttttagttctgtatagtattattataaatgcgtcttctacaatatttcacattagttgatgatttcaatcttatgcagtcaaacacatagtaaaaaactctttatcagcatgatcactaaacgaaaaagaaatgaagacagattttatgcttatcatagctaaaaccttatatgtcgctattacaataacatagtacaaatgaaacaagattgcaatggttgatttaaaaaaagaaaatataaaactaaaatgaaaaaacaaatataaaattaaaacagttgcgcatccagggaatcgaaccctggtcagtaccgtgggagggtactatgataccactacaccagatgcgcttttattcattgtctcttctcttacatatttatagcgaagactagttactatggttatacatatttcacaagcatacataaaattcacatactaacacttaaatatgtctatgtaaattattgaagagaaacagagaaaacgccttttgtttggttacagaatttacaagaatcgattatacaacctctcataactctcttgactcctatccttcaacctttacttagtttcatacatctttgtgtcaacaaatcttaaatcttacatatataatacattcaaatcaaaaccttctctcttctctatcggcatcccaacacaagtcttccttcttcgtttatggactcattcctacatattttctgaaggtctatgtaaccaatgatagtgtcctcaaagaccacatcctccaaatttgcctcctcaaatgtcgaaccggataataccgtgttcctaaacaccgcacctttcagattcgatttcccgaaattcacccgatcgataacagcattagtgaaattcacccctagaaaagaattcacactaaaacattaattaacattttggtccataattttaccatatgctttcaaatttcaagatttaagtctatatacctttaaagcttgcttctacagcataagcttttgacataacaacttcagtcatatctgcaccatcaaacttagccccaaccatcaaggctgcagagagagtcttgcctttgagattagtttgatcatttgtgtaatcacagaacctaagatccagtggtttgtcatagacaccatttgcttgacctatcgtgttaccaacaaatgctttttcacaccggtctggttctgttgataacggaggaagtctctgaaaaaaacaatccatttatccatttctttaaattctaaaacatagccatcttcagacacaaacatagaaaagagaaagggccatataatgtgacagacctggttagcagcaataacaggagaagctattgtaagagtgcaagcacagagagctgcgcaagcgatagatccaagctccttaagaggaaagagactcttctttacaccttcaccattttctctgttttctcctgctgagaacataatgcaaatcaagatcacatacactactttagcttcactttaaaagcaagattttgacataacaatattcataaacccatctataaagatgaattcattgaaggtaaagctaaagattcttgaaaatacttagaaatttcgaagaaccgtacaaccaagaagaagtagcagagagatcttgaaaaaccaattaaagcagagagaaaaaagatgacatttaccggagcagtgtactgttactaaagatctaggctcacggcggaggtttacagagaaaaacggtgaagagatttgattcctcgtgaattgaacaggaagcgaagccatggaagtgtgtttcagatttttgttcgaacttggacaaccatagagttttatcaattcgttatggatcatgcctatgacgtggcatgacagatcataatatcagactatatacgatttagcccattgggcctgattactatttggtccatttgtaaatctttcatcggttttggatataatatcaagtagtgcagcccatcggttaaacatctacagtatattattgatgacttctaacccacgcaaaatatatgatttaccatgctaaatacgtaattaatagcttaatagtaaatattttattttatatatttttatttatgttaaaataaaatttatatttgtatttattacattaaaaacgattttaaataataaatgattatatttattttgtgtaagctaataaaagatattaaaaaaattgattagtatgagaacatcttctacaaatgtaaggttatcttataagatacactcggaatattattagtaagatttttgtttccatagtctatcggatccttctaggtcaattaaaagttaaaaccctagagcacaaaaccatcattcatgattgcacacactaatttgtttgacacaacaatttcctaacattcaaccacaaactcttaccttatctcgatttggtcaatcagaaaattttcagattcattgatcatcatttactatgtgttctcatatatttgcgatttcttttactgcttagtcaaagagtgggtaaatcgccaagacaagtttcaacaattcttgtaattcatgttacaccacaaactctagttttttttaactgtatgtacttggaagaaaacaagttttactctgtacaaaatttacagctacaaatctccggagcctctctttgaggtggagagctcttacaatctattgatttgtctcttataataagtggaaatcaaatctagaacaaagctatataacaaactcatctacacgttagagactcaaattacacataatcaacgaatcaataatcactacgttagaaatttatcaatacaaaatcctaaattgaaaaaaggatcttaggagaaggcattggaagatctttcttgtggaagtaacgtcaacatctttctccttcaaccactcctttacatgatcactactttgcctctcttggttttggaccctacaataatcgcctgtcaattttcagacaaatccgtactgtctaaaaaccatctccaaccgccactaattgccaaaaacagaaacatctgttccttatcatctcctgcgtctacatcaaccgattgaaacctctcccactgaccacttcttcaaagatttttctacaagatgatgataaatcttgaggagaagtaacagaattgtatcactttttgactaattgatgtatctccagagatcttagagtggatctggttttggacccaccattgcttcatgatctgctagaaccatgtgcttgtgcctatcaagataggaacaatggcatctgatttgttttccaaagttgggaaaacgaaacaggttttttcaagtagtcacaggcaacccgtagttctgaaatgttcttatctgtccatcccaaccacctgtgactatgaccataccccaagatagagagctggatgctctctggtaagaggacttgagaaacttgtaatgagatgcattcatcgcggatgtggacagaggatttgcgggtaggttttcctctggccatgtcgcactccctttggatattgatccggggacaaatccttcgttgtgagataagcatgatggagaagaaaacgggagagttgtgttgtgatctgagaatccacaccatgttgctgcaactgatgcattggcggagaaacgttcaaaagatcgaatctttttggtttctgattcctcatcattgctccatatatatacattggagtcctcacaagctgaaacaatgtgttttccatcagatgtgagagatgctgatgtgaggcttctcgtcttgcaaacgcctaaagattgaaaaaacagagatgggattagtttagaatcaataatagagctatgcaaaagaaaatgaaagttgaaggagacataaaaagaaacatacctttgtattttctgacgacattgttgccttggagaattctgactttggaatccgcggaaacaaccaggactttagtcggttcttgtggtaagaactggaaaaagcaagtacgatttgttaacatctcacaaatttcaagagaagtaaacgaagtatctagagaaagggaaagacataggagacattacctgaaaaccagttatgcgtttatttgaagacttctttttgttatgcaaatgtatctgagagtccagttctaaaaattctccagacatgttaaagaatctgcaactcccgttcagagaaccgatgattccaccttgtccatccggacgataacacactgccgatataatgtctttgagatctgcccaatcaacaacgctgcaaccggaaatgttccaaattcgaacctttccatcaattgaaccactcatgaaataattctcatttaccgggttgaactgaacagaggttactgcaaaaagaaagttgcaggcaacaggtaatgtaagatcaatgtagtaactgaaaaagaaaaagagatctagaaaccaaaaagaagaagaagcataccataactattgtgagcgaaaactccaagacaatcattgctaccaactttccaaaggcggacagttttatccattgacgccgaaagcaaatactacaccaagaaattacataacaaaatgcctcagtttttcgcttccgttaaagcaatggttgcaagttaatataagaaaatgcaaaagattttggaggcttacattatcttttgaccatgagatgtccaagacttcacccgtgtgcccacggaattcatataatggtttttccataatccgaaaaactttaggagggaagacaacacaggctgaatcagatgttttcctgaaactttctgtagtcttctttggtttctcctcatttaccaaaactggtttcaactgagaaagatcatttacctcaaagtacatacacgaaggatctatctcgttaaggcaatcccttcgtagtctagatttcttatcctcaacgactttccacaccctcacaatcccatcttcaccagagcttgcaagaaacttcccatcgttgctaaatttcatcgccaagatggcaccatcatgagctttaatgtcttgactctgataaagagctgaaagctcctttgcctgcttcttacaatgcttaaccttaactcttgaaatcacatcaccataaccagaagaagctctcattctaccaccagattcgatatttgtatccgcggtacatcccatagaccgaagcctcgataaccatcccttcttaaaactcgtgactagccctgcgatattcctctgcttctcggtttccttcacttggcaatatctggaactagaacatgacgccattgaagagctttgtcttctcgtcacatcacatccacccacatttctgtctactctcaaagacaattcctcggaaacttcaacgcttgaaccagacaatgaaacctcactagaagaactgctgcactctccagatctctcctcagcttcaacagaattctcttcacctccatcaaaacaagactccacactacttccagaaaccgaatccttcgtctcaacacgcccttcttcttcaactcccatccactccaagaacttcctccgccgctccacaacacttccaggtgaatcattccatacatcaaatccaaaaccacttggattcaatgccatcatctcttcgttagcatcgaaaaactgcaaatcttcctcttgagataagtaatccattattccttgagctattgtcaatccatgaaacagagcttaaagaagaagaatgcttttctctcaaaagaggaatttccccaactgccagttttgcttccaaacaagcaacctacaaggcaaactatatataatactttagacaataatattttaaagaagtttacaaattaagcaatcacagagttcgagataaaaaaatcagtttaaaaaaagaaataaaaagagtttaacatcaatctcaatcctaaaacccattgaatctagtctctctgagacatttacacaaacaacttatgtgggtcgataaatgtgtaagcaacgaaaccaaaaacccatcaaaagagaagaaaaaatggtttatttagcacacagagaagctgaaatcagcacaacaaagtgaaagatctcatttttagatcctaagcaacaaaaaatccgcaacaaaaatgcgtgagcacgagcatcaccaattcaaattttttggtaacaaagattaaaaattgcgtacatgggttggttcgtcttcacgaggtttcttcgaaattggaacaaacacatcccaaagaagtagcaacagtggtagagagtctaaagaaacacacacaaaccaaaaccagagagaattgtaaaaaagtaaaaagagagaaacttttattattgaaagggaaaagagaagagaagaaggatcaaaggatttttattgaaatctgtgaagaaagagacaataaggaagaagaagaagatgggattgtccggagggaatagagagtttttggagacgaatctttgtgtgtgaccgtttgctttgccttatttaacctctctgtctttatttcaattttgttttcttctcttcagattattcggtaattaaatctattaatcacacgaccaaatatttcgtagtaattaattataatcatttgttatcaattttagatctttttttttatctcctcgtgctctctgtaatactactactaatcagtaataaatcattccctctgtatatgattaggagaattaatacatttatatgtgattctattgaaacatttttttaagttatttaaattttattttttatgaaatataatgatattattttagagttttattcattttcgttacattatagttttaagggtcaagaattattaatcgtttatgcgtagagtttctttttgatattttgaagaaaatattttgtggtacatgctttgttggagcccacatatttgcatgatgactacgcggcatgttccctattcgctttgcgtttgatcagattctaactatagtcctttgatctcaattatctgtcacattaacaatctaatttccagttgtttgataccttaattattcatcttctaataaactaaagttctttttaacttttttatttgcctcttagcactctgtgattgtttggcagcacacaaggaatatttgttactcttttaagtgatcatcatagagatttatattaaaatataggatatgatttttgggttgttccttatcaaattaaaaattgcaattaggaataatgatttaattttaatctataattaagagaatgatgtgatttatatgagttatctatatctaatcatttaaatggaccaaatacaaactaaaccgacaagattaaaacgaattgtactatatatcaagtttttatttatcaacataattgacgactaaaggccgtcattgctgagctaagtctgtctttctgttgaagcaattcacaagtagaaattaattaagcaaataattaacaatgtaatgatgaaatactactgcctttagagttgcaaggcagagtacctggattcatgcaggcttgtattgtaaagagaatatctgttttcccatttacatttttgtaattatttatttcgagtcttaaatttttgtttgttacttaaacaaaagatcttcaagatttcttgataataatgatgttagtatgcatgcatcatcatattaaaatagacactatgggcaacttggttttctcctcctactattttcatcttcatccgagaatctgagactctattagatataatctaaaactcttttttttctacaagcaccttaatgtattttattttatttttttttcgaacaagcaccttattgtttttatgttctaaaataaacgtaagaatctagactttgcctctcttttttgtgatatttgattttctctcctattagttacacaatcgttttttcaaataaaaatgcttcggaagaatttgtaagaaggtaaaacaaaaacaaaaactgagaaaccttttttgataaaagtttaatatctcacaatttaattagctgaaatatgagtctagttattatattatagatcatctagaatatgcgtacacatattattctatgatccatagtgtttattttgtataaggtaataacagcaacaacaacataatttaaccggttagttaataacattttcatatagctagttctctccatttatatatccgttgcgacaatttcaagtatgtagatattattagatttgttactaatcgaggaaaactatgagcttaattgcaacaattcgaatgtttagaacttatgagagatgttctgaaacgaaggaacatgttggcttattgacagccatcaaaaatttcatcatatcgtgcataaagttcaaatccaaaacttatctccttatctttttgtaagataaagtaaaagagttttgtgtccaagtaaaaaagaagtgttcgtgtccaagtaaaaagagaactattattgattttgtcagcaatgtgttttgtgtagcatctataatgtgctgatgtggcttgccagctaggtaagctatcaaaagcaacagtttaaattatagataaaaagcgcacgaccatccatattatattttatttttgcaggatttctatagtttatgacttatgatgttattatatttgcatattggtataacaaattataaaaatattgtttcaaaaaagatacggttcatagaatggccacttcattgctttggttgttgaaacgttgatattcttccttcatgttcatcaaacccaaccagattctcctaaccttaccagcttcattattacctcattatagtcctgtcacttttcatttaaaatttgtttgtttcattaaaacaacaaaataaatttcaagatttatgctttgttaacgtcaataaatcacaaagaaagagaatagtttcagtgaagtttccagtttgtattctatttctatccttatattcatactaaaaaaaatgatatgtagagtttaaaattaagagaaggtagcaattaaataaccagtaactgataagaaacaacttataaatagggttatacaaaaaaaaagagcaactcataatgccttatatatataataaaataagagaaaaaataacctcttcttatggttatggtgcaatgagtaaaaatgtaaaatagaaaacaactttagtatttgtcataagttttggatccaatggtcaggtatttaagtggcatttaacttgattctttagatgaacctccctagttcacatactaacatgtgtctaatttcataccaaacaaaattcataatttcttttatatagattgaaggaatagtaatagacgttaaaaaaatcgaaggaagagtaatgctgcgactgaaaattgaagataatgaaatatattatatgattccaagttttggcatttttaaatgttttaaactggatgactagtaaaagggtgattaaccttaattcactctgccgacatcattaacaaaactaataaattattagattaatgcttaatagtctcgccaccgcattattctaatagataataaatggaatattctttcagacttgaaagaaaaacaaaattctaaattattagagataaaactaacagcggattcttttgagtactaattatatacactagtatagtaaatatttagtaaacaagacttaataatgacaccacaaattataaaaattcgcctgtcatgttagaatgcgaccctaacccggaagattggttaatatgataatacagcattttacaacaaaagcgtctatgtatatcagatgaagatttgaataacgtaacatattttagttttttttgcatccatacattacttagctctttattcaaagtatatctagcaaactgtggaaaattatttgcgaataactccttcgtccttaattttgggaatttgcaaaataattatcaagcggtttttaacaattatcatattcaatattaatacgatattttgaataatatgcacgatatataacacgttgtttaaaaacatgtacgattaaatgcatactgtaatttaattgaatgatacggagcgattttataatctttgctctatagatcgctatcaacctaataaaatcttcaattaaggtaaaatcaacaagaacaaatttaattagtatgttctgacaccatgagttggttaactttaattacttttcttttcttttttttgggtacaaaaatagacacgtactaggaatcaaaccgtataggaacattttctaaatcaggattatttagccaacatatatagtagtaattagaaaaatagatgattttggctatcaaacaaacaaaattagatgattttcgatatataatcttgtatggttgagccgcataaccccatcaagttttaagatcatgactattggagacgcatgcattcatttgttgtgtttagttaggttttttaacggataatagagttttgagtttaccaaactaattctgatcgtaacaattgtcagacttcaaccaacaaaaactaagaagtaaataagtaacagttttcagatataagtaatatacaaagttatgttattttcttatacaaaaattgttctttagtgctaatatatagatatacaccaaatacataaaatttgaactcagactagctagttctagaaattgtagtcgctatgaatagattgggaggtaattcgcttacatagttacatcgcataattagtgtaaacttgcaaaattaaaatgcaaatattattacgtaggagcatacgtgttgtgttgtcctttttcccaaagtgattttaaaactaacgacgtgttcacttgcgatatttgactcataaaactagctttttagcttctcttgtacggattcatacaatatattttggacccactatatggaccgtagtatctagccatttcgtgaattctatacatacgcgtatatgggtgtcggtgtgtgcgaaatatagtcaaagttgacgttggagtttgggagaaggaacatggagctccactggtttctatgttgatgatagttatgccacacatcagtttttgataaatgtagttgttgttaaactttggaagtcaaagaggataaaccataaaccagtttcttaataaatatgattttatatatatcgatgctagttatagggttacaccaaacaaacacatcattttcattatgtaataaatatcgttatggattgttgttttcgtttattaaatgctattgctttgcatagatatactatagtgattataaatataatatacgaaaaagaaagtcttagtatttgataaataatgttatttataatagataacgttattttaataaaatgcccataataattaataaaaaataaatttgactattaacgaaaaataagtgaaaatgaaaacgttgacaagtgggtcagcgataattccctttgtgatggaacgtcccaaagttcgagacgcatcatgtgattgtgctgtgtggtctctcatatgcgcatgtatgatactaacgtgtaattgtagtgtgtggcttatatgggattgtaaatttctttctatataatcgtaagtttctgaagtcgatgatccaaattgactatattttataatcacatataacgtttagagtagtcgacaattatgaacttcagttttctttttccaaataaaacgttgaaatcaataatgagttatcataaattagtttaagcggatatgtaactacgtataaatactaaaatatgtatatgtgtgttaatttggaatacggacatatatagtagtatctagtctaactcatgtaccacgtccatgcaatgcatgagacatataaccaggtaaacctaaaattgaagatgatcttataatggatcataccaaattatatattaacctgatcagcatcaaatcaaaaattataacaaatcacatatcctcaatcacaagaccaaaataattaaactaaaatccaacttacaagatattttttatggttctaaatttggatatctatctatttgtaaactttagtgtcttattttgaatggagacaaaaatacagaaaaacctatcactctatcattcatacaaatgctgacctcataaagacacatcaacagtgtttgtgggtctgccaccaatcatatatgtgtagtatatcagatcttatattatttacatatgtatatatatatatgggggagatatagttattacttacattataggcatctaaattttgaaaaattgtattataggattgagaaagacatatttaaaagacatatatctaataaaagacatatgtaagtgattcttcatttaaaaaaaattctgataagcagaagtttaaagatttcttttaattacgcaaattttaatgattaaaggttttgatgataagcatatgatgctttctttctagttttgttatctatttgaaaaggtttttaatgaatgagatgtttggtcgaatgttcaagtagctttataacaaatagaaacccaaatatgatccaaacagcacgtagtaaaaatcattaagagcatatatagaatgagccacgtagtcataagctttggtcaacgacatcggatagagtactcaatccacctttattttttttgagttatataatgaaattattttaatgggcgattagtggcatattgaatgatgtcatcgtaattaaacgtttacttggtggaatatactgatgattccaaggaccaccgttgctttgagtgtatgacaatctcgtacttataatcttatcattcaccaaaatcaagatcagttcgctttatatatacttctttattcaaagtcaaaaaagaaaaaagaaaaaaaaaaactagcaatgaagagtgaccgtgtagattttgttataagaactataaatttgaaagttcaaaataactcgtttcacccatcaccctcaagataattaacatcacaattcacaatttactttcgagtaacattacgtttctactaaactctaactattgatctaacctaactaagtgattgacaaaataatccgatttagagttataggattggatttgagttgttagaattagtaaactccgtaaaagaaacaaactttggctcactatagctagctaaaattttacattaaattcgtgattattttagggttatccatacatcaattcaaatatattaactaaaaaatatataaaatatctcttaagcttttcatcataaaaaagtattatataaaacattacgattaattgtctttatgaaaaaaaatggtctttatgaaaatataaataggattttctccgaatggattgagcaatttaacccgaattgaccgctcatgctagtgcattcagtgagctacacaatattgtaaagtactagggtttgaattaagacaaagtttattgcatcaaacccattgtatacgatatatggaagttaagtaaaatggtaagacatcaatcaaaatctaagaaacgggggcaagaagtggatagaagttaggtttaaattcaacatgttaagagacatgactcaaaatccaagacaactggacatacacactctatacagttgttaaaattaaggaaattaaatggagaaagaaaatccagctagggtttggtccatgcattagcagtgaaaagagtacgtggaagggccacgggatgttatggactttgcaatggtaggtttttaattagaacatacctcgacttaagaactcaagacatatctatttcgtactttggtctttggaatttgcgccttcccttatttgtgattcttcttgaaccaaccaacctgcctactttaagatttattcaaattaatttgcatgaacataacaattattctgttttgagctttttacgtaagaaacaaagaaaatacaatttatagttttattcactcgtgatccatcaaaccaacacggccattcaatatattgaatattatcctaactttagagatgatacacaacacaagactcgtctccaatgacaaatagccatcaaatgattaattctacctgtaaagcgacaaaaacattactctaaaatatgcctttaaaatttaattaaaagaatagataatttggtcgacagatagataagaagaacataagaagatttcatccgaagagggatttgacaattatggtgaaagaaacattctgtacgagatcttaaaaccatataatctcttatttgtgacggaaaaaaaaaaatcgttgtaaatgtaaacaaaacattctgtatgatcatgtttttttttttttttcaaataattttctcaaatgatatgacaaaaaagagaagtggtcacatctcttatgggctggaacattggtccttataattaaaacccatttggggtatttgtgaattaatgagaaaaagagtaaattgcagcctgcggagaggaccactactgtcaccgtgtgggcacgatttagctggccattcattctcaacatcatctcactatgtattatcgcaacaactgtcttttctttgatcatctttgggtccaatatttatacatatgtgggacaacatcaaatttttaccgtacgaaatatcagaaaaagcaatgaaaaaatgaaaaaaaaaaaaagtcaatgtttgtactttggttggttaatttagtgtctgtgggtttgtggggataacattttagaatcttttcgattttcttaaacgttccaatataaaaatgaaaaaacaatcaaattcttttgtttatagagttactgatttagtatcaggctcatatttgatattcccaaatggcttaggaagttaaatacggcccaattgataaattacatccaaagcctatattttggatgtcaggcccaactaaaacccaaaagaaacgatataattctataatcccaaaactaacaaaagaacggtttcattgtttggcgttttacgccttgcaaacacacacacacattcatgaccatgaacactttattaatgcctaaatatacattttcgtttttgaggaaaaaaaagtaacgaagataagaaaatagaaaatcaaatgatagagaaaagtttgttacagagaagaagaagaaggaagaaatgattctctgtatttctcattaattcacaagtatacaataacacttgttatatagtaagatacattagaccaaaaaaaaaaaaaaaaaaaaaaaaaaaaagatacaataccggtttgcattacagagatgaatcatatggtttagtaaaccatatactctgttatcaaaatcagatgaccttttgtttaggtctcaaacccaacaacacataattttgctttcttcacagtactttgatttttttcttacaattatttaagtagatttgtaaatgaacaaaactttatcctttagggccaacaatactcacgactactcagggctttgaattcggaaataaaatcatagcttgattgatttcaattttaatgccataagacaaaataaagctgttgacaacactcaagctcaatctagtatctaaacaactctttcaaaacatatttgactgattggttttgaccaaaaaaattgactgattggtttgtagtagattttacaattattttgaagtctttttttttttaaaaccaaaactttagccaaagaaagttttgattagttcaactttagaactctagagaaagcatatttttcgctggtttgttttttaacgatatttttttacttagttttgttttaaatcttgtctagataataggagatatttaaggtatatctgttagcaacataatccagatgagatttgtggagagattcaataacaagaatctttactgaatctaaatctttttcacccttaaccctataaaagattcaccacacctcccacaaaactctttatcacacacacaaaaaaatacctctcggatcttttcaatggacgagattgtagaagactcgagcagagtcatggagatagaaggacagaatgatgatctggaccggtgcgtggtggaggaggttgagctaaccgttccaaaaaccgatgatccaacgttaccggttctcacatttcgaatgtggactttaggtcttggtgcgtgtatcatactctcgttcataaaccagtttttctggtacagacagatgccgttaaccattagcggaatctcggctcagattgcggtcgtgccacttggtcacctgatggctaaggttcttcctacaaggatgttcttggaaggatcaaagtgggagttttctatgaatccaggtccttttaatgtcaaggaacatgtattgatcacgatttttgctaattcgggagctggaacggtttatgcgactcatatacttagtgcaattaagctctattataagagatctcttccatttctaccggcttttctccttatgatcacaacgcaggtgattaattttcatctgccctaatataagcataaaacgatttacatttttattttatttttattttctatttcgctttagcatgtattttaatttatatttttagtagatttatagatagaaactagacgatagaatggtcgacaactagatagttacagaattacttcgaccataccaaaactaagagatatatttcttcattaacttttaagtaactaaactgaacttttgtctcattgcaaaggatacttgttggctttggtttgaagtttgtggtgattggttagactaaaactttttgttttgagaccatttttgggcatttgttacaaaaaaaatgtttacttttggggaactttttcttatttttgtgaccatggaatctccttgcagtttctcggatttgggtgggctggtctattccgtaaacatcttgttgagcctggtgaaatgtggtggccaagcaatctagttcaagtgtctctcttcaggttatactctttattttacatttataaaacaatcatcttcatgattagtcttcaactaaattagttcactaatcattgcagtgccttgcacgagaaggaaaagaagaaaaagggaggcatgacccgaatccaattcttcctcattgtccttgttaccagttttgcatactacattctccctggttatttattcacaatgataacttccatctcatggatctgttggcttggtcccaaatcggttttggttcaccaactcggttcaggtgaacaaggtcttggtattggcgcaattggtattgactgggctacaattagctcttacctcggtagcccactcgcgagtcccttattcgctacaatcaatgtaaccatcggttttgtggtgatcatgtatgtcgccactccaatttgctattggctaaatatttacaaagccaaaacatatcccatcttctcaagtgggcttttcatgggcaatggctcgtcctatgatgttttaagcatcattgataagaagtttcatctcgaccgagatatatatgcaaagactggtcctatcaatatgagcactttctttgcagtcacatatggtcttgggtttgctactttgtccgcaactattgtccatgttttactcttcaacggaaggtatttaactctatcaactcctcagaactacctaaattttactttttaatttttttagctgaactagatatccgagagtagtctttggtgaaagtttctaattaatttccttgcaactctcttttattttccagggatttatggaaacaaacaagaggtgcttttcagaggaacaagaaaatggatttccacacgagaatcatgaagaaaaactatagggaagttcctatgtggtggttttatgtgatcctcgtactcaacattgcgctcatcatgttcatatcattctactacaatgcaaccgtgcagctaccttggtggggagtgttgctagcttgtgccattgctgtcttcttcactccgcttatcggtgttatcgccgccaccactaatcaggtatctacttaaaaccaaatatgaaagtaaagaaactgaataaatttctaacaaattatttttgtgaatcttgttttgctaactttataacaaaatcatgaaaacaatgcacaaattactctagaaaaacccttgaaaaaataatatttcattaaaagaagtttaaaagaagttttgattaaaatatcgatattcttttacaaataatatttcattcatctatgtctcattcattgtccttttactatttagagctagagctcttttaccttatttttttgtaagtgacaacttttaatctaacaaggctcctattgctttaggaaccgggtttgaacgtcattacggaatatgtaatcgggtatctctatccagaacgtccggttgctaacatgtgctttaaagtatatggatacatcagcatgactcaggctctaacattcatccaagacttcaaactcggactctacatgaagattcctcctagaagcatgttcatggcacaggttattgcatcttaaaccatcattagttattggtttaaacgcgatataattatatactaacacaacattttcaggtggttgggacgcttgtgtctgtggtagtgtacacaggaactgcttggtggttaatggtagacattcctcatctatgtgacaaatctttgcttcctccagatagcgaatggacatgtcccatggaccgtgtcttctttgatgcttcagttatttggggacttgtaggaccacgtagaatgttcggtaacttaggagaatacgcagccataaactggttcttcctcgtaggtgcaatcgctcctttctttgtctggctagcgaccaaagcattcccagctcataaatggatctcgaagattcattttccggtcattttaggagcaacctcgatgatgccacccgcgatggcggttaacttcacgagttggtgcatcgttgcatttgtgtttggacactttttgtataagtacaagagacagtggtggaaaaagtataactacgttttgtcgggtggtttagacgcgggcactgcgtttatgacaatacttatattcctatcggttggacgcaaaggaattggattgttgtggtggggaaacgctgatgatagcacaaactgtagcctcgcgtcttgtcctaccgctaaaggcgttattatgcatggatgtccagttttatgatcttgtcactattaagtaaaaatcctttagtttttcttttggagttagttggtttattttttatttggttttgtacagagctctacaacagcttgaatattatttatttttagaatcgtttttatgtatatataaaaataaatatgatggattttcatttctatgtacttaacaaaacaaatttcgtcctttctcaacataactatgatggattttcatttctatgtactatattattaagtacaaattaaaaagcctttagttttcatttggagttagttggtttattttttatttgttacatacagagcttacagtagcttgaatatatatatgttggaaggtgatcaataactaagtaatatgaaataagacttatttaaataagattgaagattaaataattacaatttttgtttgaatttagttaataagatatttaaaatatgaaataagacttatttaaataagatttaagattatatttcttaagaattgattatacatgttatacatataaatatatatttattaaacattaaagtttaataatttttggtttatttgattcagtttgtttcggttacagtaaagttggtttatgcgtctttatgtttggtatagtttgagaacaaatttcaatctggttatgctttgatcgagttagattgtttcgattcaattacggtttactcggtcctcccctttatgagtccttgaatcaaattttaaaggctctcttaccaagaatcaaaatttttgaaaacagaaaaacattaaaacaatatgtaaatgaaaattaggattagcacaaattctcgaagttttatttgcatcttacttcaacatttgttaaaatataaaggacgttcatcatttgcaaataaagcagatattttcttaaccaaaaccaaaagaaacatttctatccataaatttagaatatatgtttccttaagattatttttacagtcaatgctaatctgcaacaaacaaaaaagtcttattttgttgtgtatccgtagtatacttccttctataaaaactatcaataaagatttgataccaaaattaacaaaagaagattcattgaaatactctactatatttcttcacccaaatgtctcaaattataattcaatctggtttcattcaggatctcaacacacacaactagaggaagcaaaaacttttctctgtgagtatatattttgttatttcctctgttttgtttcatatttttaattgcaaatcttaagaacggttacataaacacacgatcacacaaacacataacaatccgttttttatttcatgcatatcatagagacaaaaacagaacaccaatcctctgtttctgaagaccgatgaaagactttacagatacaatatcggagtcggagtgcgacgatgagataagtatagttccacaagtggaactaacggtgcccaaaaccgacgatccaacttcaccgacggttacgtttaggatgtgggttcttggcataaccgcttgtgttctcttgtcatttcttaaccagtttttctggtacagaaccaatcctttgaccatctcctctgtttcggctcagattgctgttgttcccattggtcatctcatggctaaagttcttcccacaaggagattcttcgaagggacgaggtggtctttcacgatgaatcctggtccctttagcaccaaggaacatgttcttataactgtgtttgcaaactcaggctctggggctgtttacgctactcatattcttagtgctgttaagctttattacaagagaaggcttgatttcttacccgctttgctcgttatgatcaccactcaggtaaaaattgatagctttgcaattcgtaatttgtaggtttagggctatgtgaactgagagttttattttccctgtttcttgtgctgcaaggtattgggatttggttgggctggtttgtataggaaacatttagttgagccaggtgaaatgtggtggccaagcaatctcgttcaagtgtctctcttcaggtaacggttctttctaatgattctcttatatgttttgattaagcatttctaaaagtatctgattttgattatacacagagcattgcatgagaaagagaacaaatcgaaatggggaattagtcgaaatcagttctttgtcatcactctcatcactagcttctcctattatctcctacctggttatctcttcacggtcttaaccaccgtctcttggttatgttggattagccctaaatcgattctagtaaaccagcttggttcagggtcagcaggtcttggtatcggttcctttggtctggactggtcaaccatcgcgtcataccttggaagtccactcgctagcccattctttgcctctgcaaatattgcggccgggttctttcttgtgatgtacgtaatcacacctctctgttactatcttgatttgtacaacgccaaaaccttcccaatctactccggtaaactctttgtagccagtgggaaagaatacaaagtaacgagcatcatcgatgccaatttccgcctggatcgtcaggcttacgcagagaccggaccagtccacatgagcactttctttgccgtgacctatggactaggtttcgcgaccttgtctgctagtattttccatgtcctgattttcaacggcaaagatctctggactcaaaccagaggagcgtttggaaaaaacaagaaaatggatatacatacaaagatcatgaagaggaattataaagaagttcctctctggtggttcctttcgattttcgcggtgaatcttgcagttattgtcttcatatgtatttactataagacacagattcagcttccatggtggggagctttcttggcttgtttgatagctatcttcttcactcctctcgttggtgtgatcatggccactactaaccaggttagaagagagattcacatgcttataacttggcttcaaaaaccggctatgattaacaaaaccatgattctgttctttgttttgttaggctccgggtctgaacattatcacggagtacataattggatatgcatatccagagagaccagttgctaacatatgcttcaagacttacggatacatcagcatgtctcaatctttgactttcctctctgatttaaagcttggaacttacatgaagatcccaccaagaaccatgttcatggcacaggcaagtctaaatagtttatgtgtgtcacataaatatctctaaaacatttgtcaaaattgttgttgttttcacaggttgtgggcactttagttgcagttatcgcttacgcaggcacggcttggtggctaatggctgaaatcccaaatctctgcgacactaatctgcttccaccgggaagtcaatggacttgtccatcggatcgagtcttcttcgacgcatcagtgatttggggactagtaggaccaagaagaatgtttggtgatcttggagaatactcgaacataaactggttcttcgtaggaggtgcaatagctccagcattggtatacttagcctcaagactcttcccaaacaagaaatggatctcagacattcacatccctgttctgattggagccacagctataatgccaccagcttctgcggtgaacttcacgagctggctcgttatggcgtttgtgtttggacatttcgtgtttaagtacagaagagaatggtggcagcgatataactatgttctgtctggaggaatggatgcaggaactggattcatgtcagtgcttttgtttcttgcgttgcaacgtagtgagattgcgattgattggtggggaaattctggagaaggttgtcctgttgctaaatgtccaactgctaaaggtgttgttgttcatggttgtcctgttttctaaaaccaagattctcctgtattccttgtagcacattatccattacaacgtttgtaatctgtttctataaataaagctgcttattcagtaacataagacaacaaaaagtacatttatagaaggagaagaagacaatggaagtcacaagaatccaaacaaagccaaacaacatttgggtcgttgaacaaactacgaatatcactacaagtcccaaccaatttgcatacagaaagtcttgaaaaggctttacaaagttctgaaaaataatttttctcttctcttttgttttgtacacatcaagatgtctcttccttgagcctgtacaacgtgatttcttgctgcttgaagtaacggcgatcctcttcatccaccagcacaagatttagataatactttacgctgaatttgttgtttatgttcttatgcgttggtgtcagatcgtatggtgtcaagaacactcttacaggtatcgattcgcctgcaacaatggtctcaatacttggttacagaaactagtaataggtccgaatgagttttgtaacatatgatgaatatacatacctctaactggagcaccatccattaactcaaatttggctagtgtttctgtttcgacgtgagtattagctcctgctccagttgattctcgccgtcttatctcaagatccatattctttatcttgattctcaccagaagaaagtatattttcccaaggataacatcttttaggtgatacctgcatcgagagcatacagaattagctactgttttatattttccacatcgctcgaaacccaaagttacgaaaatcagtttatcttacttgcttttattgtactcaaactcgatatgcagacagtcctcgattccaacttccatctgtacaatgggtaagggtccaatggaattaagcgtatctaatgctaataaggaaatggaatatcaaaaggccattcactatcaaaagcaagagcggtaagaaatcagtaaacttgaatcatatcagaactcaccttaatgctattattgattggaggaagcggaacatagttacggacctgtttcaaaccaattaaaaaagagctaagtttatcttaaggactattgtgtcacgaagatatatatacacacaagtgaagaaaaataccacaaagtcctggtattccacgatgcttccagcataaccacgtgtgactgttactttgaggacatacctgaaggaaacagaaacaaaaacaatgtacacaataagcaacaattagggacttaatatattgtttcaatctgatgtcaaagaataacaagatcataccttaggcgcacgttcacgccattgtatgtctcatatggcatctcaacgctcgaaaattcaaaagggtatgtctttctttcatatatttctccaggtacatcaatctcacgcactacatattagatatacaaaccagaacgatagttgggtcaatcaacgactactgatgaaaacaacacagccatgtcataatggaatttttgactcacccaaggaagtgaagtcataaaagtttcctctgtcaaagtacatttctgttaaaaaagaaacaacagagtcgtcgataaacatataaagacatgagttagaaaccagataacagaaccgacataattcacagtatttgcagacctatttgaccaagaagctcaacttttacaccattatgctccacttttttcccttgatatggttcaatgttaatctgagagagacaaattacgaaaaaaaacactcctataaaagcaattccataaagctcctgataactagagatgacaacaaaaccaagtaccttcccagcaatagtttcttgactctggaaaagtggattcataactatttgtccattatctttcttagtgggtacctaacgcaacagagaccaagtagatacgataagaaaacctataccctaaagtccgggaacaattaatccgtaagataaacgtaaagagcgatgtgtacctgtttacggtttttcccatcagtaaatgtgattgaaatattacatgctggcttgaaagctccaagaagataattctgtgatattaatccccaaaaaaggtataaactttagtaacaattacagaaacaacgacactaagcaaataagagaaattttgttgaaaccaacacttaccatgataagaagattcaattcccgatcacaaaaaaaaaactgttcagagcataagaatccaacaaatgagacaaaattagtggcattttcaacttgaaacaccagaaaggtttggtttctaagctagagctgattcaaattaggaaacagagataaaccctaatctcaattcatgaaaaagtaatcaaacaaacaccagagaagaccaaatcaaatggtaatgattcaacgatgagaaaccagacttgatagagaccaaactgagatccacaaagtttagattcgaatgaacaactaagaaattgtgtacctttctgtagtgaaatcaagggtttatttcaatcggagagagacgaagaagaatcgatcaaatcgacgaagattctgacagttgggttcgagacgatcaacagagtcacgaaaaaaaaaagtcttttcttttgtatcaacttgatacacaagcccatttatattataagtaccgatcaaagcccagataataccattgacataaacaggaaaacggctaaaatcaacctgaactatttgcatgcggtgtttttatacccagattcaaatgtatggtcgtttcctatccgaagtgattaaatattcaaattcacaacctgaacttatcaatattaggccaaaatcaacattgacttttatagtgttagtaaaccattgatttcttatgactaagcatatattgctacgatgatgtggtgttatacgggctgatttgcatctgattattcccaaatttgaagctttaaaaactcattttcgtaaaaccaaatttgaatttcatcagagtaacataagaacttgctggatccgagtggtcaaaagtgaaatattctcgtcttgaggttgcaggtttgaatatgcttgttacaattttacatttttttggagatttttgccacgttttcctgtataacatcacatcatcgcaataataacggttgactaacattgttaaagtcaatgttgatttgacctaatattgataagttcatgttgcgaatttgaatatttaatcacttcggataggaaacgaccatacattagagtctgggtataaaaacaccgcatacaaatagtttaagttgattttgaccgttttccctgacataaacatccataaatcttataaacattctaaagatgtcatttttacaaagaagaaaaagaaaagctaacatttttattcaaaatttgaagaaaaccaaattggtaaatgtatagatcattgcttgaacgagacatgaattaaattttctaaaaacatcttacggcaacaatgctccaacttattcagagaaatatgaggtttacaaaaagtgtatattcttgggttgtactctttttggactatacgaagctctagtgaagagtttgcttctattccgaatctccttctgaaggatgttgttgatatgaatcctgagaatctggctgaatgtttctaagctgcgagacaaaaagcttggagatgccgtttatagctgcttggacctcatcggcattgcttggacttccccagacagaagattgagagcttaaaccagtgtactcgcttgcagcaacttgtgtcttctttgaggtagcaagaaccttctccaagtccagttgtgtcaatggtctaggaacctgtgaaatgtaggtaactcaaagatgtcattacttaaaaatcaggtggatgaagggaacattgacattccctaaaaaacggtttatcacttacagaaactcgtttgccctctttctctgcctctaggatttctcggattgggaagtaagctgcttttttgcagagttcaaaaatatctgatccggtatagtcctcacataaacgagctatacgatcatagttaatatctgattcaacactctctcctttcaaaacaactttcaatatttgcgctctctcctggcaatcaggcatcccgatctcaaatgactgaggaaaacgcctcaatatcgcttcatcgagctctgatggtctgttagttgcagcaagaaccatcaccctcgcattctctataaaaccaaaacatcaaacctcagactgtacatagttcacatggctaaacagaacaatgaggacaagaatttcaactcacggtctgtagtaaatccatcccataaagccataaactcggtcttcatattcgacattgcttcattatcagtcgaccgtcgctggccaagaaagctatccacctcatcgataaaaataatagcaggttggagtttatacgccaagctaaacacagcagacactgaagaaaacataaatcagtaacacaaccaatagacaggtaatgtaatctccaaatctacacctaccaagcttctgtgcatcaccaaaccacttgctcatcaaattagaaacctttacattaataaaaacagcttcagactctctcgcaatcgctttagcaagcatagtcttcccagttccaggaggaccatacaacaaaacacccttttgaggacccagtaacttcccataagcaaaaagctcaggtcttttcaatggtagaatcacaagctcatacaaagcttgcttaattgactccaatcctccaatagaaccaaattcaacatctatgtgtaatgggtttattacatcacacgcaatcacatcctgttttcacaataaacaattgaaaagattggattttgaatacgaattcaatcaattagagaactgtatagaatcaaaattacctcgtattgattggtttgaataagaggacgacccaaacgtttggcgatttctcttttgtgctcgagagactttttagcggcgtcacggttcgggtcaagttgacggagaccaacgtagagaaccaagctactcaatgcagcactagcgacgtaaagtatcaattgttctatgaaatttgaatcctttctccctgaagatctcttcatatctttggatttgggaatcagagaagattcaaaacgaaaccctaattcttgaagagcgttcgtttggggttgaggatagggaaaggaagaagaaagctctaaactggaaattacaggaaaagaacagttggtggcattatcgtaatatcgttgagaattgtgtgcatattggtaattgttttggaactggcttaataggccttgtgtggtgtttcgccaatggatctgaaatttaaagcccttcgaaagcccaatttctaatattataagattttgtgaattctgtttttgttacaaaagtagcatttggaaaaagtcgactcctgaaaaatagtctctgtagtatttccttaattcctttatatatctattattcaatactcactacacaattatttatttctagataagatgcaagacatactatagctactacaagtgcaagagtgtttatcgagttatccttacacgtttcgttgtttcatctcccgtgcactctttttcgtactataatggaagtgaaagagtcattgaatgatatatgtaagagatttgaccttagcatgcatctaacacgtcaacttcaaactaaattaatatatcacaaatcaaatccaaaacgtaaaataatgttctcctcgtcatgactatatctctatggattattcaacttcttttactttgatcgtgaggaatttcatatggagataaacttttttttcgtcaatcttgtacagttaaacattttccgctagttaaagaaattctcgtaacggagtatgaaattttttgtgaccacatatcatcgtagaaatttagacaaacaaaattaagaggatgagagactcgatgacttttattgtatttcaataacaatagaaccatgaaaaaaatacgtgcacttacacaagaagcagccaagcaggtggcctagaataaaaaaaggcgtttggtgttcaatatttttcatccatttgatatttttatttcttctctttctacatacatcgaatgtttatcttttaaacttaccaaatggattaaaatatcgaatacaaaagcgccatcaatgatgatcagaaagtggttttggcatgggaatcaaaatgcacgtggctttccgagaaagagcgcgttagacaaacgcagatctaaaagaccacgtgagaatcagcaaacaagcaaggttgtgaacaatacactgacttcaaaattgcgtttcttctcaaccgcatccaccatgttttatcatttattcaccagaaaagaaaaagaaacagagatgttgatcatgcatgttgttaccattcgaaacgctaaatttgtttgaaacaaataaaaaagagcaccaagttcttagccttccacttgtcatttgtcactttatgtttgggttctgatattccatttttatgctttatatatctcaaaccaaacacatatatagttggggatgcattattatccactagaatattaagaatgttgactatagacaactgtatgacatttgagtctttgagaactgcgaaatggattgatcgatgaattttttaaaaatgtagacctaactatataatatcttacaacttttttagtttggccttccattatcaaatagtctaatatttttttaataaataaataataagactaaataatttgggagactgaccaaaataacgaatattataagatcgatggaaaatggtttaggaaaaaaaaaattagactcctttttagtttttagtttacaaaaatacattgatggctgctaaaatacatatatccagacggaactgggacatgtagggatattgaatgacaaaagtactttgtgataagatatgaataaataaaccgagcaagttgataatatattttgtcaaattcgttgaagctaaataaatattacactgtgaaaaataagtagaatagtggtccatacattcttacagaatttactatatgttgaattcaaatctctcacaatgctaatatcaatataaatacgatagaaaatgaaaatgacatgttttattcttcaataactggtgtgtaaaaaagtatcgtatgaaaataactgaaagtgccattttataccaaatcatagttgcttggtgcgtatcgtttggcataaagtgaactataatttagatgtgaaggtccaatattcccaagaggcgaagacaaaaattgggtagacttaaaagagtcaattgatcaaacatgaactgaacttgggtaagatcatactttcaatcattagaaaggatccaccgagaggtctagctatgaagataggcatggtcaagctgagaaggttaggacgtaattagtaaatcttggttatagatagatagtcatatggttgatcttgctttctcacacttaacaccatcagttgaatccaatgttaaaaataggtaattctcagatgcagtacctcataaatgttgtaaaaaagtcataaataattattcaaaggaatgcatggaagaagtgaattacatatttttaaaaaaaatataggaaaagaaaagtaaagttcgtacaaaagagagtagtattattctctttttcttctccatagggcctcgtaatatctacttagcatacgaggacacattaatattcgatcatgtatatatatgcctacgactacgagacattcgaaagttattgtctagtggtttgtattcacacaatttccgtgttaacagttttagttccatttccttcatatagagatctaaatgatactactacatatcatttagtttaaggaaggacgatgtaagaaaataaaaatctaatatacgagagaattgtaacacgttcacttcataactagctagctagtggtacttaccagttactaccccaactctagtcaacactacattaaatactataatgtgaacaaaattattttgaaccaacggctgttacagtagtttattcaactttatcaaccaatgcattaaattgaacatatttctggccgattgaaataggttgggaacgatcttataaaattcagtcttaaacaagtcttggttttcaaaaatgcaaattcattcgtatgttcttggcaaaaaatggtttaattggtttgatctgatcttatcctatcacgcaggattggtgtaatataacgcaccaaattcggccaggaaacgtccgaatataatagtccaattctacttatgaatttgacgtgttgacgtaaagaatcaaaagtcgaacttattgaataatttattcgactttaattttcttttggggttttggtatgtttgtgaccacatggtatggagttgacatttaccattttcttgattgactacctaagaaaattttaattattattcacattatggagagttatgtttccatattttggctttaatattaatcatgtggtcaataaaataaagaggatagttatggcataagattgtccataatgtcatcatcttgtttagtaaaagacagtcacatgaaccggaatctcggtttaccaaacttttcactttttcagtttcacatgaatgtcaccagattcattgagttaaacatgtattttaatactataaaaggttaattagttatgatatcatatttatgacttttaaaagacttttgatattgtaatctgtaactagttataagttaaccatctcttataattagaacaaaaaaaaacttaattatggatgaactgattttaatggaaaaaatatttagaaatagaaatacattcataatttatatctaagtctaaccaaaacacaaatctaaagttacgaataaatcatttcaagtgtaattttactaactatttgactcctaatcttatttcatcacctaaactcctaattgttatatatttatgagagataatgacatttgttcctctacatactaatctaatggttgaatagtcatgtgtttgacaaacttataaagtttgatttttcgtatgtgacatttagttatacatccataaattatataactttcttagtgagatatatgaaggttatatatgtggagtatattcttaataattaattgttaccctactttttttcttttcggtgacgtaacttttataaagtttaaccccttaatgattattgtactacataaaccccaaaactcttttctcttttcttttttctttgtgtagtactgatatcaagtcaacgtgcggtacataagaacccaagatcccataaataaattcttcttggttttaagctttcttcttcatctctacttctctatataactttgagtagtctcaccaaagtccatttttaaaaaatcaactataccatttttagctattctttggtcttcgtttcgtgtaagcattcaatgttttttttcttttatctctttgggaaacgggagtgtggtcttcgtgtgtgtgtccctgtttcattaaccagttacgtttcattctcttcttttcttacgtttactacagatttcagaaagaaaaaaaagtgtttgaagcaaaagagattaatggcagattcttgagtcattaacttttttctaattcttttaattagatctctgaacataagagataacttgcgtcattattctatggttataaagattatgcacttgattaaaataatctctagttttcctttttctcatttgctaagcaactgtttctttttcttcttcttcttcttcttcagataattatgcttgggagtggaaagcaagaaaaattctaaactttatataaaattcccagaatcaattgatccaaaaagaggtaccatttttttcctctcgtcattttccataaaagtaagcttttgattgatatttaattattaaaagaatctgtcttttgggtttttgttttgacaaatttttgttttttcagtattgtcaaaaaagataatcaagaagaagaaactctgtttttttgttctgttcttggaaaaaaatgaggagtatgatgatggagagagagggaaggaatgagatagaaagagaagtaatagatgacttggaagagacgcaaaacgaaggagatgatttcaagtcaatacctccatggaaggaacaaatcactttcagaggaattgttgcaagtttaatcattggtataatctacagtgtgatcgtgatgaaactaaacctaacaacaggtttggtcccaaacctaaatgtctctgcagcacttttagcctttgtcttccttagaagctggaccaagctgttgaccaaagccgggattgtgactaaaccgttcactaaacaagagaacactgttgtccaaacatgtgctgttgcttgttacagcattgcagttggaggttcagtgaattttgaccagttgttttgactgtttgatcagtttttgtttggggttggatcattgttttgagctatgtgtttgtgtgatttgcaggtgggtttggttcataccttcttggtttgaacagaattacttatgaacagtcaggaggaactcacactgatgggaattatccggaaggcacgaaagagcctggaatcggttggatgaccgctttcttgttctttacttgctttgttggtcttttagcattggttcctctaagaaaggtagagattatttaagattctgtctcattgaatttgtagttagagatgttgcggtaggccaaagtttgcgggtcagtccggcccatataagtgtgatatgggcctggccaaacccgctatagccaacataggacggagattaatattggccaagtttggactattaatcccctgttggctggtttgggtctacattgctaagtgttcattatgcttctttttttggttaaccagatcatgatcatagactacaagctgacatatccaagtggaacagctaccgcggttttgatcaacggtttccacactcctaaaggcaataaaatggccaagtattaactcgattttttggcagtctcttagctctctttcaccactaattctagagtttttgatttaaataaaatctgtttatatgcaggaaacaagtgtttgggtttgtgaagtacttctcatttagcttcatttgggctttcttccaatggttcttctctggtggtacagagtgcggtttcattcagtttccaactttcgggttagaagctttgaagaacacgtgagtattattcttagttctattgactgtttatatatcaacagatagaaacagaatcttgatgatcttgaaaaaacttgacagattctacttcgactttagcatgacatacgttggagcaggaatgatctgtccccatattgtcaatatatctttgctttttggcgcggttctgtcttggggaatcatgtggccactcattaaaggtcttaaaggagattggttcccatcaactcttcctgaaaacagcatgaagagtctcaatggttacaaggttccatacaaaaaacactagtactctacaataaactccgccctttctttcgcgtactttcgttcttaatttgttcataactctttaggtgtttatatcaatctcattgatcctcggagacgggctttaccaattcatcaagatactttttaagacaggaataaacatgtacgtcaagttaaacaatcgcaactctgggaaatctagtaagtatacaccaaaaaaactatagaaaaatcgtcatttcaataatgtattatctgatgttcaaacgtaaatcgcagattcggagaaagataagcaatctattgcagatcttaaaagagatgagatctttgtaagagacagcattccattatgggttgcagcagtaggatacgcagcgttctctgttgtctcgatcatcgcgatccctataatgttccccgagctgaaatggtacttcatagtcgtagcttacatgttagctccatcgttaggtttcagtaacgcttatggagcagggctaacagatatgaacatggcttataactatggtaaagtcgctctgtttatcttagccgctatggcagggaaacaaaatggtgtagtcgcgggacttgtcggatgcgggttgataaaatcgattgtatcgatttcttctgacctaatgcacgatttcaagacaggacatttgactctgacttcacctaggtcgatgcttgtgagtcaagcgatcggtacagcgatcggatgcgttgtggcgcctctaactttcttcttgttttataaagctttcgatgtcgggaaccaggagggagagtacaaagctccttacgctttggtatacagaaacatggcaattcttggagttgaaggtttctctgctttgcctcaacattgtttacagctttgttacgggtttttcgcattcgcggtggcggcaaatctcgttagggataggttaccggataagatagggaattgggttccattaccgatggcaatggcggttccgtttcttgttggagggtactttgctattgatatgtgtgtgggaagtttgattgtgtttgcttggaatatgagagatcgagttaaagccggtttaatggtaccggcggttgcttccggtttgatatgtggagatggtctatggattttgccgtcgtcggttcttgctttggccggcgttagacctcctatatgtatgggcttcatgccgagtaaatattcgagttaaagatagctttttacgagtttttacttttttgtgtagcgacaaaaaaattagctaaagaattgtggaaacaaaaaaaacagtttatataggaattgtactttgtaagtttgtatacacaataaagtaataaactagtctctcaattctatttaaaaatccatgtcccaagtaacttctccccaaaaagaaactcaatattttagaataaggtcaaatggaattaaattccagaagatatttgccaaaatagtgataaaaatacggttaaattggtatatgtttttactgacaaaaggcaagattcttctattttaagatttatttctttctttgaaatctgctaaacgttgttagatcacactttatgtgtctgtttttttttttgtcaattcagcacacgtcatatttgtatttttcaaagtaccaaacgtggaccaaaataaaatctcgtcaaaaatggaaaccaaattttaatttgatttgcacaaagcccaatttgatttgtttaaagtcaaagcccaacttgaccaaaactacccctgctcatccaaaccctaagcaaaatatcgagggtagtttcgtattttgtttatattgaacacaattaagtaggtgaggaacctctatttcttccgtcttctgctgatttctgagcttcggagctatcgaaaattctgaatctgaaagggtaaagatcccatctttttccaaaaatcgataagggaattagtatattcgttcattcatcatcgttctgatgatttcgatttgaatctgtatctagtactctgtattatcagaaattttgattagtagctttgatgtgttcttgttttgcagtttgagtgaagatgtcttcagatcggaaggttctaagttttgaagaagtttcaaagcacaacaaaactaaggattgttggcttattatttccggcaaggttagatcttgtctctttgtttgagaacatctctgttttttgacgaacttagttctcttccgccattgatttgtgtcaaagcttcgattttaaagttgtagagttggattgttactgttagaagctttaatcgagcccctcgtagatttagttgattctatgttaattcacactagagaaacagagtttgtgtctactacttggttatgatctgtgattgttgcgttctttaggtgtatgatgtgactccattcatggatgatcatcctggaggcgatgaagtcttgttgtcctcaacaggtataaaacttgtgtttctctctgattcaaggcggatttgaagcggctcggtgactgtttctgttctgatgtggtgttttgtttgcagggaaagatgctacaaatgattttgaagacgttggtcacagcgacactgcaagggacatgatggacaaatatttcattggtgagattgattcgtctagtgttccagcaactaggacatacgttgcaccacagcaaccagcctacaaccaagacaagacaccagaattcattatcaagattcttcagttccttgttccgatcttgatcttgggattggctcttgtcgtccgtcactataccaagaaagactagaagaagccaaaggcttgctttgatgcttgtcattgaatatctctaccgatctctgtctatctctcttgcgaaaaaaagtactgttactgttgtttggtcttagactgattcttgttgaaattacgaaccaaaaatcattgtgttgctactagttttactgtgttgagtttatgatgacagtgttttcgactttgatgatataataaaagactgcttgatttgcccaaacttaagctgcttgataaagaagtgagaaactggatttctacaattgaacaaatatgtttcatacaattgtggaaatgacagtttcagactcatccgttacattttaacaagaatattgccttgtctttttcattagtactacaataaaagcgataaggatagtagtattgtgttttctctgtgtgatgagtaaacaaaaagaaagaagaaagaaagagaggagtgaaaacggtaagaaccaaaccgcaacaaactttaaaccttgagaaaacgcagccagacgagtgcaagggcttttgtgtagtggaaacacttcatttctggctttgatcgacttcttcatcaaatactctgtgattgagaaagagtttcaatcttgtcgtgaagcgtcacgacaagatgatgggcatcatcaagaagcttccagacattaagctgaccagccatgctattacactcttccacaatctcatccatactactgtatttctgtatgatcagtttcctacgtagtactaacgccgcgattgcatagagcaacaaatcatccgttggcggtgcttgttgccggattctgctccatggcgacttcccaactccagctctaatggcagcttgatcagcccacatcacttcccagagacacagcgtctgttcaaaactcaactccctcctaaacataacaagcaccatcctgtaaacgaaactacaatcttctgcttgaagattctccaagtgtttataaagctgagagtctttattttttatgatctttgatacaatgcttagctgtctttgaatccctgcttcgtcaagcctgaaattgtgccgagctttcttcatgaaaccaacaaagcaccagaaagcctcgtgatcctctgagatcacagcgaggattggagataacagatcactcattccttgacagtagcctatctcaggatcatacatagcataagcttcaagaattgcaactaaccgtgctgcgtggtatagcctgcagctttctaagtgatcatagtcttttaacccgacagattcggctaacctgcgagctttgctttctgtgattgctgttgaatatggagaataatttgcccattcagaatccgctcgtaaagcatcaagccggatgatacgttgccacgtagagaaatcttcgtgtaccgcaacttctacttggacctcagaagcagcagctacaagaagagaactattctcttcagaattgttattactattatcctcctccggctttttttcatcgctgtgtgcaaaagatgagagtagtaaaacatcttcattatcttcacaagagtctgtatcagaagaatcagtgttcaaagcagaaacaacatcttgacttgtcattggtcctgtaatcttataatcatccacgaaccgaacacattgactgtttgcctcatcagatggaagctcctcaagattatcggtacttccattaccgcacttgagaagcatctgacatcgtctctgtagtttctcatactccttcctacaattcaaagatcccaacaatgaaattatctatcagcatctaaatacaaacattgactatatatagatcaaactaaatactaaccttttttgagttttcactgcttctctttcttcactagtactattcaaatcataactgccataagagaaaacaagttgagaatctcaacagagtgaataaaaataaccaagaccataaagtcaaaacaagaacaaacactcacactccgagtaagaacagccacacttctgcacgaatacttggatcaacaccctacagaaccataagaacataaatcagaaccagtaaagagacaagtaagaaacaagacatacatatggtccttgttcaaaagactcactctgcttctaactttcttcaggaatccaactccaccatcacggagttttccctccggtgtaaataaactcctccattgatgtggcgtcaatgcccattttcttttccggcgagaccacggtgatttccgccggctataaacaagtcaagagtcaacattagaagcttttacaatatcaaaatcaacaacattgattgccaaaatcaatctaacagcaaaacaaataaacaatccgaacaaacatagaaacttgttaatctttatcatactttccaagttgtgaacattcaatccgaactctattattggatcaaaaaagagaatcttttagatgccatctaaaatattccaaatctaaaagctttaggctactcaccgatcggaagaagtaactgaagacggagacgaaagattagccacaaagagaaccgaccgtagatggatccacgacgacgaagacgaagaagaattagaattcgaataagaattagaattagaagaaggaggtgaagaagaagaagaagatggtaacgaagaagaagacgaagaaggtaacgaagacgaagaagatgaattcccagaagacgttgaagtatagcttctccgtaacgccttcatctgattctctaaaaacttaaacctaaaccccaaatcaaaatcaaaatcgaaatcaaaaagtctcctgctttcatcaaagatctacacttttaataaaacccattatcatcgacggtgaatcaccgccggaaatttcccggtgataagcaaaagtaaacctcttcctcctcctcctccgccgtagcagaaatcaagcatgagttgtttaagaaaagaaaaggacagatttaccctcaaatctctctcactgtgattgattactctatcgtcttcttcagcacgccgaaactgaaaaaaagaagagagacgaagaaagaagacgaagaagaaggaaaaaaagcaaaagcatccaacaactgaagcctctctaaccactttgctctttctttttcagctcttgtctttttttttttactttaagacattaagcgcgtggactcgcgcgtttactttaatgctacacgcgctttaactctccacgcgatttggtaggtgaattttaattatgaaactgcgtcgtttcgttttagtcttcgttggtggatttttacgcgatatcgggtatgaccaaagaggaaatgacacgtgtttactatggttaaaagctttaaatgtcaaaagctaaaagattttcattacgttcccactttaccgcggcttttaaggagcataatcatgattagttttgtagattttctagtaaaatttttatgtctactagagaacaaatctaatcataagaatgttgacctaatggacctacgtttactcaatggtaaggttaagaattggattaacgatggttattgaatatatataggtgtttctaaaatactctattttggaatagtttttgtttggtaggataatgatatatattaaatttccatagcaaatactaacaatatagtcaaagggtttatcttaattaggattgatttttatgatttgatgttatagataacagaataacaaaaattaattcatttgacaacttttcatctatattaatgagttttcaattcgaaattattgatcatatggagaaaagtgggcaactaaataattcaatatttatctaaataaaaagttaagagaaatttataagatggaaacacaagttaatatgatttacatgtagatgttttcaataaacaaaaaaaaaaggaaaagggttagtaaacacattcaaatggatcaagttaaaaccgattagtaataataaatgtctatgaaccgagtcaaaattaaactttgaactaagccaaacctaaacttgtttatagcataattataaacaaagactgaaaaggatttgattaatatgatgtttattgtttattttgttgtgacttgtgagtcttactaaagtgtaacggaataaagagaaaaaataactataccttttttttttttgactgacataaagccgaagatatgctattaaatctagccgcgtatgtcattaaccaaatctggaatatagttggtattaaattacaaaagcatatggaaaagaatttattctaataataaagcactactagtagtagtattttatttatatatttatgaaaatatacttgcatgttcttggaactataattgtttttaaataatattctgattaagaaagttaatgagagtttaaatgaggaaattaaagttcgttggatggagaagacatcaagcaagactagtggccaagttgtaggtataaaataacattattacgttctagggataaacatttggttgcttgaaagataagttatgtctatgtaaatgcagaaattgcaatgataagtttagtcactatggatagagagagagagctttcaagagcagaaaacacgacaatcgataactttgtttatgtgtctctcacaactagcaatgtctaccggcatgaaatgtgggtattatgtccaaatctaagcgtttaaatggttgcactggaagaaggactaattgcttaggaatttgatcactatatgcataacaatggtataagaaaactatctgtgttcttcacaatatgtgcatatataaagaagtagattaaaacttctacttatacacattgttctcatttgtggtctagatgtgtataagcatcttctcttatcctatcatcttgtctgaaaaatattctgaatcattgactttggtgattctttagctgcatattctaactgaagctgttcatcatttgtaagtttccatcccagagcacccaaattatcttcaacatgtcttaccgacttgattcccggtatcggtactgtccctttgcatatgcaccagtttattgcaacctgtaacaaatgataactcgttcttacaagtttcaacacttcttcttcccaagaatcttatgaaattgaaataatacctgaggcatagtctttcctcgtttctttgcaatctcgctcagtgctaaaagaagaggttctaatccaggaagaatttgtcggaacagcaatgatctgttaatcagtgttcaagcatagttaagaacaagaaacaacggctagttttgtcgatctttgagcaatctattagtactttaccgtggaccagtgggaagttttgaagaggagtatttcccagttagcattcctagcccaagaggactataagagattaaacgaatcccgagctcgtcgcatatactcttgatctctagttgctcttttcccatgcttagcaatgagaattgcacttgggcagaacataaaggaacccctcgagttttaaggtaatcatgaatcttcacaagctgttgaggtccatagttactaactccaacggctctaactaaaccctgagaaagaggaacgcttatcatcaactttccaagtgcttaaatggtcattaaagagatttcatattttcagtagttttctaagcaaccttttcgtacatttgcactagaccatcccaaagaacaagctcttgtagaggcgcgtagcttgcagttgaccagtgaagctgtccaatcccgagctggtctatctgaagccggtctaaagaagctctaacacaacaacagaacatcatccaatttatcatcaatctccaataaaccagaagtgaaaaagagagacaaagatcaaaacttagagagacctgcaggcattcacaaactgtcctgaagttaacctccatggataagctgcaaactttgtagctaccactacttcattttgtttccctttaagtcctgacatttgcaaccattaatcacaacacacaaacccaccacagtaacttatcaagacagctccaggataatactttagaaccaactaaaggcaattttttccccaaatttctgaaactatctattgaatcagaatctttagaccagtaacaaaattgggaaaggttaaagagaaacacaccttgagattctttaatgaatttccccaaaagtctctcactttggccattaagcctaccagtgccataagaatcagcagtatcaaacaaattgattccattttccaaagccaattcaaaagcttgttgaagctgatcatccatggaagtctgataaccccaaagaagctgattaccccaagcccatgtcccaaaacccataggagaaacacttaaaggacccattttgacctaaaaccatcgattgttcattcaattgcattaaaaaggagaacactttgttacgtttgctttatatgcgtattgatgtgttaccttttgccatggccagaaaagaggaagcttgagaggcttaaaggtggttatgttggaagtattgtttgagcagtttatgttagtgaaggtctttgtggttgacaatgttaaagccattgaaaatagtggagaagaaggctctcaagcctttcttctgctgtttctcagtgtgtggatgtttttgaagataaaactgtcaacgctttttctgggaaaatttaatttaatattcaattactttatttcacataatacactctaaaatcaatttaatgttaatgaaataaattgattttagagtgtattatgtgagatgggcaaagaaaacaaaagtaaggtttgcctctgtttatgggtccagtccagtctcataatagaattttgtggaagcccacccaagaaactacaatttttccccaaaaataaaataaatagtatttttggaattttaatcaattataaaaataaaatgatgttgatttcgtttgtttttttttcaaataacattttacggtgttttaccaccatgcaaaaaagtatggacattaataatattttaaatctatttgtgttaattcaaaattttctaataatataaaagtggtcgacaaaaaaaatattcccaaattttctgttgtttgatcaaaatatgggcaaaattaacataatttttaacaattttatcatggtgtcattttgtaaacaagcttttaaaaatgctatttattggatatgtcgaaaggaaaatagaaagaaaaaactatgttcttcgaaaagaaaaaaagatgaatgtcgtccaaaggaggtcttacaagaatctgattacatagcaagtaataaaacatgcgcaagatgaaaatccactattatacaattttgttacggtgtcattttttcaaccaagttctaaagagatgctacatagtatattagataatatcgtgcacatatatgcggggaaaaaaaagtcgaataggagaaaagaagacgaatgtccaaaggagacttattacaagaatctgattacattgcgagtaaatatatagaagtttgcatgtacgtatgagactatgagtactattgagtttgtgggaattttatatatgcccgaaagagaatgcaattacagaatgaacaaaaaacgaaacaattttgggccactaagacttatctccagcaattggcacaattcgaatatccaaatacacacaatatgaatagataaaattagtatgacatccaatatgatactctattaagaaccaaaacgaatacgaatagataaatatttatttcgacgtgactacatttttgtttgacatcaatttcaccgacgacgtgtcatttgcttttactatatatagtttagtatttttcggcagactttaacgtgtagtcaaaacgacaaaaacacatttagtttagaagttttacatgacgagctaatgcgtgtcttgataaagtagctctaatcacatccacgtaaaaaacgacacctatattcaattaaagtaaggtttcattttcacttgataaaaagagagattttaaattcttttgtagtgacatgttatcattggcgactcgacgatgatgtaattaacgaaatagaaaacgtttgttcgttttttctcggagtagttaaaatctgaaagcctagattgtgtgggtggagaacctcaatacatataataaaatgattcattgacatatatagtttattaagtcaaataactttaagcaatatattcaaatgtttggttgagagtttaagccactcgaaacacaaatctttttttaacatgtgggatgtggcccataaacaccaaggatgcttggtcattttctacgaatatgattcgaccttttgtatcttttccaattcatctaaatttgttatgataacaacaagcactagggttcgatatttgcttattacgtttgggttataaatagttttgatagaattagtactgaaattcatcttgtctgatttcatatggtttggttattttgatgtttctggttaaaatcaaaacaatgtactaataaatatagaaatccatatcctcataatactagtaatattcaaataaggattatagaaagaaaaaaattagtagggaagcagatcaatgttcatcaggttagattttacgaattttaatttgatgtaagtttcgtttgtttctgttagtattcaagaactgtttggttcataacttcatattgaatatcggattacgtttagtgatttgggtttagattctattcaaacgtcaactcatgattaagagtaaactaatcattatacaatcttaaacctaacgacacttggtttttataatgcctttaattctctgccttctctggcgtgtatggacactttgacaaaaagtcgtcgttcgcttttatatcaagaaaactcaagagcaaagttataacaaagagcacatgattccaactttcttggtaatccaacggctattactcatccatatggtcgtcattggtcatatcgtgccgacatgatggtccgcaaactttggaaccttcttattcttttcgtaattaattttaaccgaacaaaaccgagctgatgttaacaaaaccaatcctggtctcattttgtttctattgctaacacataatactggaataacgttttcttatttgattatgaaagaatttaaccaactaaaaaaaaatctgcataatatttatcaagacgcaaaaatgtagaatttgtatttttgtctaaaaatatatatattaaaaaatattatgcaaataagtgaatatacaactgtgtattttagtatatatttttcatttttcaaatttttttttgcaatttatgtcattatactgtgttactaatcgaagtatgagattgaggttatgcttgtgttttcaaaccagttcctaattgaaccaaactttatccgaaattttcaaaaccaaaccattataataagatgaaccggatgagaaccaaaaccaaagtgagggcaaataacccacgtgtcatctagttattggacacgtgttgatcggcgtcgtcgtatgcacctgaggagccgcgacatccgcttactcggcaaaggatttgtttgtcattttgcgcttaggttacgttacctgacgtgttatacttatactataagacaacagttcatgggtccacgtttaatgacgtgtcagagtctttgaccattcgcttcgaagaaaaaattcatggagatggaatttgaatttttacacgagcatgttcctatgaataatgccttccaccttgcctctaacctcaagccacatcttttcttttttaagtttggttaaaagcaaactacaaaattttcagaatgttggattgaatataaatatgtattcataaatcaaattggtttaattaatagctaagaaggcaatttaaagtcttctaatctttattttgcttggtgggattatagtacaattatgaatgaagttggttaaaagaaaaaaaaaaaacatcaactttttgtgtttaacttatttaaaatttataaacaagaacctaccccaagtgatagaaacattataatggagtataacttaaacccaaattcttgttttcaaatatttaatagcctgttgaatgatttcggactaatgtgattatgtgaataggcaaattctcgaatggatgctatatatctaacacgttatatatagtatatatacatatacatttgattttgtttttaatacggttttataccttaaagtatgacttcttaaattgatataaagtcaaatatattatagaaaaactaaacaaaatgattacgttctaaataatagaatttaaaaatatgtgtttatgggggatgtaatttcactatctctaagattaggtataggtttgacctttaaatctttaatcgtgtttatccatttacctttagtcacggtggccaaacatcaagaatgaagtaatgaaacacacattacttcaatctctattcagattcttttggaaaattttgacaatttattggccacgcttccgatttttttattataatgttaaagagagtcatacacataatatgtatacttttttatatatgattaattatccattcaatttaccgagtcgttggctttttgaaatgccaaaggggccttgtgccagacttgccagctgatcaaagtaaaacagcaaccaaatctgatttcatatgttttaagcttagcttagaaggtaataatgtaaaagtaaaacagcaaccaaatcacaaagatcagtattctttgaggttggacaattgcttgttatcttaagaaaaacgtatgaaagtttttttggattttttttcaatcctaacattcttttgatagttaattagataattatttatattacacatacgagatcaatatatttctaattgacctctacattttttgtaagctatggcattaaatattaaacaacaccaaaatattttaatgcagaaaacacaagatttttttttgtcaagaacatgtttttcatatataataaaagatacagaaaaagtacaatagtattttcatatatcaatagttctaatatataagtttttaatacaagctaaacaattaaattagtttccaatatacttaaccagttaaagatagatacttaaaatcatatatatactctcttgtttttacatcatcataaatataggtgataaatgtaattatgtatcaactaatggatatacacaaaaaaataagacataaaaataatcaggtggtgtaaacaaatctcctaaagtcggtaaaaccctaccctaaatactcctccaaacacgctcccaatttttaccacgtcaattccacctcattcatcaattaacaaaatagaagacaaagcctttggctctctttggaaatgtgcgcgttaagcaaaaaaaaacctacaccaagtagaagtagtatataaaagagacgtatatttcctaattccgaaaccacctcctctaaaaaaccaaaataaaaaccttttttctctcttcatgcttagcctatcatgagcaacatccctagatctctcaccgactcagatctttctctatttactctcataatctcttctgctgttgatccttggcctttttctatcaccgttttttagcatcaacaaaactcaccggaaaatctctaacaaccaccaccgtttaacacttctcttcctcctcgtcgatcgtttttaaaccaatctttgtgtttcttgtttggtttttcggtgaaaaagattccttcttcttaatgggttttgaagaaaaccaaaaacagagtccaaaacagagtccgaatcatatcaagcatatggtgttcaagttccacttccatgttcctcatctccacatactccctcatcatcaccaccaccaccaccatgatgttcctaaaggctgtgtagcgattatggtgggacacgaagacgatgaagaaggtctacatagatttgttgttccgttggtgttcttgagccatcctttgttcttggatctcttgaaagaagctgaaaaagagtatggattcaagcatgatggtccgattacgattccttgcggtgttgatgagtttaagcatgttcaagaagttatcgacgaggagactcatcgccgtcatagtcacggtggtcacggccacaacaaccataaccatcacaacaaccatttgcgatgtttctaaagatctataattcgatttgttggtttggtttggtttgagatgtcattggtttgatttgacagatttctcttttgagattgattagtttgcttaactttttgcttttagttagtttgatgatgatgataaattgataatgatgaaaaaatcaaaagagcaattgaatgtgattttttttttttttttttaaatgtaatcgtgaggaataagggaagaatcatattatttgatttttattttatggaaaggaatgtgaatatttttttcctttctttattttatttgattataacgcgttaaatttgggcatggaccatgttatttgttgttcttttccttaagaggattgcttttctcgttaggatttgcattcacgccaagtgtttcgaatatattaatctcttcacaaaaataaaaactcaagcacatctaagaattaccaaacaagcaaatatattttgactttgattgagatccgtcaccaattatatctacctcaattacggcagctttttagttttgtcaacttaattactgcaattaacacggccaattcaaatatactatatttaatttttgaccaataacttcaacatagaatcggtttagatactaaacatgataccgtatcgaaactgaattttgacaaaatatcaatcagataaattgacgttatgatatattacctgaaaatttccgaaattttcgcacatctaatttatcatattagaaactaaaagtatgttcacattagggagttattaattggtttatttttgttactagagtttaaagattactgtaagcggtgagttcttctttttctccgtgaccctatcctgattagtctaaaggtttaattatctctcactttctgattattcttctgcctctatcacgatgaaactatgaatgttttgagtctttcggcgaaagatgatgttactttatgtatggattgaatctgttttagaccctaacaaactagatattgatttagcctttaatggtaagagattatccactaatagcatgagttttgatatcctcaccgatatttcgagtgtcagggatctagttattggtagtattatttggaaggtaaacatctcaacgtttgtgcatatatagattattattccacgagagtttctcaccataacatttttttttgtttacttctgctacaaggaaatctggctatacaaacaggaaccatcgcttctctttcgtaacctatcccatttgaatgctacattttatatatctgatcttgaactgttgccaactcttcttgattggtaaccaagtgctttatactctttgatatatgcttatcattatgtattctctagtcatcgtctaatgttttttttttctttttctttttcgattttgtttttgcaggaagtggctaagaactatttcttgaatttagcaaaaaaaaaaaagaactatttcttgattgataagctgaacaaagaacaaaaagtgcctcagtgtttggcatcatcgctcaagtttgtggaattcaaacgatcagtcctaaagtatgaaggagaaacgaagctagttgaaagccactttagattccacacgtactcttaagaagagaaatcaggtgtcctgtctcctgtgtcccataagcaaaatattggtggttgctgctcatgtatatatatatatatatatatatatattctcactgattgttcaagcattatttagatatttatatatgtataacccgttgactaaaagaaaaataatgtttaaaataatcaataaactatatctttgtgtttgacttaccagtcagattaaccgtggaatagcggaatttcgaaagggttggcaacatttcttgagagctgcccgaatctgaaaaccctagtcgtggtagtaaatctgctaaaccctatgataattttctcatctctatgcagcttctgccatatcatttgattgtatgttctcctctcctttttcctcagctattggatagttataattattactaccaggagctacatctcgaggatatgaatcaactcaagtgtttgcaatcctctgtcgagtttgtggatttcaaacgattcaatggacatgttgcacaaatgaagctattaaggtacttcttagagaattgtgctgccctcaagaaactcactctacatttggattataattcaacagaagatgaaaccattaagaaactcttgaaaatcccaagagccgcctctacgaaatgtgaggttgtcattgtcaaaatgtagagaggaactgttagatgtctgtatttgactggtaaaatcggttgatctaaccgtactcttcaatatgttacttctcatttgggtcttatgaataaaaatgaatgaaactatttacgtactttgctttgctggaaacgtcagttattattcttcgaatcaactggttttttttttttttttggtgattttgtgtgaagaaagaataaagctgtgtgggattaggcgtagtagtttcattattacctggtttggtgatatgttgatagcgtaattcttttgcttcattcgtttccatgttcgtttctatcagttgaaaaacgttaaaaatctcttagctagtttaagttgagcctactaacataaaacttaaggtttcaacaaaagcagattatattaaaactatttaatccgactaaaccgtatctgaccaaaaacataatttaaaactagctaagttgaaaaataaaatagatggaagtaaaacttttgattgaaaatgtttgattaatttgtctacttagggagagtctattcatttttggaagtctttgcgtatctatcttattgaaaaaaaaaacaaaaaaaataaataaatgacttgtaaattataaggaaaatgtagaaatataaaggcaataaggcaaatattaaaaaatataactatttatataacagagcatattttatatgtgtgtagagtttccatgtataacaagttagaatgatatagccaaagatcttaccaaaagttcaaagctactagataagatcatattagctaaaataaaataagaaatatagcataatttatttaatggaatgatttataaagagtgatgtgtaagcgtttgattgatttattacacatcttataattccatctccttagatctcttcccaaataaacaaaagcatttagagttgtgagaatctgtgtactcagccttgtttgtataagtccttttaaatattcttactagattttaacccgcggtataccgcggaacaatttattttttaaagataatatattaaaatttgtaaattttatttttataaaatatttatattttataatttataattgttattaagtaacgctatacgacgaatacatgagacaattgttaaaaaactgaagttttaatccttattaaaacaacatattaataatattttaaaattttataaatattgattcaaatacatctaccatataacccaattccaaaataaaatccgttctgtaatttctttacccgtctcgtgattttttttaaagtagtaatttttaaaatttaagaattatttattatatataaaatttttgcaaattgtatcattctctatatagatttatattttatagtttaattttatatatagtaacattatatataccacataacatttttggttttatataatcttttctaaattaggatgatttgatatgttaataaattaaagatttaacccgtattgaaacaatggattaattatattttactgtccctccaaatttgtcttgccaaaaagccatttattttattagtattaattttatttatgatatgactttgatttataatctaaacattttagctaataacataggagtgcactatatttatttaataggggaatgtaccatatgatccgaatgcacttttacccaaatccaccaaaaaagtcttgcaaaaatactatagagataagaaacgatagtcggttttgataggtataagattaacaaatatattagttagcttaaattagttaaaaaatacaaattgaaaaggtatattacactttatttgaatatagtataaaaacttatataacacagagatttgatcatttttttaatgttgaatgcttttttttgtgctaatttttatgtgattaagatttaattgatttcgtatataaaatattaaattgttttacgaacttatttgtttataatttgataacaaatagatatttgaatattcggtatattttgcttcagtttaagaaatctttgatgatccgtttttagatccaataagctctatatttaagaaatgaatattgtagtttaatgattttggtaatcttaatgacgtcattaaaggagtttttttttgattaaatttttctaatggcattgtcatgtaattaattacaaaaaataaggttacatttaaaatgtactttccaaataatatagtaggatatatacttgtctactttcacaagaaactacaccaaaaaaaggtattgttgattgctatctagatttcatttataatgtctttaaaacttttgacgttcctctttctcctatctgctgctgtgatctaccacattcccgtcgtgacttgcagaagaacatggtgcacggcgatgcctacttccacaactgagcagttacaatccaacattaactttgcttgcaatcatgtggattgtgcaccgatccaacccggtggattttgctattaccctaatactttactagaccatgctgcgtttgccatgaccagatactacaggagtcaaggtcacacatatgctgcttgtagcttcggcaatactggttacattatatcttccgacccaagtgttggcacatgtatcttttaactttagctttaaaaagaactcttcatagaacttataaaatgttattttgctttaataacgggtttctgttttcgaactcttgtatccccaaactaaaattattgaaatggacgttattatataaattatgcggaagttataatgttgacgacttttattttcttttactattgttgatttgttgtagttcaagactcaagttgtcttttgactcgttatagaatctaaaattggagacttcaatatatatttgttacaaatagtgccgtatgtaagtcttgtcaccaatcctaattacttacccaaagcctttgtagttaatcgttactaaggtgtatagagtatagtgcttgtgttaatattattttcacctgacttgttaataatttcaaaataaacttccaattgattcacgaaactatggagaagagttttctatatgtctacacaaatcatcatttatgtagtaaattcaactcatcacgttaactcttttagatgtaacgtaggagatgttgtctaatggtttgtacttagtggattcgggctccaaacaaaatttctgacaaaggaaggtacgcatgcatgagaagtaggtaaagaggaaattatcaaaagatagaagtggtggctgaagaaaaaaaatagtagaacgaaatgaaacaactgccaagattaccgcattcggattttttttggccacatacacaaacacttgtctttttttttttatcaagagaaaatcctttctttaaaagagaaaataatcaccgttcttgattattcattaaattctattagattttgtgtcaatggtgatactattatttgaggaattgttatggaaaagttattgaattgtttgtttttatcatatcatttatcaaacgaaaacttgaggaaaacttaggaatgtttgtcacatcatttagttattaatattttttaaaaaattcaatgttgttatcattgttatgtttttagtaaattatgaatactttagtctattgtaaatataattaaattttcattagtttaattttaaactagttatgttgaaaatcaaaaagattaagtagaacctctgagcgtgtttattcatttttggaagtctttagagatctactgtatgtatcttattgaacaaatatcaaaataaatgactagtaaattataaggaaatgtagaaaaatatagttaatagattttaaaatataactaattctattacagatagcatattatagatatgtgtagtgtttccatatatagcaagttagcatgatataggaaaagatcttaccaaaatttcaaagctactagataagatcatattagctaaaataaaataagaaatatagcataatttatttaatggaatgatttataaagagtgatgtgtaagtgttttattgatttattacatatcttataattccatctccttagatctcttcccaaagaaacaaaagcatatagagttatgacacttggtctactcagccttgtttttggagaaatctttttgcatattatattatatatacttaagaagtttcataagaaactacaccaaaaaacaaaagtattcttgatagctatctagatctcatatacaatgtctttacaatttttgacgctcatctttctcctttctgctgctgtgatctatcacatccccgtcgtgacttgcgaaccatggtgctcggcgatgccttcttccacacctgagcagttacaagccaacattcagcttgcttgcagtcgtgtggattgtacaccgatccaacccggtggattttgctattaccctaatactttactagaccatgcttcgtttgtcatgaactcttactacaagagtcaaggtcgcacttatgctgcttgtagcttcggcaatactggttaccttatttattccgacccaagtactggcacatgtgaattttaaatttaatcgaaaaaaacatagaaatcttcagcatacttatacatttttattttgttttaataatgggtttctgtttcggactcttgtatttccaaactaaaataaatgaaatagacgttatttaataatttattctgaagtttcaatgttcttagtgatgacgtcttttactttctagtactattgttgtggttcaagatcaggttgttttttgactcgttatagtattcaaaattggagacttcaaaaacttagatttgttacaaatagtgccttatatatgtaagtcttgtcaccaattataattacttacaaaaagcctttggagttaatcgttactaaggtgtagagtgcttgtgttaatattatcttacacctgacttgttaataatttcaaaataaacttccaactgattcacgaagctatatatggagatgagtttttttttttttcttatgtatctacacaaatcatcatttatctagtaaattcaactcatcatgtaaactcttttagatgtaacgtaggagatgttgtttaatggtttgtacgtactttagtggattcgggttacaaacaaattttctgacaaaggaaagtacacgcatgagatgtattcaaagaggggatgatcaaaagatatataagtgatggttgaagaaaaagatagtataacgaaatgaaccaactgcctagatttgtaaccgcatgcaaaaaaaaaaagagtcattgatttgtatgtttacatacacaaacatagaaaatgatcagagttcttaattattcattaaattctattagatttcgtgtctaaatggggatacttaacttgaggaattgctcccacaaaaaaaacaaaaaaacttgaggaattgttgaggaaatcttaagatattattttttgtcatatcatttaccaaatgaaaacttcagaaatattagccacatcatttagttattaatatttttaatttttttaaatggtattattattgttatgtttttagtaaattacgaatacttgaatattgtaaatcaaaattttcattaaattttttattagtttgggagatatatacttataaaacctttggccttatcaacataaacttttgaccaaataaaagtttaaaatattccagttcaattaactttgcccaaaaataagtttaaatgatctaagagttggaaaaaaattggaagtaaaaaacttattaatggtttgctaaaatagtttttaccaaagcgagattaaaaggttaaactcgatctcctacgtttgctaaaactcagcccaataggcccgtacactaattcggtttgggtccaatgataaaatcaatatttcttaaaccataaattgatctaatttggtttaatttagacttaaacaaatctttaacaaatcaaaccgaaactaaacccaaaccgaaactaaacccaaaccgaaactaaacccaaaccggattaaatgggtcaaagaaaactcagcccaataggcccaacagaacctgaaacgtcatcttcactctcacgagtaaagaaatttgctaaactgattgattctgtaatttcagcgagtcgtctcgtctagctctcacttttgttcgtctcttctgcgtcaaacatttccaccattttcgtttcttaaacctttcttcagctcttctcttcgtcaatcgtgggttctttcatcatctctgtaaattttctgggttacaatactttagggttttttcttgactcacatttccgataaaccctagttttttttttcagtgcgatagtggaattgaagtctcttcggtgagtttctttactaatggagaccgccgcttcgagcgtagctacaactcgtggcggttcccttcagaatccgtcgccgacggctccgtcaagaaaagagtggcgcgccgtttcggattcacaagacaccacagattatgtggtaacgtttattcttctgggttatgtttatgttcagaggaatgtgtagactgaatctgaaagttttgatttttcttgctactgttgcatcttttagtttgcatttcgtagtgttctcgttatagctcttttgtttttgtagatttcactgtaattgtttggtgtagagagtaaagtgatttgcttttggttgtttcctagttcatgtgttgactggcttagtcaaatttgttgcaggatttggaacagttaaagctgaacaggacagatgagagaacgatttatgaggttaatttcttctttcttgcaatgttgttacttagtttttgtacttattggagagtggggacgacttcttaatggggtttgttgttgtgcagaatggaagagaacaggatggttattcaaatagtgagatgttgcagcaacagattcttaatgtttctagaaagaaaggagaattacagcagttggagattgagcttcgagctcagatgattgcgagacatgagatcatggagatccagagcaactatgaatctcaattcacagagtatgctaatgctgctgctagaatgcaggttaggtttcatcctcctggtatattagcaggttttttatttatttatttcatcgagcattgtgttgcgtggaagttaaaggactgttgctgactttttaggagcaacttcatgagaatgagagatctattcgggaggcagagaggaagttagaagagaaagacagagagctacatgcgattaaactagataatgaagcggtctgtttgtttattcattactactagatttttttgtgatataaatttgaatgatttgttactctccttgttctgctactaggcttgggccaaagagggtatcctaagagaacaaaataaagaacttgccacattcaggtgctgttaattccttatctcttttctctttgtttagtctgtacgagtttacctattatactgcattaatctattattcttctatcttatattgggttctgctactttcgtttttgacaaatattatatcaggcctttgatttctacttgtataagacgtttcaggcatgaggttttagaaaatataacaggatacattttctctttctttaatatgcagaagagagcgtgatcactctgaagctgagaggtcccagaatatacataaaatatctgaacttcaggagcatattcaagagaaagagagtcagctcagtgaattgcaggaacaagttagttttcccttgtttttgtgacacgatcaaatttttttaaacatctcatgcttatggatctttcagttatatatcattgtcatttttgaaattctaatacactaaaacaagagtcgttaattttaatggcttgtagaataggattgctcaagaaactatcctgtacaaggatgagcaactaagagaagcacaaggttggattgctcgtgcccaagagattgatgctttacaatcatctacaaatcattcgttgcaggctgaattgcgagaacgtactgagcagtataaccagctctggcatggttgtcagaggcaggtcagttgccctgagtatcttccgattcctaattgattttccatgagacgcttctactctagtcttatgttgttccttgttattgagttcagtttgcagagatggagagattgcatgtgcacacagtgcaacagcttcagcaagagcttgctaatgtaagggaagctggtggttccaaaacaaactccggtggagcctcccagactattcagaacagcgggaaccaatttgatgctcatggaaacagcgcagaaagtgcaaacattagtgtccattcaaatggaaaaagcgcagataacatctcatcttttacttcaactgatgataaggctacccaggtacatgtttgtttctgtggattctcttgaatgatttccctctaattggcgttttgtgttggatttacaagatgatagcttatcacaatacaatgttagcttgtggtgagatgaatctgtacatttgctaatgcagttggcttatcagccgatttaattgttaaacattagatattgaaactgtcaagcaaaagtgaaataattggatattcccagcactccaaaaagtactgtgttgaaaattaagaaggccagtataaaagttctttgattttgtatttggaagtagttgtttcatcacttgtctattcaatctagtgctgattgttcgaaattctagatttgtcatgctccacttcatatcctaactgtaactacctttttcccgtaactatttgtagaacaaccgtgttgatgggatatcagcttccaatcttgcgacacatgggttccttcaagctggtcagatgacccctctgcattcatttgtcatgcatcaacaagagatttctcagcttgttcagcctcaggtcccttcacaacatattgaacaatcagtgttgctacagcaaaaggtgtgagcttaagaatgcagcttgtgtttcattttttatagttaatgaccattgtctttcattctctttttgtaggctcaggctgtaccagatagttcacagatgcctatgcagaatcatgtgcatccatctcaaggtgttcatggcttggtacaatcctttggtcaagggtacggagatattcaaactagccaagtagcacaatacggaactacgacaacaccatcctctgtgaatgaacaggtaggaggtgttcttattacttaatggggatatacgtggtctatacgataccttcttagaaaatacttcttaacaggcagtggaatctggcaatggagattataatggatctaatcagtcagagaataactttcaagacatttcttcacagttccgtgatgccctaaggcttgattctcattcccagaatcagaaacctgaggtatgcagtgcacatattacctagcaatttttcttggcttgtagagccttagttaagttggatatagagaaactgttctgaatgtgtgtgttttcaatttctatacccaggaactcaatggtcaggtttcacctgatgaacacagtggtgctaaatccattgtccctgaaactctagtctcatccggaaaacctgagaggaactcagagtgcgctctgctcgatgaaaggtcgcttttgacatgcatcctccgtactataccagctggtgggagaatcagaatcagttcaacggtgagcaaactaatagaaaaccacaatgagacttaaccattgaatttggaaaaagggctttcgtataatgtctgtttcatattgtgcaatacagcttccaaaccgtttgggcaaaatgctagcccctctacactggcatgattacaggaaaaagtacgggaagctggatgattttgttgctagccatcttgaggtctgtctcatcccaatttccaaactatttcatgtgtttctcaaaatattgaaatgctaaactggttttggcttgcagttattttcgatagaggatgactacattcaagttagagacggtgcacagaaaatggtagcagcatcagcagcagcagccaaagtggcagcagcagctgcagcttcgtcatccccaaactccatctatgtggctatgactcctatggctcagtctcaagggttaaagaagaatgacaacagagttaggcagagctctgattttatggtgaagcagcaaagaaagctctgattttacaagtcaagcttttcaaagtaaaagcgtattagagtgaggctaaacaatctcctatcttgtttttcattttttggtgtatccccgaagctaagatatacattcataattacttttgccctttcaatagtatcaatgcttaaagtcgacaatgtttattttaacatgcttagagctggccataaaaattcagtaaagaaccagcaaaactcaagcgcctgtcatcatttacacttacatttcttgcctcctacgtcacctaattttaactaatctaaaccctaatcaaatttaagaacaagtcaattaaaaatcgaagggtgcagatgaattgcgagaaaccctagtcaaatttctcatatgaacagatatcgactagtagtgtttacagaaacaaactacaagatacggagacatataaattgcgacaaactctacacaaaacacatgggagggcttgttaggtttagggttttcaatgtttctggaagtgactggaagaaatcaaggaatagtatacaaattgaagaagaagaatggaatataattacctaggaagatcaaacctattcgtcttgccttgagaccaactgtgattaaatatgattagggtttagattttatagttaaacggagtaaatattggtttaggtttataaacagatttatattttacaattaaacgaagttaaatatcggtttgggtttagaaaatacgattagggtttagccaaacttatttaattcacatatttagcacacaaaaaaaatgcaatggtataccaaaactgatacatctctctaggataaaacaatagtttttatctcaagggaaaaattgaattatttattttctaattaattagacaaatactgaattgtagtactagaattgacatataagacaaccttcacacgttttctcttttaccaatcaaatgaagacgacttgacacttggtagagcgtcttggtattgtgaagagtttcttgaagatgtcatgatctagagagagtttcttgaggatagccgaattctctagaaagtaccttactagcttcattacttcaagatgtccccggaacggtattcgaatatcaacaaactcgagagacgatagcaaacactcaggtaccaatgaagaaccgaattgaatcatctcctccgaaagcatttcatcattatcaccaacacatacctgaagattaaaacacaatgaagaacttcatatacagatggaaaatcttattgagtttagtagatcattaccaagatgagagatttcaagtttggacagctctcaagaaatgttggtaaccatttcaaatcatatatacaaagagtaacatgcaggcgtgacatgtaaccaaaccgaggcagcgattctagttctgagtatttacatatgagctgtggaaccaaaaatacatatatgatgtgggtcagaaacagaaactttcatgatagcaatgtcaaatactagctagaattgaaatatacataccttgaaagtgtccacccaaattgtcatatcgctgactttcaaaataccgcggagaaaactgtggatgctatctgtcctcgatgaaacacttgcttcatcaaaatctagcaaaccaaaggggagagaaatatctaatttggcattggactccaaattgtttactatatagcgttctgatacgttatcattgattttcaaataacttagtagaggagcatcaatcacaacttctgaatgaaaatcacacatagatttaagaatgtttatactcagcttcttcagtgatctagagagcaaccgtaaggttattgcattctcattcccacatccataaatctttaactcttccaagacaggacagcaagatacaaatctctcaagatttgcctcattgggaaaccaaacatattctaaatggatagtcttcagacaaggtaaggaaacaaactcggcatcatccaacatcaccctacagagttttaagtagactagtgtctcacatatgtaaaggcttaggggcatctcataaatatatttagctggaagacactgaacatgtagatgttggatcttacacttaactgcagcatcaatccatgacgtgagataagaggggtcatctacaccaacgtcataaccataaatatataacttgaacttgtttatgcatgaaaccctattggaatcgaaaaacttgtcaccaaaactcacaaaggcattgaaatctgggaagtataaggaattcagttccaaacaaggaagccaaagccagagactcctccatctggtggataacacgcgtgtcgtaacaaggtcctttatgggaagatgagaaagtatgtgacagatcaaaggatcaggtaattggcttatcatatctgccttcgatccctgattcaactctctaccaaccagttgttttctttccaagaatctattcggcaaaagaaaaaaaaacaaactgaaaacgcaagtcttaaagaaacaatttcgaggcgtagatattacgaacacatagcttgcgacctacgtaatcagcattatcgactaaaaccccaagaataacgagacgaacctaagtgcgagcgaaagccaattcgttaggttaaagattaaagattgaattggtaaccttgttgcaatcgagtcgtgtgtgtgaagacgtgtgtctcaccgtaagatttgaaacttcagactgatttcgtaacccacaagtactcgtgactcgtgagggtggagaaaggagatttctgtagataaaatttaggataaatctatctgactttaaatttagtttgggattaggtttcgtttttcacttaagcgttaacaacgtcggatcgagtttaatctcggtaaaaatttattttatcaaaactaaatttcctattaaaattcttcttcgaaagtaagttttaacttttatgtaactcgatttgaccttcctaagagacatcaaacgcaccataaagatcatcaattgagaatttgtaggagaccaagtccaatccaggaaccgattgccaaagatatctccatcttgtggacaaaaggcttgttcttacagcatcgtcagtagaaagatgagaaagtatttcagatacaaaatatttcattcgtttatagtccaagaggacccaaaatgatataacttaagaaatggtttacactacacctacttgcttcatatattgatgactgatcaactgctaagtaaagaaagcgtcacagggtgtttctagctagtcaaagacgatcccgacttgacatgttttagagattcttgggaattttcggagtttcttgacgatttcatcttttgctgaacaatactgcaaacgtagagctagacgtagagtgagtttcttcagggttgtggaattctctaggaagtactttactaacttcatccttgcaacatatactaagatgctgcttttgatatcgacaaactctagtgacgatagcaaacacttcggtacagatgaaaaattgatatgatagttcccattcaaaagcatcctcttagagttactattagattcctgaggataacaaagagagagacatataataaactcagtaaaaaatgataagcaaaactatatagctcgatagggttaaccaaaagcattaccatgatgagtgatttcaagttcgggcagctctcgagaaaggttggtaaaaattgcaaatcagatactccaagagtaacactcaagcgggacatgtaagaaaatttaggcaggtgagggagcggtattaacgccaagtatcggtacatgagctggaaacaaacagaaaagaatgtgattagtgatagaaacatgtccaagatctctgcattgacatataagataggtgtataccatgaaagctggtgaatgtattgtcatttccataacacttaaaatccctggaagaaatctgtcgatgctacttctcattgatgtaacgcttgtttcatcaaaatgccataaaccaaagaggatagaaagatctaacttgtcattagaacccaaattgtttactatgaagcttttcgatacattatcgttaatcctcaaagagcatagtagaggagcatcaatcacaactcccgaaagagcaacctcatggaggaaagaagagactcgttttaaactgaacctctttagtgactgagagtgcacccgaaagacttttctatccatcccataaatgataaccgttaagtcttccaagacagggcagcaagagataagtttcttgaaagtggattcattgggatacatattatcttctaaatgcatagtcttcatacaagggaaagagacaaacttggcatcgtatcttccttcaatctccgactaaaccctttggaacaatcctgtttcgatttctttctaccaatcatggtgttgatcattctctacgtagcgaaattgaaaaaataaatttcagagaaacaatcaaataaactatcaaacgaagacttgaagaagaagagatctcaccgtgattttggttcatagaggtcaatgaaggttactctttttttttaggggactatctccacaaccacgtagttttttctatgatagttaaggaattaacggcgtcgtttttgtgtagcttccctggtctcaactattgatagaaaacgatgtcgttttaatttgcaatcaatttgaaatttgtttttcttatcgtattcatttatttcgatagtctcttgggaatatgttattttactgtaaactttctagagtaaagatgagattgagagatctaaataaactttttaaaggggttttagatcttctgctcttataccatgacaagatatgaatatcgaatgaattggttgattattgatggacccttaggtctgtatttatacaagttaagaatcggtaaacctaaaccaaaaggaaatctaataaaagacaataatacatagtggaaagaagaaacccttactaaagtgagtctctttagtgattgagagtgcacctgaaagacttttctatccatcccataaatgataaccgttaaatcttccaagacagggcagcaagagataagttccttaaaagtggcttcattgggatacacgttatcttctaaatgcatagtcttcatacaagggaaagagaagatcctgcctttatccaaggacacctcaaagagtcttaaggaaaccagtgcctcacatttataaagattaaaatgaatcttatgaaactgaggcagaaactgaacatctagatgttggatcttacgcttagctgcagcatcaatccatgacttgagataaaagccatcttcttcattttcactaatagttaacttgagcttgtttatgcatagaaccctattggaatcgaaatacctatcacaaaaactcacaaaggtattgaaatcagagaaatctcgggaatccaattccaaactaggaaccaaaagccagagacttctccatcttgtggataaaacgcttgtcttaacaacatcctttgtaggaagataattaagtatttgacataacaaagattcatagaattggcttattctgtcttccttcaacctctgattcaacccttttgaacaagcctgttttgatttctttctcctcaccatgtctaattttttctctagataactttatctgcataactaaaattggaaaacacaagtttcagagagacagtttggttttgaattagatattcattcaagttcgacttaacctaacgtcccaatcaaaattctcagtctagtaatgtaaaagatttcaaatcaactaaggcttaaaacatacatggtgaccaaaagctataaaaagattcaggcttttggacctgtttctgatgggtaagaacaaaacgatatgtagaagaagaagaagaagaagaacttacggtgacttaggtaatacgaaagaggtcaatgaaggtttcttacatttttttttagaggaccagtacaatcataatgacgccgtttctagtaaataaacgccgtggtttcactgctctacgtaattggagtattgaaaaccacgtcgttttgatcagcggactagtctctcaagctattctaagaaacgaggtcgttttgattcgcggttggtctttctctctctctaaagcagaaaattcgtagaatcggagagatttgagaagagaacgaaggaagaagaagaagaggaacgatggggtacgtgtttagagtacgattggcttctttcttcgccggagctgcgactgcgtcttttatcggactctccgttctctacaaggattacaaagtcgctcacgaatcgatttctcagcaggtaatccatcgatttctaattttgtttctcttatcggatccatttctttcgattatctattgcgaacaagctctgtcaccgtagattttgttttcgagtagctgaatcgatacatttcgttagcagatgttgttaagaatggaattttgttgatctgtagaccatttctagggttctcgttgtcatctctgtgattctccgattgagattcatatgcaatcatggtggaagaaagtttcacacaattttgatgtttataataggaattagggtttctggtttgtgtttgccattttagctactggtgagattttgttctggagttagagtttcataagttccaccaatacgttaatgcttcattaacattactatgaattagttttattttgtgattcttgagatgccaaaattgctaatacgagaacaccttcaaacgtttggtattgatgaatgaatactgatagagttgatgcacatagatgtcatcattgtgaatatccatggttgccatttctctgtagctttgatggtaatgattatactacaattgcatgattaaaaatatgctttagaattagtttttttgagaaccttgagatgccaaagttttaacatgagaaaactgccaaacattttgttggttgaattgagtgttagctttgtgaagaaccgtagcagcattttctatgtagctttaaacgtataggatactgacattggcattgactgttagatttgatctctctttttctacaggcgaagtcttttcatgactctctggatagaaggatctctactcttgaaagcttgagacaaagtgaagctcctcagcttgctgagacaactgaatagtaatctgtctcgacattgaacactctctgcttggcaatgatgttttgagttgagatgctcttttttcggttttatgaataacttggaagttcttgtatgactctactgttataaaacaatgaagtttaaggtagtcgacaatggattgaaacttgctgtaacacttgccatgaacttgtgttcgatcaattattttggatctaacattagtagaatcttgttcttcagaaagatgaacttttgtctagaccatcttcaaagctagggatctagatacatctttctgaacagaagatgcaacagttatggtgaagataagcaaatgccattgactgtaccaagaacaatttgatatctcatgattaggtctaggccggcccaaggagcccagtgtcctaatatgggcttcttggtgggttggaagttcaatatctctaagactctcacgggtctggaattttatggttttgcatgtaaaattacaacttcaaaaataattgcaaaagatcaagatatatagtcatgctagtatcttactattgcaccatttagattcaatgtatgttattgacaagagaaattggcaaacaaacgaaaaaaacctagctattggcaaatgtaacaacaataacatgtttttcttgcaatgtcatttctcaattgaaaagatcttagagttaatatatctaccattcccgttatcactttaagaagaaaaataatctagtttcacataattaaagaaacacaataaatggggaaaagaaagaaggaatcataatggaaagacatgtcgagagcacatggtgtttgtatggggacagattccaatgtaagaaagaaaattaaataaagtggaaacaataatatgaagaaaaaaagagagtgatgcatttgtctccttggcggtggtatggtggcttcactatcacccgacacctcctcaaatttggaatacacctaatcaatcatgattatattacacacacatttcgttatgtagatacgacactcaatcacggatcattttgttgttccatcattttcctctttattagtataaacatcaaaaaacattttcgaaaacgtaaatgttggatgagactttgtgatcgcatccaaaccacttaattatttaaaattgtaacgatttttgttattttctacatatgagatttttttactatagatagataggttagtgtcacaaaggtagcaattggtttgatggatatttttacttctatagtttttgtcagactccaatgaacttactcatatgtcacttttaattggatgtgaaagaagatagagcatcaatattgaaatggcaatagactgagaagtcagaataattttttgaaaataaaggacaataagagagtgtccacatcaacttttaccatcttattttgtctggtggcttctgcttctttggatttaaaccttgagacaaatagaaagacgttcttcacttggctacctaacaacttcactttctgtgctcttgtcacaatcttaacccatttttccttacatctaacgtatatcattgattccttttgagaatttccatgttcaagacaatgagagaatatatttacgcaatttgatccaaacggtttaatttgtttattagtttgttacactttgtgtaaactatgatattaaggtaaggatgttggccttggatcagttcatgatgttttttagattaccaaaaacgttttagacttttgaccataactcttttgtaagacagaccatccctagtacactaatcaaccttgatttggccatgggatctacgagtcggtttgcggttaatatgtcaacaacgacaaaaacaactgatggatatgagatatgacggtttggtcatagctaggccggtccttggaaggctctttagggactgagccatgtcctgatttttactattacatagtaacattgccatattaaactctacatcaatccacaaactagattcatagaaaacatcccccaccgttagatcgttacatcccctaagtgatatgatattgatctacaagatgggtctgtcttttaaggattgatccaaatgtcggaagcatggaagcatagtataggacaatcttggcggcaacccatcactactccatatcctttgtatccacccaaatctctctccctctctctctatatatgttacattagtatgatatgaaagtaaaggcaagcaacgttttactcattatcttctcaccaaatttatagcccacaacggatcttttactttctttcttttgaaataagtcacatgttcttcagtctcttctcatgataatataaagggcaaaagaaaaatctcacaatctttacaagagtttttaaaatctctaaccttcatggactttctcaaagtttcagacaagacaacaattccatatagaagtgattctttgtttagtttgaatcagcaacaatacaaagagtcttcttttggattcagagacatggagattcatccgcatcctactccatgtaacaatctctctctctgttcttctcttcttttgatttgtttctcaataacgaaatgttattctcattgatttgtaacagatgcaggaaatggacttttgggttgttattactattaccctttcacaaacgcacaattgaaggagcttgagagacaagcaatgatctacaagtacatgatcgcatctattcctgttcctttcgatctacttgtttcttcaccatcctctgcctctccttgtaacaataaaaacatcgccggagatttagagccgggaagatgccggagaacagacggaaagaaatggagatgcgcgaaagaagtcgtctctaatcacaaatactgtgagaaacacttacacagaggtcgtcctcgttcaagaaagcatgtggaacctccttattctcgccctaacaacaatggtggttctgtgaaaaacagagatctcaaaaagcttcctcaaaagttatctagtagttccatcaaagacaaaacacttgagccaatggaggtttcatcatcaatctcaaactatagagactccaggtactgttctttgattagtctcaaaaccaaactctgtttttcttttctcataacaatactctgtttttggtctctgttttaatcgtgtttagtttttgtttcaacttgaaaacagaggaagtgagaaatttactgtattggcaacaacagagcaagagaacaagtatctgaatttcatagatgtatggtccgatggagtaagatcatctgaaaaacagagtacaacttcaacacctgtttcttcttccaatggcaatctctctctttactcgcttgatctctcaatgggaggaaacaacttaatgggccaagacgaaatgggcctgatacaaatgggcttaggtgtaatcgggtcgggtagtgaggatcatcacgggtatggtccttatggtgtgacttcttcactagaggagatgtcaagctggcttgctccgatgtctaccacacctggtggaccattagcggagatactgaggccgagtacgaatttggcgatctctggtgatatcgaatcgtatagcttgatggagactcccactccaagctcgtccccgtctagagtgatgaagaagatgactagttcagtgtccgacgaaagcagccaggtttaggagttatggagtttggtaataacgactttttgtttgagttttggcctttcttcaatcttgtgcctgtaaactagtactgtaaacagtcactttgattaattatggagttatattatcaatgcttgtttagtttttatttaataagtgcatctccttttatgagaaacccacaaagtttttcaaacaaaaaaaaattaatattttattataatttgattatatgattaaaattttattttaattaaaatattaaaccaatagtgaaaagggacatgtaatcatgagttgtaaagagtatctcaaagtttctcactacactctctctctttcattttcattattttgttttttttattgcgagaaactcttatgaaaaactcttaatggagatggtctaatttatcaagttatttgaaggaaaaaaaaaaatgattaatcgaattagatagatagttgtattcccagtaaacaattatagttttttttcaaaaagggtgaaaatcttaaattccgtttagttttttctaattttgaaaatatgtctatcttaaatatttgttctttcttttacgaagcgtcacttgttttagtttatatatatgataatggagtgatgagggaaatgtatccaatttagactttcagtaaaaaataaaacgctgtggtacacttctcaaccatgaatccaggttaagtagctacgttctcgatcttgctattattagagtttgatgatttcgtgatgcttcatcttcttctctattttcagaaatatgtagaattgtagatc

SEQ ID NO: 8 - protein, 675 aa, Arabidopsis sp.
MRSMMMEREGRNEIEREVIDDLEETQNEGDDFKSIPPWKEQITFRGIVASLIIGIIYSVIVMKLNLTTGLVPNLNVSAALLAFVFLRSWTKLLTKAGIVTKPFTKQENTVVQTCAVACYSIAVGGGFGSYLLGLNRITYEQSGGTHTDGNYPEGTKEPGIGWMTAFLFFTCFVGLLALVPLRKIMIIDYKLTYPSGTATAVLINGFHTPKGNKMARKQVFGFVKYFSFSFIWAFFQWFFSGGTECGFIQFPTFGLEALKNTFYFDFSMTYVGAGMICPHIVNISLLFGAVLSWGIMWPLIKGLKGDWFPSTLPENSMKSLNGYKVFISISLILGDGLYQFIKILFKTGINMYVKLNNRNSGKSNSEKDKQSIADLKRDEIFVRDSIPLWVAAVGYAAFSVVSIIAIPIMFPELKWYFIVVAYMLAPSLGFSNAYGAGLTDMNMAYNYGKVALFILAAMAGKQNGVVAGLVGCGLIKSIVSISSDLMHDFKTGHLTLTSPRSMLVSQAIGTAIGCVVAPLTFFLFYKAFDVGNQEGEYKAPYALVYRNMAILGVEGFSALPQHCLQLCYGFFAFAVAANLVRDRLPDKIGNWVPLPMAMAVPFLVGGYFAIDMCVGSLIVFAWNMRDRVKAGLMVPAVASGLICGDGLWILPSSVLALAGVRPPICMGFMPSKYSS

SEQ ID NO: 9 - DNA, 83698 bp, Zea mays
gatctctcatcttgttcataatacttttttctcctctaaccaactagacagtctatgaaactcaaataatataatttttaattttcggtcaaaactgtaaaaacatgtcaaaatcaaataatattttttttttgccaaatcagaaaattacatttttccgccaatgtaaaaataatttttagagtgacattgtcagtataaatcctaatttatgttttaatattatagctaaagtctataattatttttggctgcttcttgcatattattattctaagtttggtttttgtttttatctgagaaatttctactatataatgaatatttgaaactgtagtctatatatttagatttaatattttatacaacaagttgatatggccgagttggtctaaggcgccagattaaggttctggtccgaaagggcgtgggttcaaatcccactgtcaacattcacttttttctctttttccattatttacttgctgacgtgtactcaccttatcttacattatttgatgacgtgtaattttagtggtcactaatcgagagagtgagacgaaacgataaggagaagaacttcttcttcaacctcagaaaaccctaatttctccatttcgaaactccaacgaattctctggtgattttaccaatggcgatggcaacatcgatgagtttgaatctaattggggcattcaaaggcttgtctctttcttcgacttcgtcttttcttagaggcgatttgagtttttcccccaaaacctctttcacggtgactcttcctctggaaaatcttcaagctccgattccgttgacaattgaatcggcccataagaaaggagctggtagcaccaagaacggtcgtgattctcctgggcaacgactcggcgtcaagatctacggtgaccaagttgctaaacctggtgctatcattgttcgtcaacgtggcactaaggtaacacttaagaacacagaattgttgaaattgagaacctcttaagctcctctttcaattctcttctaatttcaccgcaactctgtcaatttggtcagtttcttatgggtttagctctcaagctctgttttcagattccattctcgaacacatctgggtttaggatgaattttcgtatttagtagctcttgtttctgatgccattgccatttctctggaagttctacgttatgtttgtgtttaggatggaactatattgtttgagaagattgattcatgaatggtgatgtttggtgcagttccatgctgggaaaaatgttgggattggtaaagatcataccatcttctctttaatcgatggattagtcaagttcgagaagtttggtcctgacaggaagaaggtatacctgtagttatactgagtaaggtttataagctttcttaatcttctaggatgttaaaggaaatgagacttgtttttgcagataagtgtgtatccaagagaaattgtaccagagaatcccaatagctacagagcaagaaagagagaaaacttcagattgcaaagggagaagaagaaggcgagacgcgagaattactcgtacacacttcctacaccagaacttgttcttgcatctgcctcagtcgatgatgctgaagccaatccggagtgctagaagattgaatgtggagtttccttgtctctttttgtttagtcttgtttatgaaaaatgatcctttcagagtattaagagttggttgtattgtcgaatccaagcctctcttttgtcaaattgagtcccaagcaggcggatttttacccaacttgagttattagtgtaatcaaatggttattgttaattagtcaaagcctcaaaggttttaatttagtgaatttctcttccaacttagtaattctgaatcattgataagagagaccaccaaacctttgaatgccccaattagatttagtcaagtataaatctagttcaagtgtctatttgaggtttttttattataaaaattataaaacttatttggatcccaaaaatggatccattgttagtctaagaagccgaagactttatgggccttctagcgaaataatgggttttagcccaattacttagcatttcggaatatgtagatagtggtacaaaaaagaaattgaataatattcattttcgtctatcattgcataattagtggaatgaaattattattctatgttttttttttttcatttatctatatcttgctcatcttgaatccatcaatttcgctaacgttttacctgtaccatattagatcctttgtgttatctttaggccatatttcaccagcttgaggtcaaatacttatacacatgtatgtaacgatatatatatgtaagatgacgatatattattgaataacactaaaggcctagagcaaaaatagtaattgatgagaagttttaaattctaggagcagtaattaagaagaagaacaaagacaaatcctaaaacaaagcatatcagtagtaaaaccttgtagcacaaagcagagaggcacatacatgagtgtaataaatgagctatgcatagacatggacgttgagaacaacgagaaagaggataagaaagagaccatagagaacagtgtgaacgaaaatagaagctaccgtcgtcttcatgttcatgaaacccaccggagattctttccccggaaactgcagtatcaaccctggcgacagaaacgcgaacaaagccgaagctatcaacggtgcagcccaatcatgcatctcttcttacaagaacaagaacaaaagaaaaagaaaaagagaaatggagaaagaaagaagagagtgggaaataggtttggatgggtgaaaaaggagatgatggaattgtgagaatgagaatctgataaagtctagttctttacaattaaaggaagagagagattttttggaattttgctttaggtgaggtgtcttaatttaatcgtaattggctatcgaaggagagtttgaatttgaaacgattctagcttaagcatatcaaatgctcgattgtattagaataaacatggtttgagctgtatatctattctatgcggtttgagcggtttgagcggtttaaccaatttacgtggtgcagttttgcctttggaagaataactttcaataatataattgtactctgttttacaattaagcaatgtaaaaacagaagaaaaataaacaatgtatgtaatttgtagagaaacggttctatgtcacattcaaaatcctatgtgattcacatgaatagcgacatatctttcggtgatcaataaatgtacacgccacttttcgaaatacgtttggaggaattttgttattttattacttggaatgtattcttgattagtagaatttgtttgacttgagaagactaaattagattacaagtaacgggtccaccacttgctaagcgaccaatatattccgtaagaagagaacaatatttctccacttttttttcctactaaaaacaaaaattcaacttttttatatagttattgtacacttttcatatctccaaaatttagtatttataattttttttggtgactataaaatcaagacgatataaatcttagtaatttgtttgacaaatatattttttccatcccgaaatgctaatctttttactttctagtgggcaatatatacgagattatctcaaaattgtaacaaaaaataataatgttggaattaactcaaaataaatatgaagaaaataaaataagaattgggcttagctcagcttttcttctttgtttcaactttcaagtctctgtttttgactcttttactctctctcacgagtcacgccaccgctttatcgaaaaaaggcgtctgcttttacgttttctacttcacagcaatggcggattgggctccagttctcgtcggagttgtattattcgttatcctttctccgggacttcttttctcgttgcccggacatcaccatactctccaattcggcggcatgaaaaccaacggcaaagccatcgccgttcacacactcatcttcttcgctgcttacaccatcttgatcctcgctgtcaatctccacatcactaccggctaatttatgggccgaaataaaggtaacccttttgctgaatctgagctctctccctctcaggaatcttctgagatctggaactctgtttttagggatttgtatactttactctgttctctagtttgtgttgtgcattgagttaatactgaaaaatgtaataatcattaagcttgtattgatgtcattaagttataatgcaaaatttgatgtttgattccatctttgctattgaattgtgaaatacaaaatcacatatttttagttgtctatacaatatttaccttgaaaccatatcagcaaagaagcagagattagtgaaaaatgtgggaacaaaccaaatttgtaactaaatgaagatgctttatgttacgttattacatgctgaagctacgagagatcagaggatattgttatctcatttgaattgtggaaaacaaaattaccgattatttatttgtgtacaacctttttaaatcaccctgcttgagaccctatgagccaagaatcagagattaggaataaaaagaaaagaaaaaatagatgaaggcgaagttgagtccaaagaagctgtctttgttacactacatgatcagaaatggatgtgaatttgtatggcaatgatcaagatagtgagtatacagaagtaaataatggcgtggaccaaaatcgaaatcccgcttgtgctcatgttcccgaattccatcactcttgtccttgccggtagctgaaatagcaatcctggtgagagtaggatgaacagagccactgctacaatcacaggtccccagtctgcactcatatctcaacaaaatctgttttttgaaatgagtttttgtataacaggttcttgtgttttggtggcaggtgaaaatggaagatgaaactgagaatggagaagcttgctagtgttttatgtagagttatgggtttcttggtggctaaggagaggaattggtttagatgctaagctttgtgaagactacaaaagaaaggtattatattcttgaagtgggatttgctttttttggtctatgaactttctttattcttaattcttcaatcaattgcgtcttttaagtttctgttttctattttctacaactgctttacagatcgttcttggtatgtgttttctgctttcgtttttttgcttttgttaaagaagtgctcaaaagtagatgactgtgtaaaaagatgatattaccagttgttgtttcgttgtaagggtctactttgatagttttcttcataggtttggatttagctttttcttggtcagtctcgaggaaactgttggacagatgaaagtcggtgtttaatattcttatgatgtaaacaaactgttcgggactaaccgggactgatttcggtaatttggtattgaagtttgtgtataggtcttgggtctaattatgttgtaatgcatagctaaagagagggaatacaaataaaagataatcatcacttcactttcttcttatttataagtttgtgtgtttgtatctatgtttcaactctctagccctttccaaattttaattctttttcctcgtttcaaatgtgatgtgttcaggttctgtagtagtagtagtagtttcttgactttaaacgctctgcatcagtgtgaatcttgaccaacttctcggtttccatggtcggggtcatataacgagaacttgttgtctcggtttttctccgcaagattcaaggttacttcaacttcattgatctttagttttgagttttgacaacagcagtgttgactacaacaagaattggtcaagattatttgtttctgggcttattttcgtactgggcctaaatttcaaatatggcccattatcctttgctcttcttctttaacacgaatcgtctctactcctaacttaaggatccacaacgttcttcctctttttctttatcaaaggaaagcaatcttcacaaaaacaaatatcagtttcggtgaccaagctaattttcacaccaatgaaagattttacttttcggcaagcacatacctaaacatcatttttttatgtgacaactaaaatattttttctttatctattcaattttttttcatcaataacatggatttgatcccatacgttaacatcatattagctatttcactaacacaaatcaccagcctacaagtagttttgcatgtatagttatcaacttatcaatcaatgatttttctagaatattactgttggttctgttgaataattttaaaatgtatgattattactaatttacctactccacaagaaacatgtaacgactgctctatggtcaattttgctcactagtctatcataaattacatatactattgtctcagtcaaaagtgacttgcgtgattctaattgaaaagtattgtgtattgaagttttgactagccaacttagaaagtttatgcatataacaaatttaaattttaaattaaaatccatttaaatttaatagagaattatgaattttctattaaaattttgaaatgtcaagaatttaagaaaatgacattctattgtaaataagagccaattctaaaatttatttaactttttttgcaaaaaaaatatatatagattgaaaaaagagtaatcattgattatctataaaaaaaaagaagtagtccaatttaattggaagtcaatatggtttagtccttttgctctgttcaaaaaaatcaatttatgttattttgttttgaaaaaccggatatttttatcttttggtctcaattctgaaatttggaatatatcagaaatcgctaaatgaaaacaatttttcatggaggttgaatactctaagtgggttttatatataacagttttcaaatttattgtcatatttacatctacggatctacctaatatgataagagcatgttttagatggtcaagttcaatattgttacatgccaaatatttaggagggtgtattcaatcaaacattttcaagtgatttgtgttaaaataacagatcttatgttattcaatcatgaatttttaaaagtctcataaaatctaatgttattgaactagtgattctaaaatctacttaaaagtcccctgttattgaaaacattttaaagattggatttgtagtgactttaagagacttaagtgaattttagtggatttccttagttaaaaatacagaattttaaatcccatggttttaggtagaatttgagaggattttacaataaatcataacaactttcttaaattcacccaaattctttaaaatcatataaaactcaaaataattcaaatcactcaaaatataattgcagttaatcctaactaacccaacaaggattttacagaattttttcgagttcgctgaattaataatttcaaaaacaaaatcagacattgttgaatcatttagtttcttcactagattcagtgaaattcagacaaataaaaacctgatctcaaaactcataaacaaaaacctagacctacgagtgaacaaacgaagcaaagcaggaattgttttgagggataaaatcgacgttacttatgtctggttttgtatgggttaaaacgcttttaagacaatctttcatgattaaatagtctaatatctgcaaagtcaaactagacggcattttccgaaaagtaaaattgaagggacgcaaataatatttatacttatttaggtattttattttatatccgaaaaagtaaaattgaagaaataaaaattgaattttggaaaataaaaattgaatttttgaaaataaaaataaataataaattaattgaaatatgagtgaaactcaaattatatgaagtctatgcaaatgaaagttaacttgaattattcttcatttaaatgatcaattatccttcatttaaactcaaatattaaaactggacttttaaatagtatctaacaattcacaaaaaatatctaggagatgtgaggataaaaagtttggcacattcttaaataatttaatataaatatgcaaactcaaatttaaaactcaacccaaaaactttttttaccactatcaaaattttgtagaaatatttaagaaatttgtatttgataatcagccaaactaaaaagataattgttgtgtgattacatgaacatggtacatcaatagaggagcaaaaccaaacacaaacaatctaaacaagatggactcctcaacacgaaacattaccaccaaatacatacaacttcgtcaaggaccttaaatttaagactactctatgccgaaacatagatagtagaaaacagagcaaggaacataatggcgagaggttagatagagtaaagtttaggtaaaatgaattttatatagctcctttacgtctttgtcaatttcatacttttatcaacaagacatatttccgacttcgattcaaagtcgtacattggaaaattatgctttagaaaaaacaaacttgacgaccaaaaatattttttaaaacttaaaaataaaaaaaaactgttaataaaaaaagtttcaaaaatagcttgagcaatgcatgaaagagagctgggagtgatagtagaagcaaaaaaaaacctttcaggagatatatataacccaaaggccaaagctctatattagaacacaaacattaaacattaggcatggaaaactctcaattagtttcgatcactttcttagcttacactataatcatatcaatcggctccatcaactgcatagacaacaataatcttgtaacaaaccaatccgctctgttcgtgtttggagattctgtgttcgatgctggaaataacaactacatcgatactctctctagtgtccggtctaattactggccgtatggtcaaacaacattcaaatcccctactggaagagtctctgacggacgtttgattcctgatttcatcggtaccaaaattactttttcgtttccttattttattttaaaacattttctttgtaactgatcgataacatggcagcggagtacgcatggttaccgttgatcccgccaaatttacaaccattcaacggtaacagccaatttgcctatggggtaaactttgcgtcaggtggcgccggagctttagtcggaaccttttctggattggtattcattagtaactactttacattggttttatttatttgttttcttttgttgaatccatggtaattgtttgtcttgtctatttttttttctttttcaaaaaacattttcaccactgcaaggtgataaatttgagaacacaattaaacaacttcaaaaaggttgaagaaatgttaaggtctaagttaggagatgctgaggggaagagggttatctcaagagctgtttatttgtttcatattggactcaacgactatcaatatccattcactacaaattcttccttatttcaatccatttccaacgagaaatacgtagattatgttgtcggtaacatgacagacgttttcaaggtaaaaaattttcaaaagggtttttgtagagtttctattgtgaacaacttttggtctgaaaaatgagagatgatgttgtgttcttaggaagtgtataatcttggaggaaggaagtttggaatcttgaacacgggaccgtacgattgtgcaccagcctcattggttatagaccaaacaaaaataagatcttgtttccagccggtcactgaactgatcaatatgcacaacgagaagcttctgaatggtttgagacggctaaatcatgaactatccggattcaaatacgcccttcacgactatcacacttccctatcggaaaggatgaacgatccttcaaaatatggtaacaaaacttggttaataattgcaatatatataacagagtctttactataaactttttgatcgttttgaagggttcaaggaggggaagaaagcatgttgcggaagcggaccattgagaggaatcaatacgtgtggaggccgaatgggactgtcgcaaagttacgagttatgtgaaaacgttacagactatttgttctttgatccttttcatttgacggaaaaggctaatcgacagatcgctgagctgatttggagcggacctaccaacattactggaccctataatctcaaagcgctgtttgaacttaattaaagctcactatctcgtcgtgtcatcttgtttggttttcattcaaaattttttgtgtatttgaattgtcaccagaatctttcagttttcttagactcttggtctccaaggctttacgcgagcgatatatatcactcgtttcgattttgagtttttgaccgtttgaactttgaagagtgtttcttcaaattaaaccaaaccaattttacaaaaccaaaattcataactgattcttttggttcacactaaaccgagtttatcaaaactactttaaaatatggtttggtttaaccaaattatacgtcttaatcaatgattaggagactttattaaagtctataaccgcaattcgtcatcaactaccatttccttctccaatggagttcttctctgagagtgagaggaatcactgaaaaaataatactgaccatttcccaaaaagttatgtacttattctgacgaaatctgcaggaaattctcaatttttttaaaagaagtagctttttggagttatggagacggagattcctaggtcaacggagatatcagagacattgttgttaccagaaacgaatctagatcacggtgaatatgtacccgagtggaaggaacagatcacgattcgaggcttgatctctagcgccttgttagggattttgttctgtatcattacccataagctcaatctaacaatcggaatcattccttcgcttaatgtagctgctggtcttctcggcttcttcttcatcaagtcgtggactgggttcttgtctaaattagggtttttatctaaaccctttactaagcaagagaacactgttattcagacttgcgttgtctcctgctacggccttgcttatagcggtaaaaatcgttacttgcttagatatgggttaatagtaatcttcttgatttggttacgtttagtttcaattcctatgtgttgatgatgaacaatgggagatatggatgatactaactttggatccacttgatgactttgctgaagggaactgtagctattccagatttatgatgatcaagtttgatttttttgtgtgcctaatctgatatagtttcttgtttttttaggaggatttggttcgtatttgatagctatggatgaaaggacatataagctcattggttctgattatcctggaaacaatcctgaagatgttataaatcctggattgtggtggatgactggctttttatttgtagttagcttcctcggtctcttctgtctcgttccacttcgcaaggtagcgtctcattactgcgatattagtattaatcaaaacaaaatatctaagagaacactattgtttgatctgttgatccttattcaatagtgtatcttaggtgatgattttggattacaagcttacgtatcccagtggaaccgctacagcaatgttgattaacagtttccacaacaatactggagctgagcttgcagggtaattgtcagagattcatcttcaatattcaactccctgcaaaacattgcttgagtttcattaaaaatttatctccaatattattcacatcagaaaacaagtcaaatgtcttgggaaatacctgagccttagcttagtttggagttgcttcaagtggttctttagtggtattggaggtgcatgtgggtttgatcactttcccacacttggtttgactctattcaagaacacgtaagttaaccaatttcgtacagtttaacaataagtaaatgagaatggtttagcatgtggatataattggtaacgagtcatgattttttctgctgtgcaggttttactttgatttcagtcctacttttattggatgtggcatgatatgtccccatttggtgaactgttcggttcttcttggtgctatcatctcttggggttttctctggccatttatatcacagcatgctggggactggtatccagctgaccttaaagccaacgatttcaaaggtctctatggatataaggtacacaataaaccgattaagtaggaagaatttaatgttagtagatgaatttcataggtactctcattgtggtggttactgattctctttcatataattactgtacttataggtatttatcgccatttctatcatccttggtgacggtctctacaacctcattaagatcattgttgtcactgtgaaggaaatctgcaacaaaagctccagacaacacaacctacctgtctttaccgacattttaggtgcgaatatatatgatcctgattcacaatcgcgtttgttagtaatagtcatagttattaataaaggtgtctctcatgaccatactgacaacgtctgtctcaaatattttcttaagagtttctttctccattcttaattcagataaaagtaagacctcagtattaatgcgggagaagaagaaaagagatatcatatttctcaaggaccgtatacccctcgagtttgcggtttctggttatgtgggtcttgcagctatttcaaccgctataatcccgttgatattcccaccattgaaatggtactttgtcctctgttcatacttagttgccccgggtcttgctttttgcaattcttatggagccgggctcacagatatgagcatgccttcaacctacggaaagactggtcttttcatcgttgcttcaattgtaggaaataatggtggagtcattgccggtttagcagcatgtggcattatgatgtcaatcgtctcaactgcagcggatctcatgcaggattttaaaacaggttacctcacactatcatctgcaaaatccatgtttgtaactcagcttttgggtacagcaatgggttgcataatcgctcctctcacgttctggctgttttggactgcttttgatattggagatcctgatggtctatacaaagcaccttacgcggtcatctaccgcgaaatggctattctcggggtcgaaggctttgccaaactgcccaaacactgtttggcactttgttgtggattcttcatcgctgctctgattgtaaatctaattagagacatgacaccaccaaagatctctaagttgataccacttccaatggcaatggctggtccattctacataggagcttacttcgccatcgacatgttcgtaggaaccgtgataatgctcgtatgggaacggatgaataagaaagacgcagatgattactcgggtgcagtagcttcaggtctgatctgcggagatggaatttggaccatcccatccgcaattctttctatattaagaatcaatccacccatttgtatgtactttagaccatcctagacattgttatcatgttaaaaatttgttgcattatggcagataatagagtttttatatctcactctagaacacaaagtgccaatagcaacaagtatgtggcataaagtcaaacagacaaggttgaccaacgaaactttatttgggcttattctgtaagcccatagacttttgttaagaagcccatacgaatctctttcctcgtcttttcgaccgaaattattaaaacctctaattcctcctcaaaaaccctaaaatcccaattgagaaaatttagacgctcaaggttttgtttacatcttgtccggtggcttgaaacaaattcaacttctgcgtactgtttctttgttgaatcatggatccagcgcccgaacccgttacttacgtctgcggaggtaagctcgacttgaaaacttcatcgaacatctaattttgctttgaaatcaaagttttgttagtatctgtaaaagattgtagctttgtgaaagtaaatgctaaagttgcctcttttgagtagccatatgaatcctaatctattaggaaatttcataatctggcttagttaggaggcataaatcaatcttgatcatcaatactttactgtattgtattgatctatttgacttgaggtctttttgaggaggagctttgcttctgctaagtatcaagcttctcccaagcagcctcagacatatacacttttaaaccaaaattcataagtaaaaaggttgtcttcgttttatgggtttgcttaatttcttattttctgtatcatcagattgtggtcaagagaacactttgaagtctggggatgtgatccaatgcagagagtgtgggtaccgtattctctacaagaagcgtacccgtagaggtgacttattcttaatcctccttttgttttgtaatgtttttagctttctttgattcctctttttttggatcttgttcctagtttataaaagtgatggcaccattttgaatgcgtctcaacatatacatgatatcgggatccttttttaagtcttgatcatagtttatgatactgatggaacgattttcagtgtgtcttatataacgcaaatgttctaaggatccggtttccgaatttatcttgagattgttaatctgttggttttcttgtacaaaatgtagatgttgtaaacttgtttccggctaatttatttgatggtttattctcttgcagttgttcaatacgaagctcgctgagaactactactcctgaaggtggttcatagtctgttaagatgggttgagactttttggcactgtttgtgatatatatatcgacatggtgattgctacattgccagttcgatttgatttggtcacagtgtgactgtgtaatgactttaccgttttgatagatgtaattcttggaataactaagttcacattgctataatccttgattggctataatctctcgttgttttgctacctttgttctttagtgtactctgcatttagttgatcagattctggtggtggaaaaggtagtgactaccatatatcaatcttgtttgctacatattaagtgttcggaagaaatcgaagagagacatgaagagaacccaccacaatgttactctcaaaatacctttttagtaccaagcagatggataaattagtgtgttaatatctgtctaacgaaacctttggtaagtcctataggctatggctacaaatagaagagtcaacacaagagaataatcgcggcaaatatgctctctattagctttcatcaaatacaaactatgagttttcaatctcatgatacatttatctcgaataccaaatgctatgaaataatctatacgttactaaatactagtactctcttcgggtggttacttcactatttcatatcattcattaccgaatttacaagccttaattgccattgctatcgtccttggtgactgtatctaaaacctggctaaactcattgttgtcgctgataaactcttccaacaaaagctccaaacaaaaccaactgtagcattttttttttttttttgtcaaaaccaactgtagcatttatataatagtctgggttatcaaaatcaaatacattctcagagtttctatctgctgacataagaaagtaccaacacatattatcaaacaataaaaagtaccgaaaaacatgtaaggtatggttccaaacttaatcttcaggatcatagggtctgttgtcaaaatcctctctgtcctcagctatgtcagggcaatacctgtcagacaaaatctcaacttgttctgcaaatgttttcgatttcggtacgccgatatgaatgatcatctggttccaccgttttctacaagcttctccatctcgattctccaaaagattatcccaatccacatcgtcgatacaagcagcatccagtttcaaaagctcttccaagagcctatagtcatcaacgttagcccacattccctttgctaccattggtgatatcaactgctcataccatttctggcagcaagtcacgtggtcccgtgtttcaagtacgtcgcttatagccatccaagggatgttatctttgagcataccatgttttgagtgtttctctttgaatgctttcattctaaggtctttgttgacaaggtcaaagaggttttgatactcctccctcatccaatgtcctttcttcttacctgccaatcttcctctcctccaagcgtctttcacatgcttcctgtgcttacccattgcatctgcaagtgttttccaatcattcccgtgtgtcttttgaaactccataacgagttcaatatcctctttcgtccatatcccctgagatcctgcttcaaatatagtatgtgcacgatgatacacactattataagtcctccaaggtaaagcagatgtaatttctttccaacagcctttaagttgtgggtatgccttgcattccataaccatctttatcccctcatctcctaaagcatggttatcaatgtattctaatacagcattctttaccatctcatcttcttcttttgtaaaccgcttacccctcaccaccttaacttcttcttcatcatcatcttccgttccttcatcatcatcactaggaaaaaactccacttggtcagaaaacttcacccttttacttgatgtagatggggtggtcaagtcttcatcacttttatcagacttttcttcagcctcagagacttcactttgcttcttcttcttcttcttcttccttctcttctttgcgtctttggtagagttcaagccgttttcttcagtctctgaatcctcactttgtttctttttcttccttttcttctttccatcatttgtagagtccaaattgatatcttcaacatctgaatccacacttggtttcttcttgttgttcttcctcttcacctccttgtctatgttttcttcagctcccaaatcagtatcatcccgctttctttttccctttttcttatcagatactttggagctttcagtattttctatcacatcaccaccagattctttcttgctcttcttcttcttcctcttcacaccatcatgagcttcagaatcaacctttgtctccttacttttctccttggatttcttctccacaccatcatcctcagcttcggaatcagcctttgtctccttacttctctttttcgatttcttcttcacagcatcaacagcttcagaatcaatccttattggcttcttcgacttcttcttcttcttaactataaaaacctcatcacctagtttctcactttcacgcccagttatctccatatcttctccagtaaaaccatccttattctctcgcttattcttcttcctcttcttttccttatccttcatagaactgtgctcggaaatgaattcttcaccggtttcttcagaatcaaccttagcgtcactctttttcttcttattcttcttctcctcagccatctcatataccggcggacacggttacgaaaccctaatcacaaaaaccgccgcgactttcgctcaaatctaaatccgaatttcgattgagtgattttaagacacagcgatgctgagattgattgtgaaggagcttttggatacggcggcttccttcatgaatttgagatgattagggtatttaattacttttgttaaaaaaaattcctttgaacttgggtcttcaatgacccactacccatatttggggcttgagtactactaggcccaacgtattgctcttgacgttatcaagttttaacaagagtttgatctaatttctatcacttatgttacaacttacaaagttggtggttatattgtgaaatttctaaatgatttggaagaaaaagaaatcaaataattactactagaataaatcagtatataagagaccttgggaaaagtatttaaaaatgtagaatcttcttggattaacttcaatgttttgtgtggtggagtttggatcttacatgatcatttgggacaaagctagttttcatcaaagggatgcaattgaagaacatactaaacatatataatcgaaaactgatataaaaaaacataaacacaaaggtttgttttgagttttgaccttattatcaagtgatgaaagaatgtaaaacttagtttttaatgatgttgataacctcttttgatttaaataaattctttttaatgtgtgatttatgctcaaaactaaatttttctaatgttttcatattatttaattttgcccttgttaactctcatggatgttgccatgttgatttgattgagctttggattggaatttttttttttatttttttttttttttttttttctttggattggaattcattcggtaagtaagaatatttcgtgattatagaacttctaattatgtaaaaaaaataaaataatgcgtgaaaataataaaaagttgttaccctttttttttacttaggtcaagaggtcacaacacacgccccaccttgaatctgagagagagagagaagtacgtccccacaccttgacatggtcatggagcccaagaagaaccaaaatctaccaagtttcttaaacccatcacgacagaatcaggacaacgacaagaagaggaaacaaacagaggttaaaggtttcgacattgtggtcggcgaaaagaggaagaagaaggagaatgaagaggaagaccaagaaattcagattctttatgagaaggagaagaagaaaccaaacaaagatcgtcaccttaaagttgaaggaagaggtcgtagagttaggttacctccactctgtgcagcaaggatttatcaattgactaaagaattaggtcacaaatcagatggtgagactcttgaatggttgcttcaacatgctgagccatcgatactctctgctactgtaaatggtatcaaacccactgagtctgttgtttctcaacctcctctcacggctgatttgatgatttgtcatagcgttgaagaagcttcaaggactcaaatggaggcaaatgggttgtggagaaatgaaacaggacagaccattggagggtttgatctgaattacggaattgggtttgatttcaatggtgttccagagattggttttggagataatcaaacgcctggacttgaattaaggctgtctcaagttggggttttgaatccacaggtttttcaacaaatgggtaaagaacagttcagggttcttcatcatcattcacatgaagatcagcagcagagtgcagaggaaaatggttcataaagctcaaacctttaaatggcttttaattagatatatttagcgagaatgtgattgctagtgaaatctcaaacttgatagcttattcatagcttattttacttcgatcagcaagagaacaatggttcctaaagctcaaaccttttttggcgtttgctttagattgtgcatacataaagtgagaagatgaaatatggttttgctgaaatctcaatgtgctcattcatagtttattttgctcgacctttctttaatcaatagctttgatgtactgattactttataaagttaagaagtaagttctgctctttaaactgtactgaatggaggcttctctgcataattaagcaaacactctgatgattgagtttcgttgtacaaaggaaagtttttgccttagttagaaattcatcttgtttctttgcccagaagctggcttgctgatagaatttggactacgctacgttttcttgggtgaaaaacaatccgcgtccttccacgagaacagtgaaactgtacgatgttcttattgtattgtgtcaacagcccataatcaatgaaatgggcctcaatgacagtataagccgaaaaatattcgaatgcgatctagaactaatctgaaaccaaacttctcaaatcattaacatttgattagggcctaaaagtccatccgtattttaaaaaaaagttgataacaaatgcaaaaatacaaatatacattactattaaaactcaatctttctgattatatgtggatggagaagctgttttgcagtgtggatttgaattttgaagttcttgttcaaaaaagaaaagaaacttggtcaagcttcaaaagaaagaaagaaataaagacaagtatacaaatacgttgacttcaattaatatgaagatagaagagaaagatatagaaatgcgttttaaattggcggtgggagatatgtgattcccttggtcccgctttctcgtttgattcggtaacctaaaccttctccatccaagaagacacaaacaaggaagaaattaacctacgtacgtacatcatttgaactacttatatctattataaatggaaaatctattttcttcttctctctcttttaaccgaaaaaggaaaagtcctcgaaataactctttaatataattcatgtggaaaccttctatatattttttgcatgcttttttgaaattaagtaatggcatactaattagaagaagacgaaaaataatatgtctaagtaattgagtttcgtggtcaaggcaaatcaagattgcattctttaattaagtgtacaaattatgatgtaagaaccgttatttggcataacctaatatatattaaaaactgattttttttttttttaacttctgtaagaaatggttttcttaattaccaatcatgatctattattgtaaaatgtgtaattttgtgttgcgattgaagtgttcttagataaagatatgtcatatgcatgatatcatctatttgattttgagaaaataaaaacataacatgagaacatcgtagagagtaagatacatgacaaagagaaagaataagctaagaagtcatccaaaaattgggaggcctactctcaactaccaaaaatacaagtgtttgttgtgttaggtcatttattcagtcacctaaccctaactatatgaaatacgttaaaattcatatatagaaaaataaagaagaaatactattagtcaatataattttttttaacaaaataaaaataaagaaagaaacactattagtcaatgaattaaaaaaaaaaaaatccatctttcgatcaagaccgtccaagtattctcctttcacataactcttacgttttacactaaacaagacgagatttttatgagcttgtggctcccacaacaatcctcattgatttctccatatatatcacatgagaatccaactcgacttcatactaacattcatcccaaaagttccattctttttcacaaagccattgtgctgttttctccatttggatcaaaatggtttgtgttatcattcttgtttttgtttcaactaagtgttcaaaattctgtttatttattaagctcacgcttcttttgtttttcaggttgctgagaacaataaaaacaaggacgtaacactctctgcatcaatggacaacaacaacaacaacatcaaaggaactaacattcatcttgaggttcatcaaaaggaaccagctttggtcaaacctgaatccgagacacgaaagggcctttacttcttgtcgaatcttgaccagaacatcgccgtgattgttcgtacaatctactgtttcaaatccgaggagagagggaacgaggaagcagtccaagtgatcaagaaagctctgagtcaagttcttgttcattactatcctctcgctggacgcctcaccatcagtcctgaaggtaactgactagttcgatttcggtctctatcttaatcaaactcagacaaaaacatcatctaatgtctatgatgtgggtgaattcaggtaaactcacagttgactgtaccgaagaaggagttgtgtttgtggaagcagaagcaaactgtaaaatggatgagattggtgacattaccaaacccgatcctgaaactctagggaagcttgtgtacgatgtcgtagacgccaagaacattcttgagatccctcctgttaccgctcaggtctacttttcttcctcctcaagcaatcgttttgtttcttagggttgtaagttttttgaagttgaagggtttgaaattgatttgaattaacaggtgactaaattcaaatgtggagggtttgttctcggactctgtatgaatcactgtatgttcgatggaattggagctatggagtttgttaactcatggggtcaagtcgctagaggcttaccattaacaactcccccattctcagacagaaccattctcaatgctcgaaaccctccaaagatcgagaatctccaccaagaatttgaagagatcgaagataaatccaacatcaactctctttacaccaaagagccaactctctacagatccttctgctttgatccagagaaaatcaagaaacttaagctccaagcaacagagaacagtgagtctctcctcggtaactcatgcacaagttttgaggctttatctgcttttgtctggagagcaagaaccaagtcgttgaagatgttgagtgatcagaaaacgaagcttctcttcgccgttgatggtagagccaagttcgagcctcaactgccaaaagggtacttcggaaatggaattgttctcacaaactccatctgtgaagctggggagcttatcgaaaaaccgttgagtttcgcggtgggattagtcagagaagccattaagatggtaactgatgggtacatgagatctgctattgattacttcgaagtcacgagagcaagaccttctctttcctcgactcttctgatcactacatggtcaagattgggtttccacacgacagatttcggttggggagaaccgattttatccggtccagtagctttgcctgagaaagaagtaactctgtttttgtctcatggagaacagaggagaagcatcaatgtgcttcttggacttcccgctacagccatggatgtgtttcaagaacagtttttacagatataatatgaacaacgacaaaatacaaatatctggcaattattgtcaaaacctattcgttttactctgtttttcttgcttccttttttttttatctttttgatttatgctgaattattattgttatcttcatcttaaacttgtctgtatcttgcatcaagtttcaaccataaatttatattatttcaaactatattgtaataatattattacttgaactgtttcagagatgttgatgtttttaacaaactaaaactcatatataaataaaataacaaaagtctaaagataaaatacagaggacaagaagacagaacagagtgatcctgttcttgtcttaaggacaaagttagagtagaaacaaacaaagacttagattattgcagtttgttctctattacttgccacacaaatgaagattcttctctaaaggtctgaaatttaaagcagtaaacccatagacacagagattagatatcttggacttaaacacaacctgctccgttgtagttctgaagccaggaacgttgcaggaatctttggacctaccgatcaagcttgcttgacaggaagccattagagaatctttctttgcttcagcggcgcaagcaacgccttccagagacgttatgtctagcttgtagagtccaagctcatttgtggttcgatttgcagagaacgtaatcatctctctagttctcgatgaagctgaattgaatctgcagattattctcacttccacacctgtatttttttgttcagttaaaacaaaagtgagagacattttaactataatttcaaacaatgttgataagattctttaccgggaatgaaatagctgtgattggagaaactgttgttggagcagacgtcgcagtaaacaagacccattacagtgatctttccatttggtttggctgaagaatgtttcagagacagagagtttaaggacagaagttgcagaagtaacagaagcattatcagtttctgcttcatgttcacaagtttcagtttttttctttatccttggataaacacttgtttacagatgaggtttcctttttgtttatgtggttgatgaaagaaacagacaaagaatgggccaagtggttaaagacaaaagaacaaacaaagatcaaacagtaactccattcaaacaaagcttcaagcttgtcttttttataaaactcaagactcttgtggaaaaagagatgagtaaagagaggcacaaagagatgagaggccaattaaaggaaaagtagagaaactgagagattggttggttaggtttaacatttattgagctctcatgctctctattttaagggtttttgtctatgaatccccttataaaaaatggctaaaactggaaaatggaagggagaaggtaagccactagtgtgtatatatatgtgtatggaccactgaaagtgaagtgtaaacatcatcaattcatcagcataaacaccaagaaagtagtcagaggatatggctctttaaatatcaatacaagaagccaaactatgatattttcccggtttactctcggttgtggctgaattctggaaagagaatttcaggttaactaactatacaaagataagctcacattatagacttttgttcgtcacattatagaacagcataagactttattgcataacaatttcaggtttaagtgtactagagaagataagcttacattacaagttcatcagttggaaacaatatatgggcttgagctttaaagaaggcccattaagcttgaaattgttcttcatgtaagattgctgcagattttactttaattttttttttctttttttcttttgcccttaaattcttgtaatgcttctaggtgactccatccactttatgtgttgtttcccaatcatttctattttcaactttattttttcctttcacaaattattttaagttttaacattattttcttcaacatacgaaatttatacagtagtttttttttgcaggtacatataaatcaatagttgagaaaaagtggaaaacaatagagataaaaaaaattagatgtaaattcgctgtaaaacaaaatgtttaaatttcttttccgttatcgaatctcttttatgaataacaaggtcgagtactcaaggtaaggaaacagaccattagaaaagtgccacgtcagcttatttcgttcttcacttttagcttctgaaaagtactttacctgaactaaaacgacggtgacagactgactccagtagctgctaacagtcgatttttaaaatattatatttatttgcaggaaaaaaaaaaagtgaaggaagaagaagaagttggagaagccaagagcaaatctaattctctgactactctcattttcatgtcggagacttgccgaaatctaaagaatttcttcaacggagactgaaaagctttctcttttcttctttcgttatcttcgttttagtgttttcccctttgcttacatttgaagaaatcgagagcaagcgatagaaagtgagagagagagatgtatgtggtgacgcctcctcaaagatccggttttggatccgattgtgatttacgggtttaccaaacatggaaaggaagcaatgtgagttttaatgtgaaagctttaggttttggaattacttgcttctgtgtttatacttgagctagttttctgttatttttcctattattgtgtataaattaattattaatccgttcatgggctcagtttactctgtttccaatcttgtaaagcttaaaaattgggacttgggtttgtgttagaagtgtagctttctgggtagttgtctacatttaatcatgaaatctttacctttaatcaataccttaggacattatctgaatatgcttgctacatttataggaatgcttcttcatacaaaggttatatagttgatgctgtctgtatattgctttcaatgtcgaatttaaatctggtttagtagtaacctagtggaattaaacattttgaggtttgttccttaaatccttctacccaaagtgttaacatattttctaaatttgtcccatttctgcagatattttgtcttcaaggaaggtttatcttcgggccggatgtaagatcacttggtctgaccataagtcttattgtggcacctgttacaattttctgcatcttcgtcgctagcaaactaatggatgacttctctgatagttggggagtctcaataatccttgttgctgttgtcttcaccatttatgtgagtacaaaagtttagctttgcattgtaaaacgttttgtgactggatcattcataacaattaatgcttccctcaggatttgattttgctgatgcttacatccggaagagatccagggattatcccaagaaattctcatcctccagaaccagaagttgttgatggcaacacgggctcgggaacaagccaaactccaagattgcctcgtgttaaagaagtggaagttaatggaaaagtattcaaggtcaagtactgtgatacttgcatgctctatagaccgcctcgctgctcacattgttcgatttgcaacaactgtgtagaaaggtttgaccatcactgtccttgggttggtcaatgtattgctcaagtaagtttctcggtttttcgttttcctgtccgttagtttctgacatcagtctatataattcatctcattctccttgggaatttgcagagaaactatcgattcttcttcatgtttgtcttctccacgactcttctctgtgtatatgtgttcgccttttgttgtgtctacataaagaagatcaaagagtccgaagatataagtatcttgaaagcaatgctcaaaactccagcttccattgctctgatactctacacattcatatctacattcttcgttggaggcttaacatgtttccatctctatctcatcagcacaaatcaggtaccctaatcatttgcctctttctgaaaaaacctctctcattcgatatgcgtgtttaatcgtttgatctttctttattctgtagactacatatgagaatttcagatacagttatgatcggcatagcaacccacataacaaaggagtggttgataacttcaaagaaatctttttttctccaatacctccttcaaagaacaacttcagggctatggtaccaagggaaaatccaatgccgtcgagatcagttgttggcggttttatgagtccaaacatgggaaaggccaatgatgatattgaaatggggaggaagggtgtttgggcaatggctgaacatggtgatggcaaagatggcaataacaatgaacggtttcatgttaacgacaatgagttaaacgagttatcaccagacatggggaacatagtgaatggtgatgagcaaatcaataggccaaacaatcacccgaggaacgcaaactgggaaatgtcaccagaagttatggccttatcagcaagaagagcttagaagcaggttaagagaaaattgaaaggtgttttaaatgtttgatgtattgatgtgttgtgtgtttacacgggaatcaattgtgaattggtgtgatattttcagatgttatgagtatttttagttgttcttcagacatttaaatcgtaaatcattgtgtgttagtgtaatctatgacttatctttcacagaatgagaaaacaaaatgaataccgacgattttcatactgtactttgcatatgataaaaattgcagaaactgaattttcagacaagtggaccagaagaccagacaactcttattatagtcttcattgaagcacggctctgtaataaatgcaaaagagctctaaaggcttcttatttgttcttcaaagctctaaagtctaaaggttagagatttaacatcacaagccaacatttttggtttaggaaattgaagattagcaataatataaagtttttttggcattgaacaaagacaaaacacaatgaacataatcaacaaattggtcaaactaaacattaataagtgcctcaaagtatcagactaaatcattttcactaacatttatgaaaaaataactgttattaactgaggacgaaggtaactgaccagtcaaacatcatagacaaagacaataaagagagtcaatagcattgttcataattgttatttgtatgcacatgttataaattatataaaaagactaaccttatttcaattgagatgtcactattactgttttagcatcctcttctactctttatttagaacctttagtaccttaccatgtgagtgaagagttcaaaaacctctgcaaaaactttcttttttatctctctctcttctttaaacttactttcaaacactaaatagccaaaacttaagataccaaatttcaacaggtcggctttttcgtttgtcttctcttaagactttaccaaaagagatttgtggtttctggagttgactttggttgattttggtgtatttacaggggaaaaaatgtataagaatcagcttcaagagcttgcacagagaagttgtttcaacttaccatcatatacttgcataagagaaggaccagatcatgctccaagattcaaagcttctgttaacttcaatggtgagatatttgagagtcccacttactgttctactctcagacaagctgagcatgcagctgctgaagtttcccttaatgtcctttcgtctagagtcccttcaaagtctctaactgctaagattcttgtaagttctctgtcttttcttcttggttttgatcatacacaaaaaaatgcaaaaagagtaacatcgtatatcttcatgtcactctttatacaaggatgagacagggatttacaagaacctgcttcaagagacagcacatagagctggtcttgatctaccaatgtacacaagtgtgagatcaggatcttgtcacttcccaggtttctcttgtactgttgaacttgctgggatgacgtttacaggagaatcagcaaagacaaagaaacaagctgagaaaaacgcagccattgcagcttggtcctctttgaagaaaagtatatataactatcttccaaattgttccatcaaccccaaaaaagcttctcacttttctacttgaaaccatttttctgtttctctactgtgcagtgtcaagcttggattcacaagatgaagagaaggaacaagaagcagtagcaagagtactctcaagatttaaacccaaagaagtaagaagacgagagacaacaaaccaatggagaagaagaacaagtcaacaagattcaaacaaggatttgttgattgagagattgagatggatcaatcttttgaccaatcaagcttcttcatcatcatcaacatcaacaccgaatcaacacaaaaactccagtttcatctctctaattcccccaccaccaccacctaaatcctccaaaattttgccatttatccagcaatacaaagacagatcatcacaagaagctaagacagagacagcgacagagatgatcaacagcaaagccaaagtcaatgaaacatcaaccagattgagtaagcaaatgcccttttccgacatgaacagatacaactttgtcggagggtgtagcgtaaacccttatagtctggctccagcggttcaaatgagatcagtgattccagttttcgcagctccaccaccgaaaccaaaccccaaccttaacccatcatcattatcatcatcagtcaatgaattcacctcatccaataattcctgctctgttctgaatacgccgggattgggaggccaagagaagaagaatctaactcgcgaaatgatcaaacttggttcagaatcgagaattttggatcaaacccatgatagttagctaaatttggttaatgggttaatctcaaattaaatccttgtcaagtgtttgtcacatagataaattatagaccagtatgtttccgtttcttgaatgtgttcttgttttttttgaagtgtttgaattttctaaaagtccgacatgaactttgaatgcttgcgaattgcacagagaacgaaaacaaagagagagcattaattagggaagaagatgaagaagagaaatgggagtttgcaagaaaacaattaatgggttttgttttgtcttcctccattaatgcgccgaatgctctgctctgcttcccactcttcatcattaatggttttgctctgtaacgtcggttccaacaaataaacttaataattcgaaattataaatttcacaagttacatttttctttgtgtaaagattttggaccatataaacattatggtttgcgagaatgacgtttgtgtcattatccgatttagatccgtataatctatgttgttaggaactcaggattttgctcaaatattcaaattggtatattttgactttgatgaaattattttggtcagaccacgagacttgattgcttctcaattatttgggccggtccattaatttggggccatctaacttttacgtgggccgtaacaaaccgaaacataaaactagtgacctacctaaccatattaccccaaccggacaggaggtaacgagttaaccagccacttgttcggcttggaaatttttttaatcgtctgacaatttatgacgtggatccaagtagcaagaaagctaacatgttgttggatctgatctgatccacgaacctaccataaatcaattttctcattatcttgacaggtatatcttgcattattatacgtcagattttcgtggatacaatatttttaaaatgcaatccatatttacttgatttccaaaaatgggttttttcaaaagcacaatttccaaaaatggatcacaatttattaacggattctcaacttgcaaatcaagcttaaattataatcttttttaactcagaatttaatataatctttttttactcagaattgattaaactaacagttatattgatcttgaaatcacctttaatatcttgttttaaatcttgtgtcttgcaaatcacggttcataaatattggagaatcgaataaataaattatttctattcctatattgcgtaattttattttttgactctctattgcgtaattacactcgtcagatttcgtggatgagtactcagtaaatcttattttaattaaaaagcttactagtctaattgctgcaaatttgaccaactatacagttgtgttgcattacaatatttcaaaccattaataaaacgtgtaaaacatatttggagttgaatgaaactacattttcctttatgtcaaacgcacttttcaagatatatctcgaacaatttacgtattattttcacaaattttgttccacaataatttaggttattttggtttttgttgttgttttttgagtctgaaatttcaaatttgtatttttttttaatatatatttaggcaggaatctttaaaatgaaaccaataatatattcactaagtttggaaaaaaaccaaaatttcataattaacgggacaatgactaagctgtggaaaagataacataatctcccaatgactcaatgccacgtataagatgagaaccgtactcttctgtgttcgtaccagatccctcaaaaggcgggaagaaaccatcaagaaaggcacattgctgacgtggaatggaatattcgaacacgcatatccagctccttcctcctttttcctcatatcccttttttctcaatataaatactctgcaaaaaaacacccacaaatcctcacacaaaaacgttttcgaaatccctaatctactctctcttctctatatctctactcttgagaaatggctcttagaactgtgctcgtctccgacgtccctagcctcccggattctgtctacggtttatccgaaggtatttctctctctgtctctctctctctgtttcgtcggctattgagctgttttcgtttctgatgatcttgtctttgattacaggtttagagttgagcaaaccgacgtcgtttaggttaccggggttttcggtgattggacatagaggaatcggtatgaatgtgttgcaatcgtctgatcggagagcgagaggtgttaaagaaaactcgattctttcattcaattccgccgctaagtatccgattgatttcattgagttcgatgttcaggttggtactccgatctatgtaatcaactttaggatttgaatgagacagaggattggggttttatgtttatgtttttgtgtgtggaagaagattctgatttagatatctatttatagattcttagaaattgaaatgaaaaataagaacatgaagatttgaagaaagagagagagatggcacatagagaagcattccaagctttgtgtggtttttcttttgtaggtgacaaaagatgattgccctgtcattttccatgatgatttcatctactctgaagaaaatgtaagtctacattgctttcttcatagatcttggttttatatttctggatcttgtcttcatggatctggtgtttgtgtgttatggttcagggtattgttaatgagagtagagtgacggatctgagtctgtctgagtttctcctttatggacctcagaaggaaactgagaaaatagggaagacactgatgaggaaatctaaagaagggaaggttttgaaatgggacgttgatttggatgattctctttgtacgttgcaagaagcttttgaacaagttgaacaaactcttgggttcaatatcgagttgaaatttgatgatcagaccgtctatgaacgagagtttcttgtccatatcctgagatcagtactacaggtatgctcaatactagttccgtattcttccttctttttcttgttggagtatgaatatttattagggtccaatagaaccatttgttaaaagtgctcacaattaatttgatctcttgagacctttttgtatcaatgctgttgacctaaaactgctggtccaagttggatctagattgattgagacttgttaacttattattttctgttttttccattctgtgacaaaactacaggtggtctctaattatgctaaagacagaccagtaatcttctcaagtttccaaccagatgcagctaagcttgttagggaactgcagagcacttaccctgtatgttccattctcgctcttttctttgataaataccaaaaaaaacttcttgacttcttcatggagaaatcacttaagatttaacaatgtcatatgaactgaacccaggttttctttctgactgatgcgggaaacgagattcacaatgacgagagaagaaactcgctcgaagaagccattcaagtttgcttggaaggaggtcttcaaggtattgtttcagaggtaaaaggagtgttcaggaacccagcagccattagcaagatcaaagaatctaacctctctctcctcacatacggcaaactcaagtaaaccttcttcacttttcttaataaagtctaaagaccttaagtttgtttccttattgctgttggatccgatcactgattaatcagattttaatcttatttcaaatattgtgttgcagcaatgtcggagaggcagtgtacatgcaatatgtgatggggatcgatggagtgattgtagattttgttgaggagattatagagtccacgacacgcatgatgataagaccaccaccatcatcatcaccattaccatcaccttccaaggatgatgatgttgccattacaagacctgagttttcacagaaggagatttcttttcttctcaagcttctctctcagttgatacaacattgatatctctgttttttcgttttcttttatcccaagtttctttttagggtttgattttcttattgatttaattattattctgggtgtattacccaatagttggaattgtaaataattataaatttatttctttttacaaggattaactttctcaaaagaaaatcagctattttttaattaatcgaaaaatttagaaaatatatacgtgattatcttttatataagcttttaagatatgtcacccaaaacaagctaagcccaagatggatctagactttgaaactctcattttagggcaaaaaaatggttttaaggttgtgttatggattatcttttatataagcttagttatatcactaggtgaccaattgtaatgtgctttcggtatagagtcagagtgatatgacgggatacgtagtttgtaagcgagaaggtgtttttcttagtaaagcttttcttatccgcaagaagattgacttcaaacatacagcttatctcttgtgagaggtcaagctttataatggagtgagatatgtattttcagaagtcttaagtatggacataagatattagtttcacatatttcaatttttgaaagtttgcaagtaaaaaccacaaaacaaacgagtctgtaggcacgaccgcacgagtgctaaagtccgctgcatattgatagttcgaagacggtgttttgtcgatattattcactatttacgtgtcgcggattgattagcgactaaagaattagccttaacatatgttttgttgaaatatattttgagtcaagattattcttaattcccaatagaattttgaagtatttgtctttgttccaacctttaaaaggatgcttgaagttccaatcaaactacaactcatgctctctccctcttctgccttaactatgaagaaaacaaaagacattcgtttttatttttctcttcagtatcaaattcaagaatccaattccaatagccacaacataaattctctttgatataataatctaaaattagaaactgattcacgtctctaaattaaaatttctgatctattcaagtaactgttcttatatgagttttgtttccatacatgctttgatttaaacgttttctactacaattatatctacctctcaacactggaatccaattttctttatacggtttatatcaagtattgattgtttttgtccaaaacaaaaaccaagtattaattgatatagctatagtacactcaaattttaatttgctcactataattctgatattactttgaatagaaattcaaaattatctaatcacaaagttcagtttatttataatatggaagctttgagcaaaataatgtaaatataattataaaaaataattagaaataatttaggacaactgaaaaaaatatggatttcggtctataagtttctattatatatcaaaggaagtacttttttttttttgataaaccctaaatatcaaaagtacttttcgtttttgccaaatatgtttaattacttattttcgagatgttatcttccatttttttcagtatcattgtaatcaatgataattataacaaaatcacgatatgaaaaaaaatattccattaattgatgtaaaaaaaaaagtcaaatatcataacattaaaatgtaactttccccttatttactaactaaagctcatgcatgcgtggcgtgatattatctattcaattccttttctaaaaaggttttcctagtcctattaggattaggaaacagaggttgctatataaatacttgattcgctcgcgtttatctcagtcacaaaatccctacttcttcgtaacaacttactaatatataggcagcttggagcttattcattggacgacgaactctttttcttcatctctttctgttttctttttctcttcttttgttctgagaataatgggcccaacatacagagctttgccagtaggtatgaggtttcgtccttcagatttggagttggctgtttactttttgatcaagaaagctttaggacttcccatgaaagctcttactgtgcccgacgaatgtaatgatatcttctcaacacatcctcgcgacttgcctggtgagaatactttctccgatccttttgacaaaacagatttgatagagtttttgagtttcttgatttaactttggctacttctttcttgcgtgacaagctttattttgtgaacttgtttttgctctgttctttcaggctatggaagcgaagagcattggtacttctactgcaagaaacccaagaatcaagttactagaacgaaatcttataacctctggataccaacgggagaaaaaactgatgtgttagatccaaagaagaatggcggcgaattagtcggtatcaagcattcgtttactttcattgagaacgaagaagaagaagaatctgataacaagaacggcgatgaagaagaacctccacaatgcaattggttcttggatgaaatcagcctcccattgacagttgtggatactgattggactctgtgtcatatcttttacgagaaggttaaacctgaatttggtaatttgcatatagtcgaatctgaatccgaatccgaagaagaagaagaagatgaaagtgttgataagcctgcagagagtttggatagtgttaaagagaaagatggaactgttcttcctccacctcctgctacaccttagcttcttcatgatatagtgttttactgttagtgttggtggattgtagattctctcaaaacctgtcaacgtccaaatctttattacctctagttgcatacctattgttttaaagatgcttatttcacttcaatatatcggttaggttttttttctttttctttccacaatatatcgattaggttatgtggctcgagagactcacttgctcgctgatacaaaagttccagaacaaagagatcatccctcatctaatggctcatgttagcaattgtccgcctaccttgctaaattgcgctcctacacctaaagaaaactattgatatgagtcgtagtaataatcaatcgatctctctattccaataattgttaggagggtcataatatatagaagaagatagatgatatatgttatataatatgatatattgatatgtccaaaaatcaaattgttcaaaagccttaatgaaaaagagaaagagatgtttgaccagcttaaaaacttccggtcaaacacctaaaaaccttttttccgcctttgtcataaatctttgactttttgactttttccctctcaacatctcttttcgagatttcaccacgtgtcaaacctctcatctgaaccgttcatcttacacgcgtgagattatctcattttctcctcctccgtctttctgctgtttcttcgatctgtttctttgaccagaaaaacaaaagcttcaaactttttagcttcttcttcttccttcaatgtcgttgaacacatcccacgatacttgacaaaatatctcactctttcgtctctctgttttcgactcgcaaaccatcgaagcgccgattaatcttagaggaatttcttcgtgattagggttttgtttagttctatctggagatatgatgaaagcttcgtttggaaggttaagaagattcgcattacccaaagccgatgcgattgatataggagagctttttcccactgcacaaattgaaggtcttgctcgagccgccaaggtaaattttccctcttttcactgatcttctctcatgattctctgagatttagttttttttttcttttcaaaatcgtgagttattgatcacaagattgtaacttggatgagtttttgtggataaaaatttccgatcaaaaatcacaccctaattggaaatttcaggacttttacagttatagaagtttgttcattaatttctacttttatagacttatcttatggagaaatttggaattggtatttatatcagttatggtaagaaattgatattgtaactgatgctagttgttgattggttgaactagaactggaagatacattttatgacctaagcttattactcaattgttttcaggatatgcaagatatgagagagggttatgatagattactcgaagtagctgctgctatggctaatagcgcatatggtaaataagcattcaacctttatgaatctccacttctatagcttcatgtatgaatttggaaggttgatgttgtcaaacattgtgactgtaaagtgtacggttgcttcatataagatggggcttgctatacccgagctttattggattgaattagctttcattcatattgtttctagcggaggtcttcgataatattatcattttgcgaaatcttcatttttagtgtctctagaatttcccattaagtactttcttaaccttgtgagaaacctttgttgattcatataagaacatcagtatattgtcagacctcatcattttttttttttcttttgttcggtataatcagagttctctgaatcattgggggaaatgggctcatgcttggagcaaatcgcgcctcataatgatcaagaaagtggtaagattttgggtatttagctatcatggacgtttgtttgtcctgtttccatgctgtaacattgtttcttctgcaggtggaattcttctaatgttaggtaaagttcagtttgagcttaagaaactcgtcgacacttatgtaagttgtatgttgcaactttgtacttttcatgaagcaatatgtataattatgaacaaaactgaggtagttcttgtttcattcttttttttctccttcagcgttctcaaatattcaagaccattacacgaccttcagagtcacttctcagtgaccttagaactgttgaggtatgccctagctttggtcttttacagtcagagattatttaaaagccggaaaagttcacggtttgagcaagatttaaagctaaagtgaacaaaaaagcacaaccttaataaagaccgctgattttgtcgtatttccttttcttttcctctcaggatatgaagcaacaatgtgaagaaaagaggtcagtgactttctgcaatgtagttttttattctctcggttttgatttgataaaatcttgccttaacctctgactcttacttacagagacgttgttaagcacatgctaatggagcatgtgaaagacaaggtacaagttaaaggcactaaaggggagagacttattcgccgtcagctagagactgctcgcgatgagctacaagatgaggcgactctgtgcattttccgattgaaatctctcaaggaagggcaagctcgaagtctcctcacacaagcagcccgccaccacactgctcaggtaccttctgaataaaattactattctatgtaagaaaccttcctgcaatttctcaatcaatgtcgcttatactttcttatttttgcatagatgcatatgttctttgctggtctgaaatcgcttgaggcagtagagcaacacgtcagaattgctgcagatagacaacacatcgactgtgtgctctctgatcccgggaacgaaatggattgtagtgaggataatgatgatgatgaccgacttgttaatagagatggagaactcagttttgactacataacaagtgagcagagagtagaagttatatctacacctcatggatcgatgaaggtaatttggattttaaattgaacctgaattgggaaagaacaaatttattcgttaaaaagtaagctcatgtactgccatttctgtatagatggatgacacagatctctcgtttcaacgcccttcacctgcaggatcagcaacagtatgttcttttcttgatttttatgtcagatttaacatttcatgtcttgagaactctcagttagtcaactgtatgagaactgcattggcgattctgtgttgaagtctattctctgatgcaggtaaatgcagaccctagagaagagcactcagtttcaaaccgtgatcgcagaacgagcagccattcagcacctctgtttccggataagaaagctgatttagcagatagatcgatgaggcagatgactccatctgcaaatgcttacatattaccaactcccgtcgactcaaagtcctcaccaatcttcacaaaacctgtcacccagacaaaccacagtgcaaacttatggcattcatctccactagaaccaataaaaaccgcccataaagatgcggaaagcaacctctattcccgtcttccccgtccttcagaacacgcattttctggaccactcaagccatcctcaacccgtcttccagtaccagttgcagttcaggctcagtcatcttctcccagaatatctcctaccgcttcaccgcctcttgcttcttccccacgaatcaacgaactccatgagcttccaagaccaccaggtcaatttgcaccgccacgacgctcgaaatctcctggtttggttggtcattcggctccattaacggcttggaatcaagaaagaagcaatgtagtagtgtcaaccaacattgtagcatcaccacttccggttccaccgcttgttgtaccgagaagctactcgataccttctagaaaccagagagcaatggcccaacaacccttgcccgagaggaaccaaaacagagtagcctctccaccgcctcttccgctgactccagcgtctctgatgaatctcagatcgctgtctcggtctcatgtcggggaagttgctcagagcggactaattagaggtaacggaaacaaaactaacttactgtatttgtaacatatctttaatcatcaatgattttaagacatggtttgtttatattaacgcaggagtaaaactgactgaacactgaccttgtaacggttgcgtttgatcgaaacgttagttgtagaaactagaaagagacccataagtaaatatagaagtcgatatttttgtatttcgttagttgttgaatctcctaatttgttcttaagcctccgatattttggatcttttttatccttgctttgaaagcaacgtcaacggaaaaattactttcttacttaactctgagaaatacgattaaaaatgagaatttaaaggaaatgatacacttttaaattcaagaaatcgataaaccttactataattataactttttgatcaagaaaatagatggatattttctttctaattatggattttgctttacacaaagggaagtgaaaatgacagtaacatgaccagaatttgggtagagagataggaagacaaatacgtttaccttgtcacgtgagcaacccaaactggaaatcacgtgatgattctcactagcaaatgggccaaggttagtaggcccatgtagaggataagtacctaacttctatcttctctcctctaatcttcttctttagtttgattgatttgatttgaatttttgtattcaagagcattattctctttgattgtgtataatttgcatcaaatatcctaatttaaagttttgagatcgtgatttgttgtgaatggcgtgaaactgttgacgaattgacaaatttttttttttaattttttgatatttgtaacaaatcttttaatttattttgccgttggggaaaaaaacgataattatgccaaatgtttctaacacactacagtttccagaaaattaaaggttaaaaaaaaagttaaaaaaaaaggagagctatagaaatggtgatgcgatgttgggatttgtttataaagccaagagacgaagaagaagagttctcgatcatatacattctattcctttttcattttttctctttcgaatcatttcttcttcttcgtatatattcttctccaattcgttcgaattcagtggtgtcgatttttgggggtttctgtgaataaactttggattgcgtatgaaacgtttcatccacagattatgtaattgattgctccatcggtgcgtttgatgatcgattattctcgttttctttgattttggtgttgctctgatgatgatgatgatgatgagattatctcttgatttgattttatatgatcacacaaaatgttctctcctgtgcgatctctcgttcagctcgaaaccttttggttgatcttcaattttaggcggaatttgtggttttgtgtgcttagagatgtttgcttgaatcttatacactgagagcctgtgagaatcattttggtttatatatataaacatctctttacttcttttgaatcacatcatctgtttgttaatctgtgcattattgaagcttatgtactatgttcttcattgattctttagcttaagattggtcctagtttatatgatcatgatctgtttcatcatttttgttacttctacaaattttgttgtggacttgtagtagtgatgcagctcaataatctgctagctaagttttttggaatgtgttggatatgttgttgattcttcttctccatttttgttcatttcaggagactatcagctctgttttctctatcctcaacatatatgttgctccagaaatcggtcacataaattttatgtctcatctctttaatgtggacatcaatggatgaactgtggatgcctgtaacttatgcttagttctttattatctagatcaaattgattcagtggtaatccgtcttcttgaaaggatttggtaggtaattggttggagtagtgtatctcagattagtatcgatagagaaggtctttaagagctcacaggcagctatataattgagtaagtgataaggtcctttgagcattggaatgggtctgccacacactgcttcaaatgagcaagcccatgaagctttgtctgctccaagttgcagcctttctcaaagtctgccagtaaatgccacttcctgtgattcagacggtattaacagagggaacgaaggaagtttccgcttctctattggagagtctgataggaaaacagcagtggaatcgttggagtttagagatgacttatggagattccatgacactcggtctgttacctcattacctgctcacgaaccgacttatgcaatggataaggtaaatttgagtcgaactgaagttagaaggatagttggtttcgaatcaaatgaagttagctcagtgagtaatgaatacacaagtattactgttaatcgcactggtggatctaatgaggtagatattatcggtggatcgttagtcaggaaacgggtaagctctcctctgaataagcttttccctgagaaatttagaggcgactcgcttgatatcagccatagcaaccaaccgatgacttctgctggtcttcctaaaggatttcacattcctgttggacaagatcataaaaaagccaatatctctgggagattgcgtctcttcaccacttctaactgctctgaatgggggaatgacacatcacacacgggtaaactttcatcgactgttttcacggatggtccattgcttgactctaacgatctgcagcctagtcaagatgtacattgcttatattcaccagtacatgaaacatttcaagtacctaataagccattaccatgtcatagaaatatatctgtctctccgccactttctctgtcacctcttgggccaagattttctgagaggatgaaagctcttcaagggggcctaaatgggaatattttcgaagatgatgtttgtttaaagaatactggtgaagaagcagaactcaggactggtcacagatcgtttgatgatactaatggtatccagagagccttttcaatggatagagctattgaatcagttcctacatctccatgtaaaaggtttagtagaagcttgagtggacgtcccattcaaaggtcattggttggttcttttgaagagtccctcttttcaggaagattatcttatgggcaagctaatcaggtctcactctaccaaaacccatgaaactcacaaaacctcgaagcaaaaactctttgttgttaaaaacattaaacatttaagatttgactcacttactctcgttttgttttgttctttgcagaagatcgacggttttcttgccattcttagtatagctggtggaaacatctctccaaaatcacagaagcttccattctcagtaacaagtgttggtgatgattgcttcttactatactatgcctccatagatctctctggaggatcattgccaagcaaattatggggtcagaaattgaaaaccaaccaaaacaagtctgatgctcagaccattaataaacgtcttcggattcctatgaagggtcggatccaactggtacccacttgtctctttgcctatttttcttaagaaaatttctcttcataatgtaataagatcactggataacaaatttatgataggagcttctctgttttgcaggttctgagtaaccctgaaaagacacctctacacactttcctttgtaactatgacttaactgatatgccacacggtacaaaggtaacatcaaaaaccttcctccccttgtttcattcatttgctatcatcccgttttagatttcgattgttgcttgcgtttacatacatttagtgtatattttttcaatgaaccattagccttaagcaaactcgattttcaaaatttacacaaaatcggttatgtactaattcaatatttgatcgaattcttgtgtcatggtcagaccttcctgcgccaaaaggttactctggcctcctctgttccgaccaaagccaaaaaatctgcaaataaaggtagtgaaggatccgagttagtggatgaattgcattcgccgaatgagtgtgggaacaagaattgtcgtgagacgtacagagaaacaggacaacgctgttcaaagtctggagtcttacgctacgcacttcatcttaaatttatttgccctttacgcaagaaagcctccaaattaggtcagaagaaaagcttggatgcaggagatgatggagagcggagattctatttatataatgaattgagagttgtgtttcctcaacgccacacagattctgatgaaggaaaggtatgttagaacaatttgagaaatttattcaaattcatttggtatagagctatttgaacgcaaacgcaaaacgctgatgttgcttttgaaattgcagttgaatgtggagtatcattacccagaaaatccaagatacttcgacgtgtaaccaattgaatttctgcttcctaaaccgtttttctgttttagtttttgttagaagaaaataaaagaatgttgtacataaaatagagaataatcatatctatattgcccattagcagctataatgctccttcaagcagccatactgtctatgcttcttacctattaaagcaaaccacaatgggtttcgcactttctgctggaaggagaaagcttaagttatatataccaaccattttttacaataacacacccaaatctacgtgtttaatcaccacagttaaataagatatcttgtagatagagctacaacacagtaactaaatttaatgatttcggagatcgaaggttgggatttgggaagagaaaccatgagtgaggtaatagagggacctcggcaaaatgtttctgtaaggatcagtttctcttttgattttcaaatagttcatacatctttctacctcagacttgacctgcaagtaaacctcatgagctttctctttattgtttagctctttctctctaccttctgtgtctattggtttcccaaagtatgcgtaaaaccgtcccgggatctttggaactattccaggtagatgtaaatcttgttttcccaattcgccttcttcatcgttcctaaccatatattatcatcactatattatcaaatgttttatcaggttcatgtgactttatctaacagagtcgataaaaatcatacctcaagttaacagagtcttgtgttatctcttctataagattcttcaagaaagggatcttcatttgatcatcataatctaaaaccatctgaatacattccattattgtacaaaggagttactccaaatccgattgaagcaaaaggagttttaagacgattttaacttttaagaatgcatttgatcattgtaatccatgaccatctatgaatcttccattgctcaaacgagttaactcaaaacccaattgaaacaaaatgagttttaagaatgcaggtgttgaaagtagaacggaaaagagatttttttgcttacttcacaaagatcatcttctccaacaactccaaaaggaatgatttttgctccaaatttagatgctatccttacaaactccgaatgttctggccaaaataacttgtattcttcaccctgttataaaatgtgtctttagatataaagcctcaaaaattaaaatgaaggaaaatatggaaaatacataatcacctttctgtgcaaagcttcacgaacacccccagggtacaaagccacgtgagcctttgaacgaagtagtttgtagaaattcattcccgagacgggaactgcgcctataatcctaaataagtcgtacagctgcatgtcagggagttttgagccagtttttttggtaaacatcaatggatgtgccagtcctcgcaatagaatgttcctttctttcaaaaaatgaagtgctattgcatgcaactccataccaagcaacatgtgattgccaacatacagaaccggtccctctgaaggtattcctgcaagcgatcttaccactgcaccattctttagagttgaaagaaagactggggaggtaacagcagttagcaatctggaccagagaaattaaaaaagaatactacttcagtatgtgcttatggtagcagaagcaaatattctattctagctaagttactatacaaaatattttcaagaaatcatttgacagagaaattagaaaagtgataacatctatgacgcacagatattaatgtgataagtgagtcattttacctttgtgattcttcatactctttaaactcaaatggggtaggcagaatgtaatccgaaatgtaatcaagtgacttcccacggcgataataatacgcacgcttgatgatactcaccagatctactccatcctcctgacatgacatcaaatagatcaatcgaactatatgctaattaaccaaacataatctgaaagtttttctgtgccaaatctgatgcaagaagtttctattggctataaactcgtagagactagatttgcttgtgaatttattaaataggagtcatttacacagggaaaacttcttgcatcaggagaaagggctccttaccaagaagaggaactgtccattattctcaagctcacgaacttcacatcttggcaatgcaccacggagtctttcaatgtcttccttgttcattaaccattgatcacgtccactgcaataacacaggcttattaaatctacacaactcagcttccaattaactaaactcattaaaaaaaaatagatcactgaacttccacttatattcagagcagagaagtttactagtgtattaacctcagaagtatcagtgtttgggcgttgactgtgtccatctgagaattagcagacgctgaagcagacttaagcaattgaagcttccatagaagtgtgtccttgggaaagattctaatcagagtctgtaaataatttgtttccgtgagtttatctcatgactagagaaagtaaaatcgaaacaacaaaatgaagaacaaccaagagcacattactagttaaataatatgaactgcaagacaaacaacatgctaatcatttgttctcctaagatagagtcttacaggcagattagatgaagttgcaaagaagtctcctaatagccctctacccatctgcgcggcatcattttcattgagcatagtctcgaacatttctgtcaatggggaagctatgtaataaaacagttagccctgactttgaagcaagaccttacactgaaacccatctttgagacagaattttataaggcacagtctttgcgtctaatttttttttgctttacaccgatatacaaagggttgaaacttcaccttggtaaaacccaaaattctctgttatcaagccgggaactccgtcaggcaaaatttccagtagggccaatacaggttgcaacattaagttggtaaaacgtgtgactgcatcaaacagagtgtattatgagcacaagatgtatacactccatgagatctacccacacaaatcccccaaaaaaagaataactcttttagcatgattagaattatgctttaccagcaaaggttatataataaagtgtgcgctttgctcataacacttttctgagcttgcaacaatgtggtttcataaatggcctaataaatcttaggaaacaaaaaaaaaacacacacacaataacggtttcaattctcaatgatccagaaaaaacgagtagtcttgttgtacctggattagccagaatcaagacaagatcaatgtcagggttactggctgcaacatccagagcaagagaagctccaatagattctccaactatataaatgggtctatttgggaaacggtagtgctctgacctaactgtcttctcaatgagcttcccaatatctgaaaacaacagtaagattttcaccaacataagattacaaaacgctaatgcgacttcagataatgcttaagaagcacctcgagcaggagtacgatcttttactggaaagtgaaggcaccatatgtcaaatatcctatgatcaataaagataatgagatcatcaacaaagagtacatcactagatctctacttctctttctaaagtaacaggagctgtgcacaatgaagagcctatctcaagctcgaaggcaataaataagattgaatcaaacaagacttactctccaagcctcttatgctggcgaatgagccctaatccagttccatcgatccctgaaattttgaaaaactagttttcaacaacaattccccttcagcccaatcagaataacgaaatcaattaaaggaaatatctatttccagccctaaattgaaagaaacagaccaggtaagtagagaagaagaggagattcaggagcacgtgcgccacattccaacggtgagaaccaccgtggcggaccaccatctccgccgtctgaattaacgaaatctccagcttccgttaaaaaatccgtcaaactcttccgttcctcaggccgtacttcctctgtatacgaatacggattcaccgtcaccttcaacttcccgccgattccgaatttcggattcgtaagtcgttgggttgaattcgagtagagaaaatggagattataagcctttgtcgaagctgaggagaagaaggtgggagaggttccatcagtgaaacgaacagtagtagtagtaataatcgccatagacgatcagagaaagttggaagaaggtttttgttgtttagcttcttcatctctgataggtcgaacacgacaagctcgtgcacacgagaaaaacgattataatatgccacgttgattttttcagtttgaatattttcaaatttgataaatttgcttaaattaaacacgacaagctggttcggtttggttaaaccaaatctgggttagtttaactatttattaataaaactaacctcgaatattacacagtttattgtacattgcaatattcttcacccaacactcccaaaagttactcaattctattaaaatagaaaatagtcaaaatacacaaaccttcaaggcttcaacgtaaacgttctagatatgttgtaagaacacaaacttgcatgaatttttaacataacaccccgaacttactcaatttctacatcaacagccttgtacttttagaagttacaataacatatccaaattccacaaagttaattaagtcacaaatccagtaactaaaattattgatttcggaggtcaaaggttgggacttgggaagagaaaccatgtgagaagtgatagagggacctcgctaaaatgtttctataaggatcaccttctcttttgatcttcaaataggtcatacatctttctacctcagacttgacctgcaagtaaacctcatgagctttctctttatcatttagctcgtcctctctaccttctgtgtaaattggtttcccaaagtatacgtaaaaccgtcccggcatcttgggaactattccaggcatatgcaaatcttggtttcctacttctccttcttcaccgttcctaaacatatattatcatcattccaatgttatataacattcatataattacatttaacagagagtcgaaaacaaaacaaagaaacaaaaactaaatacctcaagtaagtagagtcttgtgataactcttttataagattcttcaggaaagggatcttcatttgatcattgtaatcgaagaccacctgagaatcaattccatttttcaaaagagttttaaagaattccagatactgaaacgaaaacgaagaagaagagattttgcttacttcacagagatcatcttcaccaacaaccccaaaaggaatgatttttgttccaaatttagatgcggtccttacaaactctgaatgttctggccaaaacaacttgtatacttcaccctgtagaaatgtgtcattctatgtaacgcttcaaagttaaaacgaaggccaaatatgagatatctaatcattacctttctgtgcaaagcttcacggacacctccaggatacaaaaccacatgagcctttgagcgaagtagtttatagaaattgatatttgagacaggaactgcgcctatcatcctaactgagtcgaacatatgcatatcagggagttttgagccatattttttggcaaacatcactggatgtgcaagtcctcgcaacagaatgttcttttctttcaggaaatgaattgctgctggacgcaattcggtaccaagcaacatgtgattgccaacatagagaaccggtccctctgaaggtattcccgcaagtgatcttactactgtaccgttatctagagttgagagaaagactggggaggttatagcagttagcaatctggaccagaaaaataataacagcacttcagtatttgcttacagtagcagaaggaaatatcctattctagctaagtagtactatacaaatattttcactacatctaagtcaagaaagcgtattacacagaggatattcattttacctttgtgattcttcatactctttgagctcaaatggggtaggcagaatgaaatcagaaacgtaatcaagcaattttccacggcgataataataagtacacttgatgatagtcaccagatctaccccatcctcctgacatgacataaaattatcaatctgaaagtttgctgagctaactactagttataaacagatacaagaaaaactttcttacatcaggaaaaagagcaccttaccaaaaagagaagctgtccgtaattctcgaacttacggacttcacaattcggcaatgtacaatggagtttctcaatgtcttccttgtttagtaaccactgatcgcgtccactgcaatatacagtcttattaaagctacaactcagctgccagctaagaaattttttgacgaaaaaagagatcgctgaacttctacttattgaagcagggaagtttacaagtttattaacctcaaaagtataagtgtttgggctttgactgtgtacatgtgagacttagcagatgctgaagcagacttaagcagttgaagcttccatagaagtgtgtcctttggaaagatcctagccagagtctgtaagaaatttgttttcgtgagttaatcacatgaaaacatagctgaaacaacaaaatgaagaacaaacaagagcacattactagtttaactctataatcagcaagacaaacatgcatctatcatttgcttttctaaaacaaagtcttaccggcaggttaactgaagttgcaaagaggtctcctaatagccctccaccgatctgtgcggcatcagtttcattcagcatagtctcgaacattgctgcaaacggataacctatataataggaacacagttagctgtgatttgaaccaaatcctgacactggaaacccatcttggagatcgaataaataaagaagtctctgcatctcatttgtcttcgactggaagatatacaaggagttgaaagttcaccttgttcaaaccgaaaattctcctccaggaagctgggaactccgtctggcaaaatttccaatagactcgataaaggttgcaacatgaagttgttgacacgtgtgactgcaacaaacaaaacgacacgcttaaatcataaagtacgtaattttctcatgacacatatttgagcttgaaccatcgttcttacataactttggcctagaatatctagagaaacacaataaaggatctcaatgatccagaaaaaacgagtcttgctttacctggattagctagaatcaagacaagatcaatgtcagggttactggctgcaacatctagagcaagacaagctccaatagattctccaactatgtatatgggtctctttggtaaacggaagtactctgacctaactgttctctcaatgagcttcacaagatctgaaaacaacagcaagagtttcatccacataacattaatccatttggtttgggatccgtactgaaacaacagagaatgctgacaaagaagcacctcgagcaggggtacgatcagtgactggaaagtgaaggcaccatatgtcaaatatcctatgatcaaaaagataatgagatcgtaaacaaagagtattcagtagattccttcttcttttgaagtcattttgtactacttattgataaatcgaagttttttaaacattgaaacaggacctgtgcacaatggagagcttatcttaggctctaaggtaataaagaagattgaatcaaacaagacttactctccaagcctcttatgctggcgaatgagccctaatccagtaccatcgatccctacaatgttgaattcttagttttgttaacttccattgttccaatcagaataacgaatcaatcaaacgagaattgatttcagccctaaattgaaaggacgaaccaggtaagtagagaagaagaggagattctggagcgcgtgcgccacattccaagggagagaaccaccgaggtggaccaccatctccgccgtcagatctaacgaaatctctagcttccaccaaaaaatccgtcaaactcttccgctcttcgggctgcgccgcctccgtgtacgtatatggattcaccgttgccgtcaccttcggtccgttccgtagacgtccatcgattccgaatttcgggttcgtaaaccgatcatgcgcgacggaggtcagtcgtcggactgaattccggtagaaaaaagcagtggggtagattccatcggcgatacagagtgtgggaaccgccatcgatcagaaaaatccgagatcgagagaagaacgatttcgtagttgaacttcttcatcttcaagaggtggaacgaacacaagaagctcgtgtacacgagaaaacagcagtgactactccgccattcttttatcgagggtccgcatttctgtttgaattttaaaatcaatttagatgctgctgttggaatttggcacggtttaaatttagggtaacttatgactgttaatattataatttatattattttatattttagaaatataaatatcaaaatatatattttttttttgacattagcttttgtattagatatttgttgtaacatggtcacgatgtaaattattaacaattagaaagggaacataagaatcgatgaaaaaaaagaagaagagagggaacgtaagaatgaaagtaaaagctataattattaggtaatccatatctggctagtgtatctacagtcttgttggagaatgttggatctatgctaaaacaaaaactgacattttttcttggcattggttttcgggtgtaaaaacctatattgcaaaaaatatgagattcaaaagcttaaagtccgttgcaaactaatcgacagtcatcttttcgttttgcgattcttcacatataggaatccaaaagttgtttgcagctatatcctctcaccaattctccaagattttggaagtatactaaaaagggacactggttttgggtgatgatatgttgtggtctgttataaaagggcataatttcaaaacagatatagttggtaaatttgtaaagaatcctttttggatatacaagaagaggcagctcacagttcacaccacctctctaagagtttcaatgttggtaagaagcatgtttgtcatctcagtgatgttaatcccatatatttttggacacttgtaacctttagtttatttgataaactatcaaattttggtatatttgatgaatcgttgcaatatttgccctttataaaatatttttgaggatatcaattaaagttaaatttgttcaattagagttgtatattgtcaccatgttttactcgtgtttataatcacaaactcttcgttccaatacttatatcttttacaaatggtgtctatttaaactttcatgtatcttaactttatatatatattttttattttcttagaaaaattgtgtatatttattattactttttgtttgatacattttcatataaatttgtacttttaactatatagcattcatttatacattttgttatctatatcatcatttgtttcctaaataactgtcaatttttgtttttttgtttttttcgaggattgtatgtcgttaacataacacagtcttaaatgtttgtgtctgtttatcataatgttttaatgagtatatttatgtatttctatcataattgtttaagaaattaaaaattgcgtctactcattttatttttttctaataatttttacttatggttgcattttttcaccatcatggtttctaatgtaatattataaatcttaataaatgcttgagagtcagagtaagctatttgtaacattaagcattttacctttaattttttaaaagatataaggcccaatagtagtaagagctcataagaccgtttgtatttctgaggtttatttcttctctcaaaaagaaccgcggcgattcgattgaaccaaccggttagaaaaaccgaaccggtgaagtggctcaaaaaatggattaaaggtgcttcacgtgggcagattgggttgtcgctcatgtgagcactacttgatatgctcgtaatcattacacatccttttacttattatttattttctcactacatcattaatctatagcactaaaacatttgacaaaaagaattatacattagattcttcatcagagccacactaatagtacagtttgtttaatctttgtgcaaaagaaaaagtagtacaacagtttgtttaatcttcgcatatgatttcatttgccgatttattatatgaactaaatacaaagttatgttctttttttttttttgagcaacaatacaaagttatgttctttatgaaaacatagtaataaataaataaaatacttttgtgtggttttaaccaaaacatcttagattataaaaatatattttgatgattattaaagtgatgaattcaatattactatagtaataaaagtcactagtgttttgagaaatttccaaaaaataaaaaagtcatcaacgttgtaattataaaaataaataaaatcatttccaactaatcctttttaggtatataagtatattcaaacaattaatttgatcggtgactttgtaaaactgtgaaatattgttttataatgtgcttagtgaattatttaccagtcacattctgcatgtctcaagagattagatgagtgacattttatgacgtaacacaataatgttatttatacgtttacaagaatgaaaaaaaaaatatctattcatcatttgtaagcacacatgagaggattaaagctttacagaggaaaggaaatgtcacttttactgtcacatgctgaaagtgagaaatggctgagagagaattatttgttgaattatacttatcttatttctcaagccatcaaccaataataagaattaagaaataaagactagtatcaaatcttatattggtggtgtaatgctgatgacctcttagccactccgtttggcttcttctggcatagagatatcgtgaaatatatatgtagactaatgagtccaaaaattggattggtgcattaattatcatgttaaaaatttaagatgcaacctatctagagtgaaacatatatatactttgatatagaactatggttggtgtggactcgatactgaaacatataaatatgattaattcgattgttaggtttggatggtgcaatcaatatgctaaatgattctagtataagtaatctaattcttactaactttgtagctgcaaaatgtatattctagtataaatatatttcttattatacattctcaaacatgcacatcgtaaaatgttaattttgatatattaagatgtttaaaactaaaggcaccaaaacccaccaaaaaaatttaagagtctataatagattgtgtgtttcacaaatttggtatactgtgtttgattgcttggattaatgcttatagaatattaagacaaaattaacatttatagcaatagttggtgtgtcatgtcaagtaaacttatgttgttagctgtcagattgaagcagatagatagagaaaaatcttttgcgaagcagttaggtatctgacgttggggcactccgtgacttcatttatgatttgtttttatgaactctggtctatggcatcgaattatagtcgatttttagagggaatataatgtacaaacctacaagaaaaattaaacaaaaacacaactctaattattattcaattgtagaatttacaagaagacatttttattaggttttgtagtttttttttaatgaaaacacttaaacctgccctaaaaaccaaacaaacaaacaaatgaaacaaaaaaaaaaatggatataactactacgtacgagcttttttttttttaatacaattacatacgagctatatggactataacatattaattgtttttgtgtagctatgtaaatctttatttagcatatagaagtgcacctaaattaggtaacatatcctcctttttagtttcatacattaaaaaatagtaatttcttaaagatcaaataataaataagaagtccgaaataaagaaaaaatggaccttaaattctatttttcaaatgaaatactttcataaaattaaattcaactaataccacaaagacaccaagtttttggtagtatcccaaaaggcctatcaatgaattattttttttgttattaatttttagttaattttgttgtataagctaccaagaaaaatacagctgcttatataccccacaagtattggaatttagaacgaaaagacaaaataataactataaggttttgatgtcacacatcttatgaattggcatgttccatgtccgatctaaccaatgatggctttaattgcttttgtaaaataaaaaggactggcaaaactatgccgcataattttcgcaatgatcaaatttctaatcgtggttggttaaacataatattctctggtaaatttataaatagttatgatgatatccaaaagtggttaaaaagtaaaaataataattacatagacattaacgttaaagttgactatttccttataagaaaaaaaaatgaccgttgcctattttattgtaccatcatcgagaccaacgaccatgcatacatttatgtaactgactatactacctctactagcccgctttacgaaagtgacattgtcacagcagttggagggcaaacatgtcattacaatcttgattggtcactgttccctccacccaaaacattcatttttcaataaaaaaatgataatattaaataaataaataaagctaattttccactatatgaaaaaaaaattgtggtgcgtaccgtttcagtagagtgtaagatttattattttaattgagtatacaaaagatcttgtttccaaatctgtcggtacaatttttttatacatatattcctattataatttatttatagctaaaattacattaaaatgcaatagtattgtgtgtatattgtagaaacaaaaagttaactatgatcttaatggcggcaaatataattatataaccacaataaacaagttacatactcgacaaatgtagaatgaaaaattaataaaaatgataaaattgaataagcaatattctacaaaataaatactactagttttgacttttgacttttgactcgttacattaaaaacctaaaacctattctctggcactaaaaacagattcgggtaccaccaactagagcagcctattttacaagaaatcacaaaattaattaatgtgataaactaaattactttgttttccgtcaattgataaactgatttaatgatgtttattgataaactaattatttcttcggtttggtgatacaacattactatttatgatttttgtttatctaatgttataaagaaagttattaaaagaaaaaaaacgtatcgtctcgctcgctctctttagatctgcttttcgtttttgtttgtcccatttataaaaaaatcaactggtgggagctaacgctcgcgtcttcttcctcttgacgttattggaattcgtagtcgttccgattcactgagccgactcggcgtagtagtagtagtttactactggtgatgtgttgttctagctgtttgtgatgatcaatttctctacattggactgaagaagagagtagtgttgataaatggctggttctcagcgacaatgctcggagctccggtgagaaatcctgtttctacggcgatcaagcacactcgacggtggagatgtttaagtcatcgagatggcggagcgagaagagcaacaaaatcaaaatcgttttcaagttgcagtttcatgcgactcaggtaatttcactctcggagaattttccgattcccggtcaaatttttttctgatgattttctcgggaaaatgttgcttagattttgaatttccagaaattgggggaaaaattactcaaatctgacatctgatgtgacctgtataaacagtgaattccattttccataattcggattttggaatttagatcttggagatgtgagatcatgttcatcattatgtgattgattgattttttttgtttttgtttgtttgtcaggtgacgcagttgaaggcggagggattaacaatctctgtagttcccggagatgttgggaagtcgacggggaaggcagagaaggctatggtgctcgacggacattgtcggtgggaatctccggtgtatgagacggtgaagtttcttcaggatgtgaaaacagggaaggttaatcagagaatctatcatctagttatgtcaacgacggtgaggaaaattaaactttctcagagttatgaagagatttctgagattacttttctgattagttttgtttttgcagggatctacaaaatctggtgttgtaggagagacttcgattgattttgctgattatgtagatgcaattaagacttgcaatgtttctcttcctctccagaattcaaattcaaaagcaatgttgcacgttagctctctctttctttctctttctttgcattaatgtttgtcatgagacattaagagtgtttgcatccatagcaaagctcacatgattcttgcttctttgtctctttgaactgtgatactgagatttgaactgggttgtttcattatcaggtagcaatacaaaggcagttagaaaatgcagacccacaaaggtataggaaatggtttaagctcttgcaagtctttattcttttagtgttcttattgggagtctctgttttgtctgtgtgctctacagagtggtgaaagaaagtgatagcttggtgaaaaggtcacggggtcaagatttgaaatcccatttgagtattgaagctgatgaaagtcataaaagtgattcacaagaggtataatcgcttttgccatattcttaatttgtgaaaccattgtgagaaattctatttgtgtctgctcttataggagggtccatttggtaaagcttccaggattactgaattgaggcgtcgagcatcaatagaatctgatagtacattatcaagctttgacagtgtctctgaactggatacactgggagaagttgaaatcagaggagatcacattcagcaaaaccactcaacaatgcatcaccattcggttagaaatgtctatgaagagcctcatatatcagaatccgagtggtcaggaagttctgatcaaggaatcagcactgatgactccatgaatagttcaaatgatacaatcccgagagatacaacgaggacttcctcggataacgaggtagacaagcttaaagctgagctcggtgctttggcaaggcgaacggatctttcagaactagagctacaaagcctgcggaaacaaattgttaaagagaccaaaagaagtcaggatctcctgagggaagtaactagcctaaagcaggagagggacttgctgaaggcagataatgaaagtaataaagcatctgacaagcgaaaggaagaggcaaaaattaggaacaagttgcagcttgaaggaagggatccacatgttcttttagaagaaacccgtgaagaactggattatgagaaggatctgaattcgaatctacggttacagcttcagaagacacaagaatcaaacactgagttgatccttgctgtgcaagatctagaagcaatggaggggcaaagaactaagaaaacagttgatcttcctggaccaagaacctgcgagaggaacaccgaagaatcaaggagaatgtcctgcacaagtgagacagatgacgatgaggatcaaaaggcactagatgagcttgtgaagggacacatggatgcaaaagaagcacatgtcctggagcgaaggatcactgatctctacaatgagatagagatctataaacgagacaaagaggatcttgagatacaggtggagcagctttctctggattatgagatacttaagcaggaaaatcacgatatctcatacaagctagagcaaagccaagtgcaagaacagttgaagatgcaatatgaatgttcatcttctcttgtaaacgtgaatgagcttgaaaatcatgtagagagtctagaagctaaactcaagaagcaatacaaggagtgctctgagtctctgtaccgcattaaagaacttgaaacgcagatcaagggaatggaagaagaattagagaaacaagctcagatctttgagggagatattgaagctgtgacacgtgctaaagtggagcaagagcaaagagctattgaagccgaagaagccctgaggaagacgaggtggaaaaacgcaagtgtagccggcaaaatacaggacgagttcaagagaatctctgagcagatgtcttctacgttagcagcaaatgagaaggtgactatgaaagcaatgaccgaaacccgcgaactgcgcatgcagaagcgtcagctagaagaacttctcatgaacgctaacgatgaacttcgagtaaatagggtagagtatgaagcaaagctcaacgaactctcgggaaagacagatcttaaaacgaaggagatgaagagaatgtcagcggatctagagtatcaaaagaggcaaaaagaagatgttaatgcagatttaacacatgagatcacaaggcggaaggatgagattgagatcttgagacttgacttggaagagacgagaaaatcaagtatggaaaccgaggcatctttgtcagaggagttgcagcgaatcatagatgaaaaagaagcagtcattaccgctttaaaatcacaactagagaccgcaatagcgccatgtgataatctcaagcattctttatccaacaatgaatcagagatcgagaatctcagaaagcaagttgtacaagtgagaagcgagctggagaagaaagaagaagaaatggcaaatctagagaacagagaagcgtcagctgacaacataacaaaaaccgagcaaagaagcaatgaagatagaatcaaacagcttgaggtaatgtgtctaacactagaacattcagtatttggctaattgttttgatgttgatgccaaattttggtaatccagggacaaatcaaactaaaggaaaatgctttagaggcctcttcaaagattttcatcgaaaaggaaaaagatttgaagaacagaatcgaagagttgcagactaaactcaacgaagtaagtcaaaatagtcaagaggtaagagaattaacattttacaatttagatatacatttcctataacctcaaagatcggaacataactctggttttatgaacagactgatgaaactctgcaaggtccagaagcgattgctatgcaatatacggaagtattgcccttgagcaagaggtatgttcaagaactagtataaacatagaaaacaccaatgatttctgatagttttctacttttacagtgacaatcttcaagatttggtaaatgaagtagcatcgctaagagagcaaaatggattgatggaaactgaactgaaggagatgcaagagagatattcagagataagtctaagatttgcagaagttgaaggtgagagacaacaacttgtcatgactgtacgttatcttaaaaatgccaagaagcgttaaaaaccatattctcagagtcagaagaaccaaatggcgcaatattgtacaaatttctcttattttcgatatatagctttattttttgttttttcacatatatatagtttctccttttttatatatatttttttctgagagaaaagtgagtttcgagttttggcttttttttttttttttttttggtgtatgtactacttgtaatagataccctggatataattattgatgttagtttgcttattatattaaaaaaatattctttctaaattcaccatttcaatatttgtttcaaccactccaaataccaagagtgtaaccatgaaacaattttaaaatacaatcaaagtatgctcactagtttaactgttacaacagatcaatcaactcataaataatcaaataaagaatctaatctacaacaaacgtagcagatgaacaagaaagaactaagcagctttgtgaatgagcttctaaattaataagtgttacctctctcttcatagcaaagtcggatacttggcgtcaagaaactctctgagacttttagcaactttgtgaccacccatgagttctgctaatgtctcaacaggcaaagacgcgagctcagccaaactcttacatttctccataatcgatctgtaattcgcatccgaaactccaggaagccttctcagaaactcaactgcagatgtgttatagttctcggctctgtttcacatcatgagtcaatttcgatatttacaaaggcaaattaacaaaactgatttttgcagtttaagaaagagagttcctttacctgatgtcattttctatgataccttcttctgaaggcacaccaactcttattgctcgagtctcgtcaggctcgtcttggttggattttaaggtagtaaagatttctgcggttgcatgtagacttcgagaccacaataaccttaaccgaggaaaatgcagaactagcaatgatagttttgatatgatgttatatggagtcacatcatctgatatatcactcgaagactgcaaaagcaagaaagaaccgatccaaactcaatgaagaacagatcaaaacatgataagacgaaaaattattttgaccttggaatgtgagagggtacctgaaaggagaagcttttgtcttgtgagaactcgatcagaagaactggtattctgtaataacgggacatcatttcgacttggtgaaataaacgacctgatgtgaagctctggaaaagatcttgaatactctttctctctacgcatattgaaggagatagaatatagtcgccgacctctagtgtaactggtattatcttcatgcctttctggtgaagaacatttggtagactgctcatgaactccctcatgtcaactataacctggtgaaaaacacatttccataaagtgaactctttagcaaatctagtcaatcttgggaaaaaatatcgttcttgagatatgaacctgtgtctctttctccagttcctttcttccgcctgcttttcgggttaatgagttttgtgtacttgaagctggaaactctgtagaagaattcgaccccatgcagagcccatcctaattccaagtttatagcacagtatggatgagaagaaagcaggaaaaagaaactgaaatccagaagataacgtaaaattctggttaatagtttcatctacttggtctgattcagtttaataatattaacttgttttagcaacgctatcactaattgagttgttctaatgtactttcaaatcaaaactgtatactttaaagcaggaaacaaaagtcacacaggcttcgtttcatttgaaatgttgggaattgagtttcaaacctgatcaacgggaataatcatcgaagacttttgcctgatcaatgattcaaacgcttcattttctcgacgtatgcttgcttcaaacttctgcacttctgtggactcatcatagaaaataaaataaactttcagctttctgagagggttctctgctttgtaaacctcaagttctctgacaaaacccatgtcaggatggtagacaatgatcacagagggtttcaagatatctagtataggttgatcgctttctaaggcataaaaatgcacaggcggtagctttttagcaccgctggggcgaacagcattggcttcttgaggggctgacgtgctagcttcattggctttttcttcattctctgggcctggtaagatttcaggtttactgttcgtcgttttcttattgcaacttcttaaggatctccggagacttgttggatcctttttagcttttcctttggtacacgaagccttatcaacatgaggctctgggttgttatttccagaagccatatctgttgtctttcctagcttgcgaatagaagacgcagcagccataagagcctcatgctcttgtctgccaacactgctaccttccgagttctgtattgtagttaccggaacaactccatcaagaatcccaaaacccttgggagtcttttgtttcttcttttgcggtgtctgcatactgcggagctcaattttgctcagcaggtacatttcccattcttcccgcattacctgcacacgtgtaggtaaaatgggaacttgaatgctagtttctagtgacagggtacatatcggttgttaatgaatgatgtaagatctagcaaaacggagttatctgactggaccttctgtggattgtttgtgatgcaatcttcaagctgcatgcaggaccgttcatctttgcaagccacaagaactattccattgttgtcactgttatcttcctcagaaaaagcctgctttagtctttcttcttgtgtctcctctaaaatttcctgtattaatcacaggcataccattacgtttaaggctaatccatgaaaacatagagtgtggcgaccaaatcaaagaggtaaacaacaaagaacagatatggtcactagctaaaccaacttccatcatcaaattaacagtaacagagatacggcattgaaactgaaactcactcagtcactcacacgtaagactttccattttggtgcctcttccagaacttcttctacaacaacgccagtagccacatttgttgccgtttccccaccaactgattcaagtccaattatcaggaaatttttcgaatgcttataacacacatgtcactagatgcaaaatacaaccattatagatcaagaattagttacctgcttctacagagtcagtttcgcccttcgaatttctctttttaccactcttattcttaacatgttcttttgacttgacatcacttgccttcacgagacgatacactcgtttctttgcaaaatcaaatattttatagctggactctgcaaataaccaaacagaccgatagctctctgatacccttagtgtatccaagaacttgagaaaactcacagcatcatacctacacagaaataccaacttgaaggttttgcataagaacattactgatctacagagaagagagacagagctattattacctaacaagataatcaagcaacttccttaaagtcttcaaatcagaaacaagctgttttgtccttttccccaaagtatgccaaataggatcaagctgcctcctcacaatctcatcaaaggacttaaacaaaccactctctacagtcaaatcatcaacatcaactttattagtcttcttcatctccttgagacaagcatccattacttcaatgatagctttctgtattcccaccatgtaattcgacatcgaaaccctaatatccacaacctcaggtggttctcgctccaattcctgagacacatccaactgaaaccttggccatagatggattttacggaggaagagagcacgcatggtgcgttcagtcttggcgaaaccggaaaccatagcttgcggtctatcggaaaaagcgcgtatataagcagagctatttagggatttgacaattcgtataatgaatgcttcagtggaggtttcggaaatcgaatgagcattgaggatgaagattccggcgagtgaagaaactggaatccgttgagtcaacagatcgacgatgagaatccgcggcgtgatgaagaaaggagaaccggaggtgtaaagcgagtaacgctgattcgccggaagatctgcggtgatttccgtcggagtaggagaatcaagagaggagatgtaatggatgattctggattttagagattgagcggcgggggagaggaggagaagtagagttccttgcgacggagagtgaaggatgaggagagaagcgattaatttagcgagggagagaccggaggaaaggattaataatccgccattagagtcctcgagaaggtctgatatgatctgttggtgatatttcagcgccattgaagaagacgacgacgcaaatcggagaaactgattgatgaagagtgaagacgatagaaaaaacctttcctgggtcaaaaacgttcaaacttcaaaacggcgtcgtttaaataaagtatttttcgaaaccggtttcgatatttcctatattaattctttcttaaccggtttaggttagctaagaaaaagaagaaaggtcctgagacttgtcttataatcctcaaccagacttgatccattagtcaactcttctcttcacaatgggtaaatctctctaattttcctcatttcaatttagattctagaaatgtagtgaattgtgtaatgatcaaaacccaattcccagattcttatcaaagttttcataaaagggacaaaatttcttgaaagatgtgaacttttatgtgttgtttttgttggtaacagatgaagaagaagcaatgttgctgcttaaggaagaagatgaaggaagacgaagaactagtgttcctacacaactcatgaaactaaatagaagccaatggtggattcttgttttcataagcatcttctttctcatctctgctcaagccatttctgttcttcttggtcggttttattacaatgaaggtggaaacagtaaatggatctctactcttgttcaaactggtggctttcccattctttatctccctctttctctccttcctgcttcacaatcttcttcttcttcttcttcttcttcttctttcaagactctggtttggatttatctttctcttggttttgctattggtttagataattttttatactctgttggacttttgtatctctctgcttcaacttattcgattctatgtgcttcacagttagctttcaatggtgtcttctattattacatcaattctcagaaaatcacttgtttgattttcttctcagtgttgtttctctctatctctgctgtgttggtttctcttgacgatgattcaaatagcccatcaggagattctaagtggagttacttgattgggtgtttctgtgcagtttttgcttctcttatctattctcttcagctctctcttatgcagttttctttcgagaaggttctcaagagtgagactctctctatggttctcgagatgcaaatctatacgtcgcttgtggcttcttgtgtagcggttatcggattgttcgcaagcggggaatggatgttgttgagtgtggagatggaagagtttcaggaaggtcaagtcatttatgttttgactttggtcggggcagcggtttcgtgtcaattgggttgtgtaggagccgtgtcgcttatatttctggtgtcttcgctgttttcaaaccttattagtacgctctcactcattgttacgcctctcgcggccattgcggtgttccatgacaagctgactgaggttaagatggtggcgatgcccatcgccttcacaggattcacgttttatatctaccagaactatcttgatgacttgaaagtacaaagagcacgagaagctcaggccgaatgatttattaaactctactaaaaaagctctaaagattgacattgagtaatatttctcggttgcatgtctccaaaatatacttaaatttgtcgttacgtaatacagttcaatctttgtgcactgtctaaaggaacctatactgttcaataacttatgaatttgtaacttacaaattgtggtctaccaactcatttgcttctctgcacattctttacgaagtctttttgttcaatacactgtctggaataaacactattcaattagtcattcttcattttcactaacaaaacaacctcacagcttcacgaaaccctcgcttcgcaaagaccgtgtaaactgctactacaccatcattctctgtaagttgaagaagcatcatcagagaatcctcaacactatgatcagttatgctatggtgtcacctaattatgaaaccccatcttctttcattttcctaaacaacacatgagcttcatgctttaacccttccctaaacaatcctgatatcattgtagtatatgttacaacattaggcttcactccttttgaaggaaggctacaaaataaattaacagcatttttcactttaccagccttgcacatcccttgaatgatgatggtatatgtaataattccaatatccatctctctctttcgcatatcctcgaataccatcaaggctttctctagcttcccattataacatagaccatgtaacaagacgttgtaagtccaaatattgggagcgacaccatccatttctctcttttgcatatcctcaaatatcatcaacgctttctttaccttcccgttataacatagacaatgtaacaaaacattataggtcctaatattgggaggcacgccacgagaaaccatatgactaaaaacttcttgggcgacattaggtttgcccacttgaccaaaaccttggataagagtagtgtaagtgatagtgttcccagtcaatcctttttgggacatctcgtagaagattttcatcgcatcatctaccttcttacacttgcaaaacccgtttatgagagaagtataagccactacatctggaaaacaacccttggtttccatcaaataaaacatttgtctggcctcatctacacaaccttccatgcaaaacccattgatcaatgaagtataggtgaaaatattaggagctatagacatacgaatcatctcattgtacaattcttcagcatccaaaaactttccttctttcacaaacgcatcgatcaatgcattgaaagtgattacatcaggtttgattttcctcttcgtcatacccctcagcaatgaatcagcatctctccatctaccagagttacaaagaccgttcacgagagaggtgtacataacaacatccggtctaatcccgtagttttccatttgatcgaaaaggctcaacgcgtaattcacatgcccgtttttgcaaagactatcaatgattgttgtatacattacaacatcaggtttaatccccatctccaccatctgattcaccatagacatagcctcctccattctattcccgagacagaacccattgatcagagaagtaaacgtgacaatatcaggctcaaaaccaagtttcatcatcttcccaagaaaagatgaggcaagataaggctgagaagattggcagaaacaattcatcaaaagattgcaagtatagagatcatgtgaaactcccattatctgcagatggtcgcagagattgatcacaacatcaaacttcttcattttggcaataacattcaataacttagtgaaatcgataattgaaggaagaggacgagactcaaccatgtgagtgaacaaatcgagagcttcattaaactgaagagagtgaagaccatttctcaaaatctctcggtaattacaaaaagctcgaacccagaaactaagatctaaaaggcggctaaaggagagagcttttccagaattacctttcacaagacgatttcggtgaagttgaaaaaatctcatcgccattaacgcaaatcccagacgaaattgattatgccgttgatcgatttactacaaaagctacaaaaaagtttggatgagatcgaaggtcgttgtatttaagaagcctaattttcgtttacttggaagatacgaagaagatgaaatttgagatttgagtaaagaagcggcggaagagaagatggcgaatatttgtacacgtgtattggtttgacttttgtataatatcgttgacttcacgtaaccagtctacaacgaccggttaactaaatcggtttgttcatttacagatccctagaccaagacttcatgttatgcctgattgcgatcacttgcagaagaattcctcctattattgaaaggaagtataaattgaaattttgtctaatttggtctcaattttgattttagttttggattgtgttctttgctattcaaaattcttaagagtaatttggtctcaacaatatcttaaactttatatgatccgatttaatccaaaccgttcaataaaattaatttctatcaatctaaaccgttcaacaaaaataagtaaataacaaccaaaaaaaaattaaacacattttaacacattcctcttatcactagataacaaaaaaaaaatctcaattattatttagttatgtttgattaaaaaatttatatttataagtttagtatatcaaatttattcaaatatttagtataaaaactaaataaaccgattgtacgcggtaaactgcgggtataattttagtataaaaccaaaaatgaaaggaaagatcttcacaataattaaactctatggcttaagaatcttattaaacgctaattccgtaagaattatatgaaacgctaattcggtaagagtttagtaaaccagattgtaacaaacgtgtaaatatttttctggttatgaattttttaatgaaccggattaaaccaggaattaaactgtgcgtttaattaaatatgaatttaagtactcaatcagcaaattggtattgactcaacatgatggacataagaggtttgtgacttctcataagagctttgtattttattgatttgaacacttttcttagtagtaaattaaattcatggtcgaatctttgtttatatatgagtagttttatcacatagccgtaccttgcctttggttggttttgttctgcttttctaacgaaccatattaaaaccatgtatttagttatatattaaaaaggtttaagtataattaaatagcaagaagattaccaaatccggcccaaaaaaatggtagtgactcagcttgatcagagagtctcaccaggattcgaatagggatattgatattcttaacttaattataaattatatttaggacaaagaactatatcttaaatcttaatagaggtggagctatggagtgacttttgggcgactcaatcaactaggttctcgagtgagatctcttttcacaaagagcccttatcaacataacccaagactttaggtgaaagtaacgcgtaccacaaacaccggtgaaaagaaacctatatcttaagggtttgtgacgtgaatatgatgaatccaatggagcatcctcatagaggatttaatcaatcagcattgatcacgccagtacacttagacgtgatgcacctcatggatagaaggttggtcttatcgctgctaggaggactggtcgtctcaaagtcaaaggtcaagttgttgcttcagccgccaaggtcgacaagggttcttaaacataaactcttttactatttttttaaaggttttaactagactatccatagtagctcaagaaagagagatagaaacatggaacttttgcaaattgtagtcccaaatatcttttattttaatatttgtcttttatccaaaatatgaatttgtaattctctgttaggaaattgatttgcagtcatataatttacatgaattttttaattatatgttacgaaatatacattttttctgtatgtgttgcacacaatttttttaaaaatatatgaatactttaattttgttgatatataagcgatgttatgaatcaccaggaataaatataattggatacaacatttagacatattgtaataattcatttagaaacaaaatcgaatcccagactgactgtaagaaaataaacatttgatcaactattaaactaaatgattttcataatcattacatagatttaaaacagtaaagagttacaaatatttggtgataaattggtaacaaatagtctattcatttgtgttaatgttattgttgtaacataattaaaaccgcatattacatgttattaaaaataacaaatatacttttaatagatagtgatatttaaaaataagaaagaaatctagagtagttacagtagggccttttttatatattctaatcctaatttacttttttttttgtcaaacaatcctaatttactttattaaactaaatgataaagtttaagaaaaggtgcaaaaataataattaaaatattaaaaataaaataaagaagaaaatataattcttgtgatttgtgttaacctttcttttatttgtttgccaacaatgattgtaaattctaattaataaaaaggaggctatgaggaatataaccacttttgaaaatctgtttttttcctctataatgaatgatgagttagacttttattaaacaaagttgaaatcgccgacaaaaaacaaacaaaacgttaaatctttcatcagttgagttaatgtgatatggaaatttttcaaatagtttaatggtctaaaatgatattttaattattatatatatcgtctataattcataatatagacataaatcaaattgaaatctacattacgacatagctacaacactaacatttttttatccaacacttaaaatttattggaaacgtatatattttgtttttatttaatgtaataatattttgtcttctttcacattttaagcaggttatatattgactataaatgtttcacagatagatgcatgttgatacatttttccttgtatacaaaatacacattacagttaaataaatttatttatttctggcttacaattagagatattactgtgaagtgtgaacatgcattagatgggaaagaaatataaaacaatttcattacataaaattgggatctattactaattaaatgtggaataatcttaattttagtcaaagttatagggacacatatttaaataaaagtgatatctttcttttctaaaagacaaaattgaaaagcaaaatgtcttcttctccgtttagaatagaacaacaacaaaaaaaaaactgtctttgaatccaagtctctctcttttgtcaccatctctgttacttactaagaaacttctttttctttaatggtttttttgctaaatacccgtaatattattaattaaagcattttcctttttctgctaaatcttgctttgctctttaagctcttgtcattgttgttaattgtctcctggaggctggaggctggagattatttggtcttttgtgatgactataatgtgagaaattctgggttttgctagaatttgaagaaatctttgagcaaggaggaaaaaagaatgagactttacttatcttcaacgatgcagctttctcttatgagtcttgttctagggttcctctttgtttcctgtgacgcgtttgcctctaaagaaggtattttgatttctccattttctccaatttttggatgctgagaaagtttagtctttttagcctctgtctgttaacacttgctcattgagttgatctagaaagttagaaactttagttttgttactgatcatttagaagtatttgatgttttgctgttttgtattcagttgaagcagttagaagattcaaggaagccatttataaggacccattgctagttatgtctaattggaatgtccccaatttgagtccttgtgattggaatggcattaaatgttctccatctaaggatcacattatcaagatgtaggaaactttgatctctttctatcagtaaaatcagttatgtttagtatgatgatgatttggtatctgtttcatgctgtgaaacttgcagaaatatatcggggacatcgatgagagggtttcttgtgccagaacttggtcaaataacctacttgcaagaactgtatggttttgattcatattgacaatacctgaagatataagtttgatgattggtactgtttgtaaatgtttagatgactttgttttttctgtgttgaatgcttctttaggatcctgcgtgggaacattctaatggggacaataccaaaggagataggaaagttaaagaaactcaagatcttagacctgggaaacaatcatttgacaggaccgattccagcagagatcgggaaattgtcaaggattaagacaatgtaagaaaatctttaagagaatgtcatctatccgataatgtgctgagataaccattttgtgtctctttaacaccacagaaaccttcagtccaatggtttaataggaaagttacctccagagattggaaacttgaagcaccttaaagaacttcttattggcaggaataggcttcgaggaagtattcctattgccgcgaaaacatcaaaaaagtgagtttagctaatagtccaaggtagcataagatggaaacttaatgtttatgattgaaatgttaatgtatcttctttttgtgttggtcaggtatgcttcaaatccaagtgcaaacatcagtggtttgtgcaagtcttctctatttaaagtggcagatttctcttacaactttttcgagggaagagttccgagttgcttggattacctcccaatgtatttcttataagaccctttttctagctttcctttatttttctcatttgataatatctctctgtatcattgaacatcattgtagaacgagctttcaaggaaactgcatgaaaaccatggatgttaagcagagacctctttcagaatgtggtttgtagaatatgagtttcactttcttgatgctgataatcgtttctttatcttgtttttcattttgaaattgtttcaattggttagctcgcttagctgtaaccgtggccaagaagaagcatcgagcatcgagacaaacatggcttcggaattttgagatagtcacgggatcatcagttggcttgctctttctagtcgtaatgttctctgcatgtagcttgtgcaaaataaagcgctctctcatcgttccctggaagaaatctgcaagtgaaaaggagaagttcacggtctacgttggttagaaactcttaaaaattctaagatttcaatacaaataactgaaagagcttccagagatgaaaaaattactgataaactgtttttctacagattctgaaatgctgaaggatgtttcaagatatacaagacaagagctagaagtagcatgtgaagacttcagcaacatcattgattctagtgcagagagtcagatttacaaaggaacgatcaaaggcgggactgagatcgcggttatctctctctgcgttaaagaagaaaattggactggatatcttgagcttaatttccagagagaggttcttcttcttatggttgtttatcaccaagtcacttgcaagaaaacatcagtattaaacttgattttattaatattcattgtttcaggttgcggctttggctagattaaaccatgagaatgcggggaaattactgggatactgtaaagagagtacaccgttcacaagaatgcttgtgtttgagtatgcatcaaacgggacactatacgaccatctccactgtaatatataatcaaacttcttcagagctctttctttggtaggactgataatgataccaaatgatgataaaaatttgatgcagatgcggacgggagtttagtatcgtgggcaaaacgcatgaaaattgttataggcatcgcacgtggtctcaagtaccttcatactgaactccatcctccatttacagtctctgagttgagctcaactgcagtgtatctcactgaagattttactcccaaagtaaatttgatcctcttttttctatgcggttagctaagctttgctactatctctatactcttattttgatcctcttatttctatgcagctggttgatttcgaatgctggaagattattcaggtgagatcagagaagaacctgaagaatatctgtaatgaaggagcaatatgtgtacttcccaatgcaatggaacaccgagatatggatttacaagggaatatctactcatttggcatacttttgctggaaattgtaagcggaagaccttcttattgccaagacagaggttgcttggttgaatgggtaagaatagcttttctctataagcttaaagctgagtacttataataagtctctctctctctcttagcggtttacgtgttttcatgcgtttgatgctgaggatttgattcaaactcctaaatattgcaggtaagggagaaaaaccttggtgcaccagatgtgatggctagcttggtggatcctgagctcaagcatttcaagcaaaaagaacttgaggcagtatgtgaagtggcaagccaatgtctgaacttggaccagaatgaaaaagacaaggataagctttcttgttcgattcaagcgctttgtgagacactagagagtagaatcactgtgtccatttctgcagaattcaaatcgtcttctctggcgtgggccgagctagcgctggcctcgccttctaacgaagacgacgatgataggagtaaataaaattggctttgttgttatgacattgacatacacttgttcgaacatttttgcttcaattttgcattcggttttgatagcaccgaccatgccttggataagttatcaggtaggttgttacggtcggtctattgttaataccaataaactggaggtgtaatcttgtataccaagttcttgacgaatgaaattgtgttgagccaaaaaagaaaaaaatgacaggtagcttgaaactagaggaatacatctgtgaaccgaataaaaagttataaactctcaccttttcaaactagttttggacttcaaacaacaatcagaaagaaaaagtaaaagtacaaaaaagagacaaaatcgttggcatctgaagtacaggagatgtttgttgtgtaggagaaacaaagatcagtgcatagaacgagagtaatggttgtcattagggtttcttttctggctgaaaaccgacatagcttttctcacaggaggctgcaatggcgcctttctatcactatgatggttcaagaacacatcatctgatacgaagtactcatcaccctgcaaacaacagcataatttaccttaagatttcaatcaaagttacagatttgagtttgagatagaagacacgaagagactaaccggtttgcttgctttcttcttggtcctagggtggagaagcttttgagggagttgatcttcccgtagaatcagattcttggtgattgcctctcttcctcctttaggcattggtatcgaatactgcacagaaagaaaagacacatgtatcaggagaagtttttatctgtgtattaggttgcagattttaaaggaaacaagggtttaagtttaaaggcagtaacaaatgacaaattgatcatatacatgccggaaaacatggtggttctgttctcatgctatttcaaataaagaatacaagattcttcactaatacaggaatttttggtgttttggttagcttctgcttataacaagttacttacttgggtccctcgaagtgtaattcgtggtttacatgcagctatgatggcgccattcgcgcctattgtaaccgctaccttgcgtagagtgccaccattcccacatttgggacagaagattcttccaatttcaggagttactgtgtagcaagcatggcattttaagatccacctgaagatgtcaatgcaataaagtgtaagaattgtatctttttcagttaatgacccttgctaaagaaaagtgtctgacatttgaacaaaaaaaaggaagatctgatcaatttagagtacctatgcagttggcgaatttgcattccgccgggtgcaagcaatcgcaaacccatttgaagaataacattctgcattgcatagtcaccagttatacaagctacactggactcagataaagctctcaatgaccaactctgctcgctgctaccatcatctccaatcgatgatgcctcgaaggtatcctcagcctcactagcaatctccaagttctctagcgcttgatttgcaacatcgatcccctcagcttcgacttcaatatcatgatcaatatcatcctcaccattgatcaaagttgcttcagcattcgtctcttcagtctcttcttggagagcccttaatgactcttcctccaacctcatatctttaaggatggaagatatatcctcaccattctttccactatcttttgcgtgggtttcattggcctctttagtgtgtcgagctttgtcagcttcttgatctttctgaatttcttgttcagccaaagcattgtaatgttcccaccttgcctttcttctaagatactttgagtgagtacttctactgactgcgggtctccaatcactagcatcatcatcatcatcatattgcccctgggatgcatcaactccctccactaccatcttaccctcaagcttaatctctgttttcttgggcgggtatcgcctatgcttcttcccaccttcctgcacatcctcttcatgattctcagtatgagagacaacagaaccaacctcagaaacattgtctgaagcaataatattcatgttaaggtctttcaatggaaggattttggagttagcgttggatttttcctcagtctcgttttcaagtgcctcccactcctccaagttagccacattggagccccaaccaggtaaatccttctcaggtaatctcttaactctaacagtttgaatcggtggaggaacatctctaagatttttggtgccataaacctgagcctcaagcgtgtatgacaaagcaatcaacttcagatcaacatcagacagagattgcaaatcaccagtagctcttgcaaatttgataactgcagaaccaaaaacaatcaatctcagagatacaatacaatcttttctccaacacaagaatatacaaatacctacttaggtacaaatttcacaatcacaagaataagaatcacaaaactaataagtgtacctttacttagagattcaggagaaggttccatggtatcaatagtgaaagggatgaaagcgagacgacggcgagaagcagggtcacgaatctcagagagaacctcagggacagttacgaatttgtcggcgaagtttgttaagctctgacgtccttcaatgatggcgttggcgtcaacaaccgcaatagatattccttttgtggatttgcagtttccgaccatcccgagaatcgctgccggagctccgtcgttaaccggaggctttgaaggtggatctttcttcacaattgagctccacatcgaggttggtttcggatccatggttttcgttatttgccgcttcaaatgtgggtgggtttaacgaagagactgagagatggagagaagaagaagagtgtaaacaaaaacctaggtcagatccgagacgacgtcgctttttgtccataattttaaatgggcctcacaaggcccaaataattgcgtagatgtataaaatactatctgattagagctagaatttgaagactagtattaaccaaaccaaactagatcatatcaagggttaatgatatcaaattatagttgtgtttcctttttatcaacccttaacggctctagaacgtaaaatctatggaaatcgaatctatgcaaatatatggaaatcgaaacgaatctatctatgcacacatctttacgcgtttttttatcaccgatcatctataaatatatctaattctttctaactatttcatagttcatcatctttttcacaaagccaaagtttgtcttcgtgctttctcttaactttgagcgtgaaaaaacacagagtttgtctttgcaatcaacaagaagatgactatgcgatcatcttcaccttcgtcctcttcttcttattcactcgctttcacaagtttgagcaataggcttgagaccatcttcaagaaagcttcagagctttgcactctttgtgatatcgaagcctgcgttatctattacggaccagacggagaacttaagacatggcctaaggagaaagagaaagtgagagacattgctcttaggtatagtctattgaacgaagcattgagacgcaagaaaagcgttaatcttcatgggttcctgaacaagaagaagaacaagggtttgaagaatccgaacaagaagatgaagacgagtcttaagaacgtgaatattttaaagtatccactcgctgatcattaccctcccgaccaagtttctccactgattcagtccttggaactccatgtctctaaattccaagaaaggcttcgatttcttgagtcgcagaaacagaatcagacaaaaccggatcatcagagtttaacaccatcctctctgaatcattacacccaatctttgaaccctagccagttctcgctgtttatgtataaccatggagacaatactctgtctcagatcccagtctctgcatcaaatttcaatcaggattatttctcagcgttacttgaagaatctgagttgaagaatcagttaatgaagccggagatttgtggttatgatcagaatcagaacatgagcatgggtgacatcacaaacaacaaatttcaagatccttgcgtctcaaacaaagaagcggtacaagaatctgtcaacaactttgggttgaatcaattgatgtacaaggagttttatggttgtgatcaaaacatgtctatgggtaacatcaatagcaacagctttcaaaatccttgcgtctcaaacacacaacattactcggcggtagaagaatctgtgaagaacccctggttgaatcagttaatgcagaatgaactttacggttacggttatgcaggcttttgttaaattcttatgatttattaagaacatcttgaatcaatttgatgtgttgttgttgatctgtattcagctattgtaataaatttgaatcatcatccctagaaacattaattactaaaagccaattttatctatttctgattgctctctatcataataaagccaaatttccaatagactctcaacagagcaataaccgtggctgagttctgattcctccactttgtctttgtcttttctattcacagatataacatcctctcttttcaaacctgtttcagtgattctctgttctaagaaataataatcatcgcatgtttattaaacggaacaacataagcattacagtttcagtaaacatgagaacagtgtgtgggggtggggtgggggggggggtgggggtggtgtttggacactaatgtttgcaagcttaaagataaattgtgcaggtgactcttcacactactgttattttttgtcttgtgatgatgattccttccctaccataatgtctgcactctgcagcaacttccctctcctcttgacctttatcttgtgatagtccctccccttccattatgtcagctgcagtttcccacccctcttggcctctatctcctgatggttccataagtttccatacttccattatatttccaccttgctttcctttgagttttagcagtgcagctacccacttttctcgacctttaccagctaaaaacgatcctaacagtctaaggcccaaaggaagtaagccagtgagatggacagcacaaactgcgagctggttcaactataatttacatataaatctaaaccttcatgttgtgatgttcaaaatgctcgggttcggtaaaaacctttgataaagtgattattacatatagttcaaaaaacaagtgattattacattccttatttttcttacaatggagaataggagataatcggcttttcttcaacgaaccagtgactgacccctgaaaccggttttgccaaagttttgttctaagttcttagatcttggattgaagacccggtttagataaggttcccatctctcgccgcttctggaatccttctttgactttgaaaaggatctcatgggtctcatcaaagtgaggcattgttgcttttttcgtattctcaatccattgttcaactttcttatgagtgctgagcaatctaagacgatctttgtcatccaaaacctacaaaattgcataaattttacaatcaaaagccaaaacatagcaaggattggtatttcgatgaattaaaaaaacgcacaaaacagtgaaattaagtgagtacctgcagctgcataagttcgcatacaaggctgagatcagctatagatggttggttgcttccgagcaagaatttagcattgcccttaagccaaaaagtctctagagtggacagagactttgttagtaactgctcagcttcagcagctgctttcggattcagaggaagaccgagagctggacctagtacactattcagaacatatccagctgcaacagtaaaaacaaccgtaatcagaactatttgactctataacttctcaacatcgatgaatataatgtacaatacctgcaccacgacgtaaattggtgtgatgccaatccaaaactgagtgaatcttggctcttttggatagatcatttggatacctaaaagtaaacaaatgaacatcaaagcaaaatgattacactttgtgtcaaacgaagtagaaacttaccaatgatcagcaacacttggaaacgccgaggatagatatatcaggattgcgtgactacgaaaagaatcaacggttaagattcataaaaaatggtcaataaaccaaagcaagaacccaaaagagaatgaacttttgtgttaacctctcaaagagcttaagcctgccatcaacaatagctggaactttccccaatgggttaatatctgaagaacaagacacatttcttaacaatatttacattaagaaacaacactaagcatatatagccttagtctacactaatctacactaatgatctcatatacactaaacataaatctaccaatactcagatttgaaactttgttgattatttgtacgaacctttgaattcaggagataattgttgacgcttcgccaaggaaatcaagacctcatcgaattggattccattaaccctgcaaaggataaaatccaaacaaaacaaagattctcatgtattagagaaaacaaaattggctgaatctggaattaacaaggaagttcaacgaaattacttgcagaatatgataacagcacgtgatggctgtgacattcgatctgcatacactttgagcttcatcatcttctttttcgtctgattttgtggatc

SEQ ID NO: 10 - protein, 670 aa, Arabidopsis sp.
METEIPRSTEISETLLLPETNLDHGEYVPEWKEQITIRGLISSALLGILFCIITHKLNLTIGIIPSLNVAAGLLGFFFIKSWTGFLSKLGFLSKPFTKQENTVIQTCVVSCYGLAYSGGFGSYLIAMDERTYKLIGSDYPGNNPEDVINPGLWWMTGFLFVVSFLGLFCLVPLRKVMILDYKLTYPSGTATAMLINSFHNNTGAELAGKQVKCLGKYLSLSLVWSCFKWFFSGIGGACGFDHFPTLGLTLFKNTFYFDFSPTFIGCGMICPHLVNCSVLLGAIISWGFLWPFISQHAGDWYPADLKANDFKGLYGYKVFIAISIILGDGLYNLIKIIVVTVKEICNKSSRQHNLPVFTDILDKSKTSVLMREKKKRDIIFLKDRIPLEFAVSGYVGLAAISTAIIPLIFPPLKWYFVLCSYLVAPGLAFCNSYGAGLTDMSMPSTYGKTGLFIVASIVGNNGGVIAGLAACGIMMSIVSTAADLMQDFKTGYLTLSSAKSMFVTQLLGTAMGCIIAPLTFWLFWTAFDIGDPDGLYKAPYAVIYREMAILGVEGFAKLPKHCLALCCGFFIAALIVNLIRDMTPPKISKLIPLPMAMAGPFYIGAYFAIDMFVGTVIMLVWERMNKKDADDYSGAVASGLICGDGIWTIPSAILSILRINPPICMYFRPS

SEQ ID NO: 11 - DNA, 2337 bp, Arabidopsis sp.
  CDS (80)..(2224)
tctcgttccaataatacttcctccaccatctctcctcctctcgttagatctaagaaacagagaaaacaagagagatagaatgagaaagggagttctaaatcctgacagagatcgtcagatagtggaacatgagttgcaggaaactgggtttagtccagaaacagagaaagtcaagaacaagaattttgaagaagatgaagaggaagaagatgaatctgtggagaagatatttgagagtagagaagtaccttcttggaagaagcagttgacggtgagggcttttgtggtgagctttatgctaagcatcttgtttagtttcattgttatgaagcttaacctcacaacgggaatcatcccttcgctcaatgtctctgctggtcttttgggtttcttctttgtcaagacatggactaagatgctccataggtctggtctcttgaaacagccatttactcgccaggagaatactgttattcagacctgtgttgttgcctcttctggcattgccttcagcggaggttttgggacatacctctttggcatgagtgaacgaattgcgacccaatcaggagatgtatcccgtggcgtcaaggacccttctttgggttggattatcggtttcctctttgtcgtcagctttcttggcctcttctcagttgtccccctgcgaaagataatggtaatagacttcaaactaacatacccaagtggtactgcaacagctcatcttatcaacagctttcacacccctcaaggcgccaagctagccaagaaacaagtgagggtgttggggaaatttttctctttaagcttcttctggagtttcttccaatggttctttaccggaggagaaaattgtgggttctccaacttcccaacatttggactcaaagcttaccagtacaagttctactttgatttttcagcaacatatgttggtgttggaatgatatgtccgtatataatcaacatctctgtcctattgggaggaatcctctcttgggggataatgtggcccctcattgaaaccaaaaagggagattggttccctgataatgtcccatccagcagcatgcatggtctccaagcttacaaggtgtttatagctgttgctataatcctaggagatggcttatacaacttttgcaaggtgctgagccggactctttcaggattatttgtacagctccgaggccctactacatctatttcaagaacctccttcacacttgaagaagaccctcatgcttccccactaagcccaaagcaatcttatgatgaccaacgtcgtacaagattcttcctcaaagaccaaatccctacttggtttgctgttggaggttatatcacaatagctgcaacatctacagcgatactccctcacatgttccaccagctgagatggtattacattctggtcatctatatctgcgcgcctgtcttagctttctgtaacgcttatggagctggactcacagactggtccttggcttcaacttatggaaagttagccatattcacaattggagcttgggctggctctgagcacggtggtatgctggctggtctagcagcatgtggtgtcatgatgaacatagtctcgacagcttcggatctaacacaagacttcaagacaggctacctcactttatcatctccaaagtcaatgtttgtgagccaagtgattggaacagcaatgggttgtgtggtatctccttgcgtgttctggctattctacaaagcgtttgatgatttaggcctcccaaacactgaataccctgctccatttgctactgtatatcgaagcatggctaaactaggagtggaaggtgtcgcatctctaccgagagaatgtcttgttctatgctacgcgttcttcggtgtggcgattctcgtaaacatagtaaaagatagtctccatagcaattggggaaggttcattccacttcccatggcaatggctataccgtttttcttgggaccttacttcgcaattgacatgtgtgtgggaagtttgatactttttatctgggaaagagtagatgcagccaaggctgaagcttttgggacagcggtggcttctggtttgatatgcggagatggcatttggtctttgccgagctccgtgctcgctatagccggagttaatcctcctgtttgcatgaagtttctctcttctgcaaccaattcaaaggtcgacaacttcctgaaaggatccatttaaaactcaaataagtaacaacatctcaaccatgtgaaagtgtaatgatgcttcaattgttcttttaccattatgacgattttgaatgtaactcgtatataaagatcttagatatg

SEQ ID NO: 12 - protein, 714 aa, Arabidopsis sp.
MRKGVLNPDRDRQIVEHELQETGFSPETEKVKNKNFEEDEEEEDESVEKIFESREVPSWKKQLTVRAFVVSFMLSILFSFIVMKLNLTTGIIPSLNVSAGLLGFFFVKTWTKMLHRSGLLKQPFTRQENTVIQTCVVASSGIAFSGGFGTYLFGMSERIATQSGDVSRGVKDPSLGWIIGFLFVVSFLGLFSVVPLRKIMVIDFKLTYPSGTATAHLINSFHTPQGAKLAKKQVRVLGKFFSLSFFWSFFQWFFTGGENCGFSNFPTFGLKAYQYKFYFDFSATYVGVGMICPYIINISVLLGGILSWGIMWPLIETKKGDWFPDNVPSSSMHGLQAYKVFIAVAIILGDGLYNFCKVLSRTLSGLFVQLRGPTTSISRTSFTLEEDPHASPLSPKQSYDDQRRTRFFLKDQIPTWFAVGGYITIAATSTAILPHMFHQLRWYYILVIYICAPVLAFCNAYGAGLTDWSLASTYGKLAIFTIGAWAGSEHGGMLAGLAACGVMMNIVSTASDLTQDFKTGYLTLSSPKSMFVSQVIGTAMGCVVSPCVFWLFYKAFDDLGLPNTEYPAPFATVYRSMAKLGVEGVASLPRECLVLCYAFFGVAILVNIVKDSLHSNWGRFIPLPMAMAIPFFLGPYFAIDMCVGSLILFIWERVDAAKAEAFGTAVASGLICGDGIWSLPSSVLAIAGVNPPVCMKFLSSATNSKVDNFLKGSI

SEQ ID NO: 13 - DNA, 2327 bp, Arabidopsis sp.
  CDS (42)..(2075)
ccgccggtgaaaagaaactcattcattgaattagctaaaacatggggacggagatccctagatcggcggagatatcagaggcattgttaccaccggaatctgagaaaaccgtgacggcgacggaggagcatgtacctgagtggaaagagcagataacgattcgaggattaaccgtgagtgcgttgctcgggactttgttctgtatcattactcataaactaaatctgacggttggaatcatcccgtcgttgaacgtcgctgcgggattactcggtttcttcttcgttaagtcgtggactggtttcttatcgaagctagggtttacagtgaagcctttcactaagcaagagaacaccgttattcagacttgtgttgtcgctgtcgcttgctatggcctcgcctttagcggaggatttggttcatatttgattgctatggatgagaagacatataagctcattggtgctgattatcctggaaaccatgcagaagatgttataaatccaggattgtggtggatgattgggtttttatttgtagtcagcttcttgggactctttagtctcgttccgttacgcaaggtgatggttttggactacaaacttacttatcctagtggaaccgccacagcaatgttgattaatagcttccacaccaacactggagctgagcttgcagggaaccaggttaaatgtcttggaaaatacctgagccttagcttaatttggagttgtttcaaatggttcttcagtggtattggagatgcatgggaattgaaattaaccacccttggtttgacgttattcaagaacacgttttactttgacttcagtcctacttatattggatgcggccttatatgcccccatatagtgaactgctcggttcttctcggtgcgatcatttcttgggggattctatggccatttgtatcacagcatgctggggattggtatcctgctgaccttgggtccaacgatttcaaaggtctctacgggtataaggtctttatcgccattgccattatccttggtgatggtctttacaatcttgtcaagatcatcgctgtcactgtgaaggaattatgcagcagcaggtctagacgactcaatctacccattgttaccgacggtgtagatgacagtgaagcttctgagatactgctggtgaagaagaaaagagatgaagtgtttctgaaggaccgtataccccttgaatttgcgattgctggttatgtgggtcttgcagctatctcaactgccacaatcccaataatatttccaccactgaaatggtactttgtcctgtgttcttatttcattgcacccgctctcgccttctgcaactcttatggaacggggctcacagactggagcctagcatcaacctatggaaagatcggtcttttcataatcgcttccgttgtagaaagtgatggtggtgtcattgcgggtttagctgcctgtggtgttatgatgtcaatcgtctccaccgcagctgatctcatgcaagacttcaaaacaggttacctcactttatcatctgcaaagtccatgtttgtaagccagctcgtgggaaccgcaatgggctgtgtaatcgctccactcacattctggctgttctggactgcctttgacattggagatcccaatggtccgtacaaagcaccttacgcagtgattttccgtgaaatggcgattctcggaattgagggctttgctgaattgcctaagcactgtttggctctttgttacgggtttttcatagcagctttgattgtgaatctcttaagagatattacaccgcctaagatctctcagttcatcccgatcccaatggcaatggctgtcccattctacattggagcttacttcgccattgacatgtttgttgggactgtgatattgttcgtatgggaacggatcaacaggaaagatgcagaggactttgcaggtgcagtagcatcaggactgatctgtggtgatgggatctggactataccatccgcaatcctttcaatcttaaggatcaaccctcccatttgtatgtactttggaccgtcctcagcaagatagagatgccactgtggtcataatcgcaaccgcaactgtgagtcatacaaaaggaaacttatccgagagtcttgtggccgtcagaatcgaaacagttgtcttcatatgttgtgtagatatgtttagtatctcagctcgcaagaacaaaatgttgttgcaaatctcttcaaatgttttagtcaatgttgttgtatgtccccgggatgttgtctccttgaatgctttcattcacaagctcaaagagttttacgaaca

SEQ ID NO: 14 - protein, 677 aa, Arabidopsis sp.
MGTEIPRSAEISEALLPPESEKTVTATEEHVPEWKEQITIRGLTVSALLGTLFCIITHKLNLTVGIIPSLNVAAGLLGFFFVKSWTGFLSKLGFTVKPFTKQENTVIQTCVVAVACYGLAFSGGFGSYLIAMDEKTYKLIGADYPGNHAEDVINPGLWWMIGFLFVVSFLGLFSLVPLRKVMVLDYKLTYPSGTATAMLINSFHTNTGAELAGNQVKCLGKYLSLSLIWSCFKWFFSGIGDAWELKLTTLGLTLFKNTFYFDFSPTYIGCGLICPHIVNCSVLLGAIISWGILWPFVSQHAGDWYPADLGSNDFKGLYGYKVFIAIAIILGDGLYNLVKIIAVTVKELCSSRSRRLNLPIVTDGVDDSEASEILLVKKKRDEVFLKDRIPLEFAIAGYVGLAAISTATIPIIFPPLKWYFVLCSYFIAPALAFCNSYGTGLTDWSLASTYGKIGLFIIASVVESDGGVIAGLAACGVMMSIVSTAADLMQDFKTGYLTLSSAKSMFVSQLVGTAMGCVIAPLTFWLFWTAFDIGDPNGPYKAPYAVIFREMAILGIEGFAELPKHCLALCYGFFIAALIVNLLRDITPPKISQFIPIPMAMAVPFYIGAYFAIDMFVGTVILFVWERINRKDAEDFAGAVASGLICGDGIWTIPSAILSILRINPPICMYFGPSSAR

SEQ ID NO: 15 - DNA, 2344 bp, Arabidopsis sp.
  CDS (112)..(2178)
gtcctcttctctcgccgatctcttctcttcagatcctctcctctataaaatctctccctctctcgttcccctcctcagatctgactcaaatcaaatcaaaaactctcagctatggaagtcgaacgatcgaagaaagacgacgatctaaacaatggatctaaatccaacgaagaagaagaaatctccgtggagagaatcttcgaagagagtaacgaaatcccaccgccgtggcaaaaacagctcacgttcagagctctaatcgtgagtttcatactcgcgattctctttacctttgtggtgatgaaactgaatctaacgactgggattattccgtcgctgaatatctccgccggtttgcttggtttcttctttgttaaaagttggactaagattcttaataaagctggttttcttaaacaaccgtttacacggcaagagaatactgtgattcagacttgtgtcgtcgcctcctccggcatcgcctttagcggtgggtttggtagttatctgtttggaatgagtgatgttgttgctaaacaatctgcggaagccaatactccgttgaacataaagaatccacatttgggttggatgataggatttctctttgtcgttagctttcttggtcttttctccgtcgttcctcttcgaaagatcatgatcgtggacttcaaattgacatatcctagtggtactgccactgcccatctcattaacagcttccacacacctcaaggtgccaaactcgcaaagaagcaagtcagagcattgggcaagttcttctcattcagtttcttatggggtttctttcaatggttctttgctactggtgacggttgtggtttcgccaacttccctacatttggtctcaaagcctatgaaaacaaattctactttgatttctctgctacatatgttggtgttggaatgatttgtccatatcttatcaatgtgtctcttctcatcggagcaattctctcatggggtgttatgtggcctctcattggtgcccaaaaaggcaaatggtatgctgctgatctctcatcaaccagtctccatggtcttcaaggctacagggtgtttatagccatagctatgatcctcggtgatggtctctacaacttcatcaaagtcttaggccgcactgtcttcggtctatacaagcaattcaagaacaaagatgttcttcctatcaacgaccatacatcaacagcccctgtaaccatttcctacgacgacaaaagaagaacagagcttttcctcaaagacagaatcccatcatggtttgctgtaaccggttacgtagtattggctatagtctcaatcatcacagttccacatatcttccatcagctaaaatggtaccacattttgatcatgtacataatcgcccctgtcttagccttttgcaacgcctacggttgcggactcactgactggtccttagcttccacttatggaaaactcgccattttcaccatcggagcctgggctggtgcatctaacggaggcgtcttagctggacttgcagcttgtggtgtcatgatgaacattgtctcaacagcttctgatcttatgcaagatttcaaaaccggttacatgacattagcctcaccaagatcaatgttcttgagccaagccattggaaccgccatgggatgtgtgatctctccttgcgtcttctggctattctacaaggcgttccctgactttggccaacccgggaccgcctatcccgctccatacgccttagtctacagaaacatgtctatactcggagtcgaaggattctctgctttgcccaaacattgcctcatgctctgctacatattcttcgcagcagcagtgatcgtaaacggtataagggatgcccttgggccaaagtgggctcggtttatcccgctcccaatggctatggctatacccttctaccttggaggttacttcacaatagatatgtgtctgggaagtctcatactgttcatctggaggaagctgaataaaccaaaggccgatgcgtactcttctgctgttgcttctggtttgatctgtggtgaaggtatttggacgttaccaagctctatccttgccttggctggtgtcaaggctcctatttgcatgaagttcttgtccatggcttccaacaacaaggttgatgccttcttaaacccttcttaacattcttttttttttttttagagtttggtttttaattttctacgtttctttgtctagttttaattggctttttaggtcctcatgtgtctgtctttaagttggtggtttcttttctttctactcaaatatgtaaaaaaagggaatgaatgaattatttggttttctg

SEQ ID NO: 16 - protein, 688 aa, Arabidopsis sp.
MEVERSKKDDDLNNGSKSNEEEEISVERIFEESNEIPPPWQKQLTFRALIVSFILAILFTFVVMKLNLTTGIIPSLNISAGLLGFFFVKSWTKILNKAGFLKQPFTRQENTVIQTCVVASSGIAFSGGFGSYLFGMSDVVAKQSAEANTPLNIKNPHLGWMIGFLFVVSFLGLFSVVPLRKIMIVDFKLTYPSGTATAHLINSFHTPQGAKLAKKQVRALGKFFSFSFLWGFFQWFFATGDGCGFANFPTFGLKAYENKFYFDFSATYVGVGMICPYLINVSLLIGAILSWGVMWPLIGAQKGKWYAADLSSTSLHGLQGYRVFIAIAMILGDGLYNFIKVLGRTVFGLYKQFKNKDVLPINDHTSTAPVTISYDDKRRTELFLKDRIPSWFAVTGYVVLAIVSIITVPHIFHQLKWYHILIMYIIAPVLAFCNAYGCGLTDWSLASTYGKLAIFTIGAWAGASNGGVLAGLAACGVMMNIVSTASDLMQDFKTGYMTLASPRSMFLSQAIGTAMGCVISPCVFWLFYKAFPDFGQPGTAYPAPYALVYRNMSILGVEGFSALPKHCLMLCYIFFAAAVIVNGIRDALGPKWARFIPLPMAMAIPFYLGGYFTIDMCLGSLILFIWRKLNKPKADAYSSAVASGLICGEGIWTLPSSILALAGVKAPICMKFLSMASNNKVDAFLNPS

SEQ ID NO: 17 - DNA, 2311 bp, Arabidopsis sp.
  CDS (49)..(2223)
tcttgttcaccatctctcttatttcgcagatctaaagagaaaaaaaccatgagaaaaggaggtttaactcctgacagagatcgtcagattgaggaacatgagttgcaagaaactgggataagtcctgacatagagaggttaaagaggaacattaatgctacaccataccaaagagaagaagaggaagaagatagagaggaacaggaagaatccgtggagggaatattcgaaagcagagaggttccttcgtggaagaagcagttaacaattagggcttttgtggttagctttgcgctaagcattttgtttagcttcgttgtgatgaagcttaacctcacaacaggaatcattccttcgcttaatgtatctgctggtcttttggggttcttcttcgtcgagacatggactaagatgcttcataaatctggtcttttgaaacagccctttacaagacaagagaacaccgttattcagacttgtgtagttgcttcctcaggaattgcctttagcggaggatttgggacttacctatttgcaatgagccatcgaatagcagaccaatcaggagacgttgcccgtggcgttaaggatccttcattgggttggatgatagcttttctctttgttgtcagctttcttggcctcttctctgttgttcctcttagaaagataatgataatagacttcaaactaccatacccaagtggcactgcaactgctcatcttatcaacagctttcatactcctcaaggggcaaagctagccaagaaacaagtccgagtcttggggaaattcttctccttcagcttcttttggggtttcttccaatggttctttactgcaggtgaaaattgtgggtttaacagcttccctacttttggactcagagcttaccaatataagttctactttgatttttctgcaacatatgtgggtgttggtatgatatgcccatacataatcaacatctctcttctattgggtggaattctctcttggggactaatgtggcctctcattgaaaccagaaaaggagattggttcccttctaatgttgactctagcagcatgaacggtcttcaggcttacaaggtgttcatagctgtcgctacgatcctaggagatggtctatacaacttttgcaaggtgttgatccgaaccttttcaggactgatctcgcagatccgaggtaaagctggttcgagaagctctcttgcacacaaagaagaccctcctgcttcccctgcttccccattgaccccaaggatatcttatgatgaccaacgacgaacaagattcttcctcaaggatcaaataccgtcttggtttgctgttggaggctatgtggttatatctgcagtgtctacagctatacttcctcacatgttctctcagctgagatggtactacattatagtcatctatatctttgctcctatcttagctttctgcaatgcttacggagccgggctcactgattggtcgctagcatcaacctatggaaagcttgccatatttacaattggagtatgggctggttcggatcatggaggtcttctggcaggcttagcggcttgtggagtcatgatgaatatagtatcgacagcttcagacctgacgcaggatttcaaaactggttacctaacattgtcatctccaagagccatgtttgtgagccaggtaattggaacagctatgggctgcttggtttcaccctgcgtattctggcttttctacaaggcgtttgatgatcttggtctaccaaacagtgaataccctgctccgtttgctactgtgtaccgaagtatggctaaactcggggttgaaggcgtctcgtctctaccaagagactgtcttatgctgtgctacgtgtttttcggtgtggccatactcattaacttaataaaagattgtcttggtaacaggtggggaaggtttgttcctcttcccatggcaatggctataccgtttttcttggggccttactttgcaattgacatgtgcgtggggagttttattttgtttgtctgggagaggttagatgcgccaaaggctgaagcatttgcaacagcagtggcttctggtttgatatgtggagatggaatctggactttgccaagttcagtgcttgctatagctggagttaaacctcctatttgcatgaagtttctttcagctgcaactaatcacagagtcgacaagttcctgcaaggatcctcttagttttttttttaagctaaatgagatggatctgttgagaagtaaagtgttagtttttttaaagctcaatgagatggatctgttgaagaag

SEQ ID NO: 18 - protein, 724 aa, Arabidopsis sp.
MRKGGLTPDRDRQIEEHELQETGISPDIERLKRNINATPYQREEEEEDREEQEESVEGIFESREVPSWKKQLTIRAFVVSFALSILFSFVVMKLNLTTGIIPSLNVSAGLLGFFFVETWTKMLHKSGLLKQPFTRQENTVIQTCVVASSGIAFSGGFGTYLFAMSHRIADQSGDVARGVKDPSLGWMIAFLFVVSFLGLFSVVPLRKIMIIDFKLPYPSGTATAHLINSFHTPQGAKLAKKQVRVLGKFFSFSFFWGFFQWFFTAGENCGFNSFPTFGLRAYQYKFYFDFSATYVGVGMICPYIINISLLLGGILSWGLMWPLIETRKGDWFPSNVDSSSMNGLQAYKVFIAVATILGDGLYNFCKVLIRTFSGLISQIRGKAGSRSSLAHKEDPPASPASPLTPRISYDDQRRTRFFLKDQIPSWFAVGGYVVISAVSTAILPHMFSQLRWYYIIVIYIFAPILAFCNAYGAGLTDWSLASTYGKLAIFTIGVWAGSDHGGLLAGLAACGVMMNIVSTASDLTQDFKTGYLTLSSPRAMFVSQVIGTAMGCLVSPCVFWLFYKAFDDLGLPNSEYPAPFATVYRSMAKLGVEGVSSLPRDCLMLCYVFFGVAILINLIKDCLGNRWGRFVPLPMAMAIPFFLGPYFAIDMCVGSFILFVWERLDAPKAEAFATAVASGLICGDGIWTLPSSVLAIAGVKPPICMKFLSAATNHRVDKFLQGSS

SEQ ID NO: 19 - protein, 11 aa, artificial
  motif similar to region near amino terminus of yellow stripe1 gene
REKELELELER

SEQ ID NO: 20 - protein, 5 aa, Saccharomyces cerevisiae
  Protein motif involved in iron III transport
REGLE